SEQUENCE LISTING (340 sequences)

SEQ ID NO: 1 - DNA, 44 bp, Artificial Sequence
  Description of Artificial Sequence Synthetic oligonucleotide
gcatggccaattgcataaccggttggccaattgcatggccaatt

SEQ ID NO: 2 - DNA, 16 bp, Artificial Sequence
  Description of Artificial Sequence Synthetic oligonucleotide
gcatggccaattgcat

SEQ ID NO: 3 - DNA, 28 bp, Artificial Sequence
  Description of Artificial Sequence Synthetic oligonucleotide
aaccggttggccaattgcatggccaatt

SEQ ID NO: 4 - DNA, 14 bp, Artificial Sequence
  Description of Artificial Sequence Synthetic oligonucleotide
gcatggccaattgc

SEQ ID NO: 5 - DNA, 23 bp, Artificial Sequence
  Description of Artificial Sequence Synthetic oligonucleotide
gttggccaattgcatggccaatt

SEQ ID NO: 6 - protein, 15 aa, Artificial Sequence
  Description of Artificial Sequence Synthetic peptide
RMWPSSTVNLSAGRR

SEQ ID NO: 7 - DNA, 10 bp, Artificial Sequence
  Description of Artificial Sequence Synthetic oligonucleotide
nnnnngatcc

SEQ ID NO: 8 - DNA, 10 bp, Artificial Sequence
  Description of Artificial Sequence Synthetic oligonucleotide
gaannnnttc

SEQ ID NO: 9 - DNA, 11 bp, Artificial Sequence
  Description of Artificial Sequence Synthetic oligonucleotide
gacnnnnngtc

SEQ ID NO: 10 - DNA, 12 bp, Artificial Sequence
  Description of Artificial Sequence Synthetic oligonucleotide
nnnnnngtcttc

SEQ ID NO: 11 - DNA, 13 bp, Artificial Sequence
  Description of Artificial Sequence Synthetic oligonucleotide
gcagcnnnnnnnn

SEQ ID NO: 12 - DNA, 17 bp, Artificial Sequence
  Description of Artificial Sequence Synthetic oligonucleotide
nnnnnnnnnnnngctgc

SEQ ID NO: 13 - DNA, 34 bp, Artificial Sequence
  Description of Artificial Sequence Synthetic oligonucleotide
nnnnnnnnnncgannnnnntgcnnnnnnnnnnnn

SEQ ID NO: 14 - DNA, 34 bp, Artificial Sequence
  Description of Artificial Sequence Synthetic oligonucleotide
nnnnnnnnnngcannnnnntcgnnnnnnnnnnnn

SEQ ID NO: 15 - DNA, 11 bp, Artificial Sequence
  Description of Artificial Sequence Synthetic oligonucleotide
gccnnnnnggc

SEQ ID NO: 16 - DNA, 22 bp, Artificial Sequence
  Description of Artificial Sequence Synthetic oligonucleotide
ctggagnnnnnnnnnnnnnnnn

SEQ ID NO: 17 - DNA, 20 bp, Artificial Sequence
  Description of Artificial Sequence Synthetic oligonucleotide
nnnnnnnnnnnnnnctccag

SEQ ID NO: 18 - DNA, 12 bp, Artificial Sequence
  Description of Artificial Sequence Synthetic oligonucleotide
nnnnnngtcttc

SEQ ID NO: 19 - DNA, 11 bp, Artificial Sequence
  Description of Artificial Sequence Synthetic oligonucleotide
nnnnngagacc

SEQ ID NO: 20 - DNA, 10 bp, Artificial Sequence
  Description of Artificial Sequence Synthetic oligonucleotide
gatnnnnatc

SEQ ID NO: 21 - DNA, 22 bp, Artificial Sequence
  Description of Artificial Sequence Synthetic oligonucleotide
gtgcagnnnnnnnnnnnnnnnn

SEQ ID NO: 22 - DNA, 20 bp, Artificial Sequence
  Description of Artificial Sequence Synthetic oligonucleotide
nnnnnnnnnnnnnnctgcac

SEQ ID NO: 23 - DNA, 11 bp, Artificial Sequence
  Description of Artificial Sequence Synthetic oligonucleotide
ccnnnnnnngg

SEQ ID NO: 24 - DNA, 10 bp, Artificial Sequence
  Description of Artificial Sequence Synthetic oligonucleotide
nnnnngagac

SEQ ID NO: 25 - DNA, 10 bp, Artificial Sequence
  Description of Artificial Sequence Synthetic oligonucleotide
acctgcnnnn

SEQ ID NO: 26 - DNA, 14 bp, Artificial Sequence
  Description of Artificial Sequence Synthetic oligonucleotide
nnnnnnnngcaggt

SEQ ID NO: 27 - DNA, 12 bp, Artificial Sequence
  Description of Artificial Sequence Synthetic oligonucleotide
ccannnnnntgg

SEQ ID NO: 28 - DNA, 12 bp, Artificial Sequence
  Description of Artificial Sequence Synthetic oligonucleotide
gacnnnnnngtc

SEQ ID NO: 29 - DNA, 10 bp, Artificial Sequence
  Description of Artificial Sequence Synthetic oligonucleotide
nnnngaagag

SEQ ID NO: 30 - DNA, 22 bp, Artificial Sequence
  Description of Artificial Sequence Synthetic oligonucleotide
ctgaagnnnnnnnnnnnnnnnn

SEQ ID NO: 31 - DNA, 20 bp, Artificial Sequence
  Description of Artificial Sequence Synthetic oligonucleotide
nnnnnnnnnnnnnncttcag

SEQ ID NO: 32 - DNA, 11 bp, Artificial Sequence
  Description of Artificial Sequence Synthetic oligonucleotide
cctnnnnnagg

SEQ ID NO: 33 - DNA, 11 bp, Artificial Sequence
  Description of Artificial Sequence Synthetic oligonucleotide
nnnnngagacg

SEQ ID NO: 34 - DNA, 14 bp, Artificial Sequence
  Description of Artificial Sequence Synthetic oligonucleotide
ggatgnnnnnnnnn

SEQ ID NO: 35 - DNA, 18 bp, Artificial Sequence
  Description of Artificial Sequence Synthetic oligonucleotide
nnnnnnnnnnnnncatcc

SEQ ID NO: 36 - DNA, 10 bp, Artificial Sequence
  Description of Artificial Sequence Synthetic oligonucleotide
gacgcnnnnn

SEQ ID NO: 37 - DNA, 15 bp, Artificial Sequence
  Description of Artificial Sequence Synthetic oligonucleotide
nnnnnnnnnngcgtc

SEQ ID NO: 38 - DNA, 13 bp, Artificial Sequence
  Description of Artificial Sequence Synthetic oligonucleotide
ggtgannnnnnnn

SEQ ID NO: 39 - DNA, 12 bp, Artificial Sequence
  Description of Artificial Sequence Synthetic oligonucleotide
nnnnnnntcacc

SEQ ID NO: 40 - DNA, 13 bp, Artificial Sequence
  Description of Artificial Sequence Synthetic oligonucleotide
gaagannnnnnnn

SEQ ID NO: 41 - DNA, 12 bp, Artificial Sequence
  Description of Artificial Sequence Synthetic oligonucleotide
nnnnnnntcttc

SEQ ID NO: 42 - DNA, 11 bp, Artificial Sequence
  Description of Artificial Sequence Synthetic oligonucleotide
cctcnnnnnnn

SEQ ID NO: 43 - DNA, 10 bp, Artificial Sequence
  Description of Artificial Sequence Synthetic oligonucleotide
nnnnnngagg

SEQ ID NO: 44 - DNA, 11 bp, Artificial Sequence
  Description of Artificial Sequence Synthetic oligonucleotide
ccannnnntgg

SEQ ID NO: 45 - DNA, 9 bp, Artificial Sequence
  Description of Artificial Sequence Synthetic oligonucleotide
gagtcnnnn

SEQ ID NO: 46 - DNA, 10 bp, Artificial Sequence
  Description of Artificial Sequence Synthetic oligonucleotide
nnnnngactc

SEQ ID NO: 47 - DNA, 10 bp, Artificial Sequence
  Description of Artificial Sequence Synthetic oligonucleotide
gcatcnnnnn

SEQ ID NO: 48 - DNA, 14 bp, Artificial Sequence
  Description of Artificial Sequence Synthetic oligonucleotide
nnnnnnnnngatgc

SEQ ID NO: 49 - DNA, 13 bp, Artificial Sequence
  Description of Artificial Sequence Synthetic oligonucleotide
ggccnnnnnggcc

SEQ ID NO: 50 - DNA, 15 bp, Artificial Sequence
  Description of Artificial Sequence Synthetic oligonucleotide
ccannnnnnnnntgg

SEQ ID NO: 51 - DNA, 6 bp, Artificial Sequence
  Description of Artificial Sequence Synthetic oligonucleotide
acngan

SEQ ID NO: 52 - DNA, 10 bp, Artificial Sequence
  Description of Artificial Sequence Synthetic oligonucleotide
nnnnntcngt

SEQ ID NO: 53 - DNA, 6 bp, Artificial Sequence
  Description of Artificial Sequence Synthetic oligonucleotide
raatty

SEQ ID NO: 54 - DNA, 6 bp, Artificial Sequence
  Description of Artificial Sequence Synthetic oligonucleotide
aagctt

SEQ ID NO: 55 - DNA, 6 bp, Artificial Sequence
  Description of Artificial Sequence Synthetic oligonucleotide
aacgtt

SEQ ID NO: 56 - DNA, 6 bp, Artificial Sequence
  Description of Artificial Sequence Synthetic oligonucleotide
aatatt

SEQ ID NO: 57 - DNA, 6 bp, Artificial Sequence
  Description of Artificial Sequence Synthetic oligonucleotide
acrygt

SEQ ID NO: 58 - DNA, 6 bp, Artificial Sequence
  Description of Artificial Sequence Synthetic oligonucleotide
accggt

SEQ ID NO: 59 - DNA, 6 bp, Artificial Sequence
  Description of Artificial Sequence Synthetic oligonucleotide
acatgt

SEQ ID NO: 60 - DNA, 10 bp, Artificial Sequence
  Description of Artificial Sequence Synthetic oligonucleotide
acctgcnnnn

SEQ ID NO: 61 - DNA, 14 bp, Artificial Sequence
  Description of Artificial Sequence Synthetic oligonucleotide
nnnnnnnngcaggt

SEQ ID NO: 62 - DNA, 6 bp, Artificial Sequence
  Description of Artificial Sequence Synthetic oligonucleotide
actggn

SEQ ID NO: 63 - DNA, 6 bp, Artificial Sequence
  Description of Artificial Sequence Synthetic oligonucleotide
nccagt

SEQ ID NO: 64 - DNA, 6 bp, Artificial Sequence
  Description of Artificial Sequence Synthetic oligonucleotide
rccggy

SEQ ID NO: 65 - DNA, 4 bp, Artificial Sequence
  Description of Artificial Sequence Synthetic oligonucleotide
acgt

SEQ ID NO: 66 - DNA, 6 bp, Artificial Sequence
  Description of Artificial Sequence Synthetic oligonucleotide
acgcgt

SEQ ID NO: 67 - DNA, 6 bp, Artificial Sequence
  Description of Artificial Sequence Synthetic oligonucleotide
rcatgy

SEQ ID NO: 68 - DNA, 7 bp, Artificial Sequence
  Description of Artificial Sequence Synthetic oligonucleotide
accwggt

SEQ ID NO: 69 - DNA, 6 bp, Artificial Sequence
  Description of Artificial Sequence Synthetic oligonucleotide
actagt

SEQ ID NO: 70 - DNA, 6 bp, Artificial Sequence
  Description of Artificial Sequence Synthetic oligonucleotide
aggcct

SEQ ID NO: 71 - DNA, 4 bp, Artificial Sequence
  Description of Artificial Sequence Synthetic oligonucleotide
agct

SEQ ID NO: 72 - DNA, 6 bp, Artificial Sequence
  Description of Artificial Sequence Synthetic oligonucleotide
agatct

SEQ ID NO: 73 - DNA, 6 bp, Artificial Sequence
  Description of Artificial Sequence Synthetic oligonucleotide
rgatcy

SEQ ID NO: 74 - DNA, 7 bp, Artificial Sequence
  Description of Artificial Sequence Synthetic oligonucleotide
rggnccy

SEQ ID NO: 75 - DNA, 6 bp, Artificial Sequence
  Description of Artificial Sequence Synthetic oligonucleotide
agcgct

SEQ ID NO: 76 - DNA, 6 bp, Artificial Sequence
  Description of Artificial Sequence Synthetic oligonucleotide
rgcgcy

SEQ ID NO: 77 - DNA, 7 bp, Artificial Sequence
  Description of Artificial Sequence Synthetic oligonucleotide
rggwccy

SEQ ID NO: 78 - DNA, 6 bp, Artificial Sequence
  Description of Artificial Sequence Synthetic oligonucleotide
agtact

SEQ ID NO: 79 - DNA, 6 bp, Artificial Sequence
  Description of Artificial Sequence Synthetic oligonucleotide
attaat

SEQ ID NO: 80 - DNA, 6 bp, Artificial Sequence
  Description of Artificial Sequence Synthetic oligonucleotide
atcgat

SEQ ID NO: 81 - DNA, 6 bp, Artificial Sequence
  Description of Artificial Sequence Synthetic oligonucleotide
atgcat

SEQ ID NO: 82 - DNA, 8 bp, Artificial Sequence
  Description of Artificial Sequence Synthetic oligonucleotide
atttaaat

SEQ ID NO: 83 - DNA, 9 bp, Artificial Sequence
  Description of Artificial Sequence Synthetic oligonucleotide
cagnnnctg

SEQ ID NO: 84 - DNA, 6 bp, Artificial Sequence
  Description of Artificial Sequence Synthetic oligonucleotide
cacgtg

SEQ ID NO: 85 - DNA, 9 bp, Artificial Sequence
  Description of Artificial Sequence Synthetic oligonucleotide
cacnnngtg

SEQ ID NO: 86 - DNA, 6 bp, Artificial Sequence
  Description of Artificial Sequence Synthetic oligonucleotide
yacgtr

SEQ ID NO: 87 - DNA, 6 bp, Artificial Sequence
  Description of Artificial Sequence Synthetic oligonucleotide
caattg

SEQ ID NO: 88 - DNA, 6 bp, Artificial Sequence
  Description of Artificial Sequence Synthetic oligonucleotide
catatg

SEQ ID NO: 89 - DNA, 4 bp, Artificial Sequence
  Description of Artificial Sequence Synthetic oligonucleotide
catg

SEQ ID NO: 90 - DNA, 6 bp, Artificial Sequence
  Description of Artificial Sequence Synthetic oligonucleotide
cmgckg

SEQ ID NO: 91 - DNA, 6 bp, Artificial Sequence
  Description of Artificial Sequence Synthetic oligonucleotide
cagctg

SEQ ID NO: 92 - DNA, 8 bp, Artificial Sequence
  Description of Artificial Sequence Synthetic oligonucleotide
crccggyg

SEQ ID NO: 93 - DNA, 18 bp, Artificial Sequence
  Description of Artificial Sequence Synthetic oligonucleotide
nnnnnnnnnnnnncatcc

SEQ ID NO: 94 - DNA, 14 bp, Artificial Sequence
  Description of Artificial Sequence Synthetic oligonucleotide
ggatgnnnnnnnnn

SEQ ID NO: 95 - DNA, 4 bp, Artificial Sequence
  Description of Artificial Sequence Synthetic oligonucleotide
ccgc

SEQ ID NO: 96 - DNA, 7 bp, Artificial Sequence
  Description of Artificial Sequence Synthetic oligonucleotide
cctnagg

SEQ ID NO: 97 - DNA, 5 bp, Artificial Sequence
  Description of Artificial Sequence Synthetic oligonucleotide
ccwgg

SEQ ID NO: 98 - DNA, 6 bp, Artificial Sequence
  Description of Artificial Sequence Synthetic oligonucleotide
cctagg

SEQ ID NO: 99 - DNA, 6 bp, Artificial Sequence
  Description of Artificial Sequence Synthetic oligonucleotide
ccnngg

SEQ ID NO: 100 - DNA, 5 bp, Artificial Sequence
  Description of Artificial Sequence Synthetic oligonucleotide
ccwgg

SEQ ID NO: 101 - DNA, 6 bp, Artificial Sequence
  Description of Artificial Sequence Synthetic oligonucleotide
ccrygg

SEQ ID NO: 102 - DNA, 6 bp, Artificial Sequence
  Description of Artificial Sequence Synthetic oligonucleotide
cycgrg

SEQ ID NO: 103 - DNA, 4 bp, Artificial Sequence
  Description of Artificial Sequence Synthetic oligonucleotide
ccgg

SEQ ID NO: 104 - DNA, 6 bp, Artificial Sequence
  Description of Artificial Sequence Synthetic oligonucleotide
ccgcgg

SEQ ID NO: 105 - DNA, 11 bp, Artificial Sequence
  Description of Artificial Sequence Synthetic oligonucleotide
cctcnnnnnnn

SEQ ID NO: 106 - DNA, 10 bp, Artificial Sequence
  Description of Artificial Sequence Synthetic oligonucleotide
nnnnnngagg

SEQ ID NO: 107 - DNA, 5 bp, Artificial Sequence
  Description of Artificial Sequence Synthetic oligonucleotide
ccsgg

SEQ ID NO: 108 - DNA, 6 bp, Artificial Sequence
  Description of Artificial Sequence Synthetic oligonucleotide
ccatgg

SEQ ID NO: 109 - DNA, 5 bp, Artificial Sequence
  Description of Artificial Sequence Synthetic oligonucleotide
ccngg

SEQ ID NO: 110 - DNA, 6 bp, Artificial Sequence
  Description of Artificial Sequence Synthetic oligonucleotide
cccggg

SEQ ID NO: 111 - DNA, 8 bp, Artificial Sequence
  Description of Artificial Sequence Synthetic oligonucleotide
cctgcagg

SEQ ID NO: 112 - DNA, 6 bp, Artificial Sequence
  Description of Artificial Sequence Synthetic oligonucleotide
ccwwgg

SEQ ID NO: 113 - DNA, 6 bp, Artificial Sequence
  Description of Artificial Sequence Synthetic oligonucleotide
cccggg

SEQ ID NO: 114 - DNA, 6 bp, Artificial Sequence
  Description of Artificial Sequence Synthetic oligonucleotide
nccagt

SEQ ID NO: 115 - DNA, 6 bp, Artificial Sequence
  Description of Artificial Sequence Synthetic oligonucleotide
actggn

SEQ ID NO: 116 - DNA, 34 bp, Artificial Sequence; identical to SEQ ID NO: 13
  Description of Artificial Sequence Synthetic oligonucleotide

SEQ ID NO: 117 - DNA, 34 bp, Artificial Sequence; identical to SEQ ID NO: 14
  Description of Artificial Sequence Synthetic oligonucleotide

SEQ ID NO: 118 - DNA, 6 bp, Artificial Sequence
  Description of Artificial Sequence Synthetic oligonucleotide
cgrycg

SEQ ID NO: 119 - DNA, 6 bp, Artificial Sequence
  Description of Artificial Sequence Synthetic oligonucleotide
cgtacg

SEQ ID NO: 120 - DNA, 4 bp, Artificial Sequence
  Description of Artificial Sequence Synthetic oligonucleotide
cgcg

SEQ ID NO: 121 - DNA, 6 bp, Artificial Sequence
  Description of Artificial Sequence Synthetic oligonucleotide
cggccg

SEQ ID NO: 122 - DNA, 6 bp, Artificial Sequence
  Description of Artificial Sequence Synthetic oligonucleotide
yggccr

SEQ ID NO: 123 - DNA, 7 bp, Artificial Sequence
  Description of Artificial Sequence Synthetic oligonucleotide
cgtctcn

SEQ ID NO: 124 - DNA, 11 bp, Artificial Sequence
  Description of Artificial Sequence Synthetic oligonucleotide
nnnnngagacg

SEQ ID NO: 125 - DNA, 6 bp, Artificial Sequence
  Description of Artificial Sequence Synthetic oligonucleotide
cgatcg

SEQ ID NO: 126 - DNA, 7 bp, Artificial Sequence
  Description of Artificial Sequence Synthetic oligonucleotide
cggwccg

SEQ ID NO: 127 - DNA, 6 bp, Artificial Sequence
  Description of Artificial Sequence Synthetic oligonucleotide
cttaag

SEQ ID NO: 128 - DNA, 4 bp, Artificial Sequence
  Description of Artificial Sequence Synthetic oligonucleotide
ctag

SEQ ID NO: 129 - DNA, 22 bp, Artificial Sequence
  Description of Artificial Sequence Synthetic oligonucleotide
ctggagnnnnnnnnnnnnnnnn

SEQ ID NO: 130 - DNA, 20 bp, Artificial Sequence
  Description of Artificial Sequence Synthetic oligonucleotide
nnnnnnnnnnnnnnctccag

SEQ ID NO: 131 - DNA, 5 bp, Artificial Sequence
  Description of Artificial Sequence Synthetic oligonucleotide
ctnag

SEQ ID NO: 132 - DNA, 7 bp, Artificial Sequence
  Description of Artificial Sequence Synthetic oligonucleotide
ctcttcn

SEQ ID NO: 133 - DNA, 10 bp, Artificial Sequence
  Description of Artificial Sequence Synthetic oligonucleotide
nnnngaagag

SEQ ID NO: 134 - DNA, 22 bp, Artificial Sequence
  Description of Artificial Sequence Synthetic oligonucleotide
ctgaagnnnnnnnnnnnnnnnn

SEQ ID NO: 135 - DNA, 20 bp, Artificial Sequence
  Description of Artificial Sequence Synthetic oligonucleotide
nnnnnnnnnnnnnncttcag

SEQ ID NO: 136 - DNA, 6 bp, Artificial Sequence
  Description of Artificial Sequence Synthetic oligonucleotide
ctcgag

SEQ ID NO: 137 - DNA, 6 bp, Artificial Sequence
  Description of Artificial Sequence Synthetic oligonucleotide
ctgcag

SEQ ID NO: 138 - DNA, 6 bp, Artificial Sequence
  Description of Artificial Sequence Synthetic oligonucleotide
ctryag

SEQ ID NO: 139 - DNA, 20 bp, Artificial Sequence
  Description of Artificial Sequence Synthetic oligonucleotide
nnnnnnnnnnnnnnctccag

SEQ ID NO: 140 - DNA, 22 bp, Artificial Sequence
  Description of Artificial Sequence Synthetic oligonucleotide
ctggagnnnnnnnnnnnnnnnn

SEQ ID NO: 141 - DNA, 20 bp, Artificial Sequence
  Description of Artificial Sequence Synthetic oligonucleotide
nnnnnnnnnnnnnnctgcac

SEQ ID NO: 142 - DNA, 22 bp, Artificial Sequence
  Description of Artificial Sequence Synthetic oligonucleotide
gtgcagnnnnnnnnnnnnnnnn

SEQ ID NO: 143 - DNA, 20 bp, Artificial Sequence
  Description of Artificial Sequence Synthetic oligonucleotide
nnnnnnnnnnnnnncttcag

SEQ ID NO: 144 - DNA, 22 bp, Artificial Sequence
  Description of Artificial Sequence Synthetic oligonucleotide
ctgaagnnnnnnnnnnnnnnnn

SEQ ID NO: 145 - DNA, 6 bp, Artificial Sequence
  Description of Artificial Sequence Synthetic oligonucleotide
gacgtc

SEQ ID NO: 146 - DNA, 9 bp, Artificial Sequence
  Description of Artificial Sequence Synthetic oligonucleotide
gacnnngtc

SEQ ID NO: 147 - DNA, 8 bp, Artificial Sequence
  Description of Artificial Sequence Synthetic oligonucleotide
gaagacnn

SEQ ID NO: 148 - DNA, 12 bp, Artificial Sequence
  Description of Artificial Sequence Synthetic oligonucleotide
nnnnnngtcttc

SEQ ID NO: 149 - DNA, 7 bp, Artificial Sequence
  Description of Artificial Sequence Synthetic oligonucleotide
gaatgcn

SEQ ID NO: 150 - DNA, 7 bp, Artificial Sequence
  Description of Artificial Sequence Synthetic oligonucleotide
ngcattc

SEQ ID NO: 151 - DNA, 4 bp, Artificial Sequence
  Description of Artificial Sequence Synthetic oligonucleotide
gatc

SEQ ID NO: 152 - DNA, 6 bp, Artificial Sequence
  Description of Artificial Sequence Synthetic oligonucleotide
gagctc

SEQ ID NO: 153 - DNA, 6 bp, Artificial Sequence
  Description of Artificial Sequence Synthetic oligonucleotide
grcgyc

SEQ ID NO: 154 - DNA, 6 bp, Artificial Sequence
  Description of Artificial Sequence Synthetic oligonucleotide
gwgcwc

SEQ ID NO: 155 - DNA, 6 bp, Artificial Sequence
  Description of Artificial Sequence Synthetic oligonucleotide
grgcyc

SEQ ID NO: 156 - DNA, 6 bp, Artificial Sequence
  Description of Artificial Sequence Synthetic oligonucleotide
gdgchc

SEQ ID NO: 157 - DNA, 6 bp, Artificial Sequence
  Description of Artificial Sequence Synthetic oligonucleotide
gaattc

SEQ ID NO: 158 - DNA, 6 bp, Artificial Sequence
  Description of Artificial Sequence Synthetic oligonucleotide
gatatc

SEQ ID NO: 159 - DNA, 10 bp, Artificial Sequence
  Description of Artificial Sequence Synthetic oligonucleotide
gacgcnnnnn

SEQ ID NO: 160 - DNA, 15 bp, Artificial Sequence
  Description of Artificial Sequence Synthetic oligonucleotide
nnnnnnnnnngcgtc

SEQ ID NO: 161 - DNA, 5 bp, Artificial Sequence
  Description of Artificial Sequence Synthetic oligonucleotide
gantc

SEQ ID NO: 162 - DNA, 13 bp, Artificial Sequence
  Description of Artificial Sequence Synthetic oligonucleotide
gaagannnnnnnn

SEQ ID NO: 163 - DNA, 12 bp, Artificial Sequence
  Description of Artificial Sequence Synthetic oligonucleotide
nnnnnnntcttc

SEQ ID NO: 164 - DNA, 9 bp, Artificial Sequence
  Description of Artificial Sequence Synthetic oligonucleotide
gagtcnnnn

SEQ ID NO: 165 - DNA, 10 bp, Artificial Sequence
  Description of Artificial Sequence Synthetic oligonucleotide
nnnnngactc

SEQ ID NO: 166 - DNA, 6 bp, Artificial Sequence
  Description of Artificial Sequence Synthetic oligonucleotide
gagctc

SEQ ID NO: 167 - DNA, 5 bp, Artificial Sequence
  Description of Artificial Sequence Synthetic oligonucleotide
gawtc

SEQ ID NO: 168 - DNA, 14 bp, Artificial Sequence
  Description of Artificial Sequence Synthetic oligonucleotide
nnnnnnnnngatgc

SEQ ID NO: 169 - DNA, 10 bp, Artificial Sequence
  Description of Artificial Sequence Synthetic oligonucleotide
gcatcnnnnn

SEQ ID NO: 170 - DNA, 10 bp, Artificial Sequence
  Description of Artificial Sequence Synthetic oligonucleotide
nnnnngatcc

SEQ ID NO: 171 - DNA, 9 bp, Artificial Sequence
  Description of Artificial Sequence Synthetic oligonucleotide
ggatcnnnn

SEQ ID NO: 172 - DNA, 11 bp, Artificial Sequence
  Description of Artificial Sequence Synthetic oligonucleotide
nnnnngagacc

SEQ ID NO: 173 - DNA, 7 bp, Artificial Sequence
  Description of Artificial Sequence Synthetic oligonucleotide
ggtctcn

SEQ ID NO: 174 - DNA, 10 bp, Artificial Sequence
  Description of Artificial Sequence Synthetic oligonucleotide
nnnnngagac

SEQ ID NO: 175 - DNA, 6 bp, Artificial Sequence
  Description of Artificial Sequence Synthetic oligonucleotide
gtctcn

SEQ ID NO: 176 - DNA, 10 bp, Artificial Sequence
  Description of Artificial Sequence Synthetic oligonucleotide
nnnngaagag

SEQ ID NO: 177 - DNA, 7 bp, Artificial Sequence
  Description of Artificial Sequence Synthetic oligonucleotide
ctcttcn

SEQ ID NO: 178 - DNA, 11 bp, Artificial Sequence
  Description of Artificial Sequence Synthetic oligonucleotide
nnnnngagacg

SEQ ID NO: 179 - DNA, 7 bp, Artificial Sequence
  Description of Artificial Sequence Synthetic oligonucleotide
cgtctcn

SEQ ID NO: 180 - DNA, 10 bp, Artificial Sequence
  Description of Artificial Sequence Synthetic oligonucleotide
nnnnnngagg

SEQ ID NO: 181 - DNA, 11 bp, Artificial Sequence
  Description of Artificial Sequence Synthetic oligonucleotide
cctcnnnnnnn

SEQ ID NO: 182 - DNA, 10 bp, Artificial Sequence
  Description of Artificial Sequence Synthetic oligonucleotide
nnnnngactc

SEQ ID NO: 183 - DNA, 9 bp, Artificial Sequence
  Description of Artificial Sequence Synthetic oligonucleotide
gagtcnnnn

SEQ ID NO: 184 - DNA, 13 bp, Artificial Sequence
  Description of Artificial Sequence Synthetic oligonucleotide
gcagcnnnnnnnn

SEQ ID NO: 185 - DNA, 17 bp, Artificial Sequence
  Description of Artificial Sequence Synthetic oligonucleotide
nnnnnnnnnnnngctgc

SEQ ID NO: 186 - DNA, 7 bp, Artificial Sequence
  Description of Artificial Sequence Synthetic oligonucleotide
gctnagc

SEQ ID NO: 187 - DNA, 6 bp, Artificial Sequence
  Description of Artificial Sequence Synthetic oligonucleotide
gcgcgc

SEQ ID NO: 188 - DNA, 4 bp, Artificial Sequence
  Description of Artificial Sequence Synthetic oligonucleotide
gcgc

SEQ ID NO: 189 - DNA, 5 bp, Artificial Sequence
  Description of Artificial Sequence Synthetic oligonucleotide
gcngc

SEQ ID NO: 190 - DNA, 6 bp, Artificial Sequence
  Description of Artificial Sequence Synthetic oligonucleotide
gccggc

SEQ ID NO: 191 - DNA, 6 bp, Artificial Sequence
  Description of Artificial Sequence Synthetic oligonucleotide
gccggc

SEQ ID NO: 192 - DNA, 6 bp, Artificial Sequence
  Description of Artificial Sequence Synthetic oligonucleotide
gctagc

SEQ ID NO: 193 - DNA, 8 bp, Artificial Sequence
  Description of Artificial Sequence Synthetic oligonucleotide
gcggccgc

SEQ ID NO: 194 - DNA, 10 bp, Artificial Sequence
  Description of Artificial Sequence Synthetic oligonucleotide
gcatcnnnnn

SEQ ID NO: 195 - DNA, 14 bp, Artificial Sequence
  Description of Artificial Sequence Synthetic oligonucleotide
nnnnnnnnngatgc

SEQ ID NO: 196 - DNA, 6 bp, Artificial Sequence
  Description of Artificial Sequence Synthetic oligonucleotide
gcatgc

SEQ ID NO: 197 - DNA, 8 bp, Artificial Sequence
  Description of Artificial Sequence Synthetic oligonucleotide
gcccgggc

SEQ ID NO: 198 - DNA, 4 bp, Artificial Sequence
  Description of Artificial Sequence Synthetic oligonucleotide
gcgg

SEQ ID NO: 199 - DNA, 17 bp, Artificial Sequence
  Description of Artificial Sequence Synthetic oligonucleotide
nnnnnnnnnnnngctgc

SEQ ID NO: 200 - DNA, 13 bp, Artificial Sequence
  Description of Artificial Sequence Synthetic oligonucleotide
gcagcnnnnnnnn

SEQ ID NO: 201 - DNA, 34 bp, Artificial Sequence; identical to SEQ ID NO: 14
  Description of Artificial Sequence Synthetic oligonucleotide

SEQ ID NO: 202 - DNA, 34 bp, Artificial Sequence; identical to SEQ ID NO: 13
  Description of Artificial Sequence Synthetic oligonucleotide

SEQ ID NO: 203 - DNA, 7 bp, Artificial Sequence
  Description of Artificial Sequence Synthetic oligonucleotide
ngcattc

SEQ ID NO: 204 - DNA, 7 bp, Artificial Sequence
  Description of Artificial Sequence Synthetic oligonucleotide
gaatgcn

SEQ ID NO: 205 - DNA, 14 bp, Artificial Sequence
  Description of Artificial Sequence Synthetic oligonucleotide
nnnnnnnngcaggt

SEQ ID NO: 206 - DNA, 10 bp, Artificial Sequence
  Description of Artificial Sequence Synthetic oligonucleotide
acctgcnnnn

SEQ ID NO: 207 - DNA, 15 bp, Artificial Sequence
  Description of Artificial Sequence Synthetic oligonucleotide
nnnnnnnnnngcgtc

SEQ ID NO: 208 - DNA, 10 bp, Artificial Sequence
  Description of Artificial Sequence Synthetic oligonucleotide
gacgcnnnnn

SEQ ID NO: 209 - DNA, 6 bp, Artificial Sequence
  Description of Artificial Sequence Synthetic oligonucleotide
ggtacc

SEQ ID NO: 210 - DNA, 5 bp, Artificial Sequence
  Description of Artificial Sequence Synthetic oligonucleotide
ggwcc

SEQ ID NO: 211 - DNA, 9 bp, Artificial Sequence
  Description of Artificial Sequence Synthetic oligonucleotide
ggatcnnnn

SEQ ID NO: 212 - DNA, 10 bp, Artificial Sequence
  Description of Artificial Sequence Synthetic oligonucleotide
nnnnngatcc

SEQ ID NO: 213 - DNA, 6 bp, Artificial Sequence
  Description of Artificial Sequence Synthetic oligonucleotide
gggccc

SEQ ID NO: 214 - DNA, 8 bp, Artificial Sequence
  Description of Artificial Sequence Synthetic oligonucleotide
ggcgcgcc

SEQ ID NO: 215 - DNA, 6 bp, Artificial Sequence
  Description of Artificial Sequence Synthetic oligonucleotide
ggatcc

SEQ ID NO: 216 - DNA, 6 bp, Artificial Sequence
  Description of Artificial Sequence Synthetic oligonucleotide
ggyrcc

SEQ ID NO: 217 - DNA, 7 bp, Artificial Sequence
  Description of Artificial Sequence Synthetic oligonucleotide
ggtctcn

SEQ ID NO: 218 - DNA, 11 bp, Artificial Sequence
  Description of Artificial Sequence Synthetic oligonucleotide
nnnnngagacc

SEQ ID NO: 219 - DNA, 7 bp, Artificial Sequence
  Description of Artificial Sequence Synthetic oligonucleotide
ggtnacc

SEQ ID NO: 220 - DNA, 14 bp, Artificial Sequence
  Description of Artificial Sequence Synthetic oligonucleotide
ggatgnnnnnnnnn

SEQ ID NO: 221 - DNA, 18 bp, Artificial Sequence
  Description of Artificial Sequence Synthetic oligonucleotide
nnnnnnnnnnnnncatcc

SEQ ID NO: 222 - DNA, 8 bp, Artificial Sequence
  Description of Artificial Sequence Synthetic oligonucleotide
ggccggcc

SEQ ID NO: 223 - DNA, 4 bp, Artificial Sequence
  Description of Artificial Sequence Synthetic oligonucleotide
ggcc

SEQ ID NO: 224 - DNA, 13 bp, Artificial Sequence
  Description of Artificial Sequence Synthetic oligonucleotide
ggtgannnnnnnn

SEQ ID NO: 225 - DNA, 12 bp, Artificial Sequence
  Description of Artificial Sequence Synthetic oligonucleotide
nnnnnnntcacc

SEQ ID NO: 226 - DNA, 6 bp, Artificial Sequence
  Description of Artificial Sequence Synthetic oligonucleotide
ggcgcc

SEQ ID NO: 227 - DNA, 6 bp, Artificial Sequence
  Description of Artificial Sequence Synthetic oligonucleotide
ggtacc

SEQ ID NO: 228 - DNA, 6 bp, Artificial Sequence
  Description of Artificial Sequence Synthetic oligonucleotide
ggcgcc

SEQ ID NO: 229 - DNA, 6 bp, Artificial Sequence
  Description of Artificial Sequence Synthetic oligonucleotide
ggnncc

SEQ ID NO: 230 - DNA, 5 bp, Artificial Sequence
  Description of Artificial Sequence Synthetic oligonucleotide
ggncc

SEQ ID NO: 231 - DNA, 6 bp, Artificial Sequence
  Description of Artificial Sequence Synthetic oligonucleotide
gtmkac

SEQ ID NO: 232 - DNA, 6 bp, Artificial Sequence
  Description of Artificial Sequence Synthetic oligonucleotide
gtgcac

SEQ ID NO: 233 - DNA, 22 bp, Artificial Sequence
  Description of Artificial Sequence Synthetic oligonucleotide
gtgcagnnnnnnnnnnnnnnnn

SEQ ID NO: 234 - DNA, 20 bp, Artificial Sequence
  Description of Artificial Sequence Synthetic oligonucleotide
nnnnnnnnnnnnnnctgcac

SEQ ID NO: 235 - DNA, 6 bp, Artificial Sequence
  Description of Artificial Sequence Synthetic oligonucleotide
gtctcn

SEQ ID NO: 236 - DNA, 10 bp, Artificial Sequence
  Description of Artificial Sequence Synthetic oligonucleotide
nnnnngagac

SEQ ID NO: 237 - DNA, 6 bp, Artificial Sequence
  Description of Artificial Sequence Synthetic oligonucleotide
gtatac

SEQ ID NO: 238 - DNA, 6 bp, Artificial Sequence
  Description of Artificial Sequence Synthetic oligonucleotide
gtyrac

SEQ ID NO: 239 - DNA, 6 bp, Artificial Sequence
  Description of Artificial Sequence Synthetic oligonucleotide
gttaac

SEQ ID NO: 240 - DNA, 5 bp, Artificial Sequence
  Description of Artificial Sequence Synthetic oligonucleotide
gtnac

SEQ ID NO: 241 - DNA, 8 bp, Artificial Sequence
  Description of Artificial Sequence Synthetic oligonucleotide
gtttaaac

SEQ ID NO: 242 - DNA, 4 bp, Artificial Sequence
  Description of Artificial Sequence Synthetic oligonucleotide
gtac

SEQ ID NO: 243 - DNA, 6 bp, Artificial Sequence
  Description of Artificial Sequence Synthetic oligonucleotide
gtcgac

SEQ ID NO: 244 - DNA, 12 bp, Artificial Sequence
  Description of Artificial Sequence Synthetic oligonucleotide
nnnnnngtcttc

SEQ ID NO: 245 - DNA, 8 bp, Artificial Sequence
  Description of Artificial Sequence Synthetic oligonucleotide
gaagacnn

SEQ ID NO: 246 - DNA, 6 bp, Artificial Sequence
  Description of Artificial Sequence Synthetic oligonucleotide
tacgta

SEQ ID NO: 247 - DNA, 6 bp, Artificial Sequence
  Description of Artificial Sequence Synthetic oligonucleotide
tccgga

SEQ ID NO: 248 - DNA, 6 bp, Artificial Sequence
  Description of Artificial Sequence Synthetic oligonucleotide
tcatga

SEQ ID NO: 249 - DNA, 6 bp, Artificial Sequence
  Description of Artificial Sequence Synthetic oligonucleotide
tcgcga

SEQ ID NO: 250 - DNA, 4 bp, Artificial Sequence
  Description of Artificial Sequence Synthetic oligonucleotide
tcga

SEQ ID NO: 251 - DNA, 6 bp, Artificial Sequence
  Description of Artificial Sequence Synthetic oligonucleotide
tctaga

SEQ ID NO: 252 - DNA, 12 bp, Artificial Sequence
  Description of Artificial Sequence Synthetic oligonucleotide
nnnnnnntcacc

SEQ ID NO: 253 - DNA, 13 bp, Artificial Sequence
  Description of Artificial Sequence Synthetic oligonucleotide
ggtgannnnnnnn

SEQ ID NO: 254 - DNA, 12 bp, Artificial Sequence
  Description of Artificial Sequence Synthetic oligonucleotide
nnnnnnntcttc

SEQ ID NO: 255 - DNA, 13 bp, Artificial Sequence
  Description of Artificial Sequence Synthetic oligonucleotide
gaagannnnnnnn

SEQ ID NO: 256 - DNA, 6 bp, Artificial Sequence
  Description of Artificial Sequence Synthetic oligonucleotide
tgcgca

SEQ ID NO: 257 - DNA, 6 bp, Artificial Sequence
  Description of Artificial Sequence Synthetic oligonucleotide
tggcca

SEQ ID NO: 258 - DNA, 6 bp, Artificial Sequence
  Description of Artificial Sequence Synthetic oligonucleotide
tgatca

SEQ ID NO: 259 - DNA, 6 bp, Artificial Sequence
  Description of Artificial Sequence Synthetic oligonucleotide
tgtaca

SEQ ID NO: 260 - DNA, 6 bp, Artificial Sequence
  Description of Artificial Sequence Synthetic oligonucleotide
tttaaa

SEQ ID NO: 261 - DNA, 6 bp, Artificial Sequence
  Description of Artificial Sequence Synthetic oligonucleotide
ttcgaa

SEQ ID NO: 262 - DNA, 4 bp, Artificial Sequence
  Description of Artificial Sequence Synthetic oligonucleotide
ttaa

SEQ ID NO: 263 - DNA, 8 bp, Artificial Sequence
  Description of Artificial Sequence Synthetic oligonucleotide
ttaattaa

SEQ ID NO: 264 - DNA, 62 bp, Human immunodeficiency virus
tctctggttagaccagatctgagcctgggagctctctggctaactagggaacccactgctta

SEQ ID NO: 265 - DNA, 62 bp, Human immunodeficiency virus
taagcagtgggttccctagttagccagagagctcccaggctcagatctggtctaaccagaga

SEQ ID NO: 266 - DNA, 20 bp, Human immunodeficiency virus
tctctggttagaccagatct

SEQ ID NO: 267 - DNA, 22 bp, Human immunodeficiency virus
taagcagtgggttccctagtta

SEQ ID NO: 268 - DNA, 20 bp, Human immunodeficiency virus
gagcctgggagctctctggc

SEQ ID NO: 269 - DNA, 303 bp, Artificial Sequence
  Description of Artificial Sequence Synthetic polynucleotide
gctagcgccgccaccatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagcacatcagaacagtcagactcatcaagcttctctatcaaagcaacccacctcccaaccccgaggggacccgacaggcccgaaggaaatcgatgctggttctagagcaggactcgag

SEQ ID NO: 270 - DNA, 370 bp, Artificial Sequence
  Description of Artificial Sequence Synthetic polynucleotide
atcgatgctggttctagagcaggactcgagatgggcataacgataaaaaagagcactgccgagcaggttctaaggaaagcctacgaagctgcggcatcagacgatgtgtttcttgaggactggatctttctggctactagtctccgcgaggtcgatgcccctagaacatacaccgccgcgctagtcaccgctttgcttgcacgtgcttgtgacgatcgagttgatcccaggagcattaaagaaaaatatgacgatagagcgttttccttaaggacgctttgtcatggggtagttgtaccaatgtcggttgagttaggatttgaccttggcgcgacaggaagagagcctataaacaatcagcccttttttc

SEQ ID NO: 271 - DNA, 400 bp, Artificial Sequence
  Description of Artificial Sequence Synthetic polynucleotide
gagagcctataaacaatcagcccttttttcgctacgatcagtacagtgaaatcgtcagggtccaaaccaaagccagaccgtatttggaccgagtatcgtcagccttagccagggtcgatgaagaggattattcgacggaagagagctttcgcgcattagtagcggtgctcgcggtttgtatcagtgttgcgaacaagaaacaaagagttgcagtagggtcagctattgttgaagcaagtttaatcgcagagacacaaagctttgtggtaagcggccacgacgttcctcggaagttgcaagcctgtgtggcagccggattggacatggtatatagtgaagtcgtatcgcgacgtataaatgacccgtcccgtgattttcctggggatgttcaggtaatctt

SEQ ID NO: 272 - DNA, 400 bp, Artificial Sequence
  Description of Artificial Sequence Synthetic polynucleotide
tgattttcctggggatgttcaggtaatcttagatggagacccattgctgacagtcgaggtacgtggtaagtctgtgagctgggagggtctcgaacaatttgtgtcttcagcaacgtacgcgggttttaggcgcgtggcactaatggtggatgcggcttcccacgtgtcactgatgtctgctgatgacctaacttcagctttggagcggaaatatgagtgtattgtcaaggtaaatgagagcgtcagttcctttctccgagacgtatttgtctggtctccaagggatgtgcatagtattctatcagcttttcccgaagcaatgtatagacggatgattgaaatagaagtacgggaaccggaactggacagatgggctgagatatttccagaaactgatatc

SEQ ID NO: 273 - DNA, 106 bp, Artificial Sequence
  Description of Artificial Sequence Synthetic polynucleotide
gaacctgaccccagaccaggtagtcgcaatcgcgtcacatgacgggggaaagcaagccctggaaaccgtgcaaaggttgttgccggtcctttgtcaagaccacggc

SEQ ID NO: 274 - protein, 35 aa, Artificial Sequence
  Description of Artificial Sequence Synthetic polypeptide
NLTPDQVVAIASHDGGKQALETVQRLLPVLCQDHG

SEQ ID NO: 275 - DNA, 102 bp, Artificial Sequence
  Description of Artificial Sequence Synthetic polynucleotide
cttacaccggagcaagtcgtggccattgcaagcaatgggggtggcaaacaggctcttgagacggttcagagacttctcccagttctctgtcaagcccacggg

SEQ ID NO: 276 - protein, 34 aa, Artificial Sequence
  Description of Artificial Sequence Synthetic polypeptide
LTPEQVVAIASNGGGKQALETVQRLLPVLCQAHG

SEQ ID NO: 277 - DNA, 102 bp, Artificial Sequence
  Description of Artificial Sequence Synthetic polynucleotide
ctgactcccgatcaagttgtagcgattgcgtcgcatgacggagggaaacaagcattggagactgtccaacggctccttcccgtgttgtgtcaagcccacggt

SEQ ID NO: 278 - protein, 34 aa, Artificial Sequence
  Description of Artificial Sequence Synthetic polypeptide
LTPDQVVAIASHDGGKQALETVQRLLPVLCQAHG

SEQ ID NO: 279 - DNA, 102 bp, Artificial Sequence
  Description of Artificial Sequence Synthetic polynucleotide
ttgacgcctgcacaagtggtcgccatcgcctcgaatggcggcggtaagcaggcgctggaaacagtacagcgcctgctgcctgtactgtgccaggatcatgga

SEQ ID NO: 280 - protein, 34 aa, Artificial Sequence
  Description of Artificial Sequence Synthetic polypeptide
LTPAQVVAIASNGGGKQALETVQRLLPVLCQDHG

SEQ ID NO: 281 - DNA, 102 bp, Artificial Sequence
  Description of Artificial Sequence Synthetic polynucleotide
ttgacgcctgcacaagtggtcgccatcgcctcgaattcgggcggtaagcaggcgctggaaacagtacagcgcctgctgcctgtactgtgccaggatcatgga

SEQ ID NO: 282 - protein, 34 aa, Artificial Sequence
  Description of Artificial Sequence Synthetic polypeptide
LTPAQVVAIASNSGGKQALETVQRLLPVLCQDHG

SEQ ID NO: 283 - DNA, 102 bp, Artificial Sequence
  Description of Artificial Sequence Synthetic polynucleotide
ctgaccccagaccaggtagtcgcaatcgcgaacaataatgggggaaagcaagccctggaaaccgtgcaaaggttgttgccggtcctttgtcaagaccacggc

SEQ ID NO: 284 - protein, 34 aa, Artificial Sequence
  Description of Artificial Sequence Synthetic polypeptide
LTPDQVVAIANNNGGKQALETVQRLLPVLCQDHG

SEQ ID NO: 285 - DNA, 102 bp, Artificial Sequence
  Description of Artificial Sequence Synthetic polynucleotide
ctgaccccagaccaggtagtcgcaatcgcgaacaattcggggggaaagcaagccctggaaaccgtgcaaaggttgttgccggtcctttgtcaagaccacggc

SEQ ID NO: 286 - protein, 34 aa, Artificial Sequence
  Description of Artificial Sequence Synthetic polypeptide
LTPDQVVAIANNSGGKQALETVQRLLPVLCQDHG

SEQ ID NO: 287 - DNA, 102 bp, Artificial Sequence
  Description of Artificial Sequence Synthetic polynucleotide
cttacaccggagcaagtcgtggccattgcaaataataacggtggcaaacaggctcttgagacggttcagagacttctcccagttctctgtcaagcccacggg

SEQ ID NO: 288 - protein, 34 aa, Artificial Sequence
  Description of Artificial Sequence Synthetic polypeptide
LTPEQVVAIANNNGGKQALETVQRLLPVLCQAHG

SEQ ID NO: 289 - DNA, 102 bp, Artificial Sequence
  Description of Artificial Sequence Synthetic polynucleotide
ctgactcccgatcaagttgtagcgattgcgtccaacggtggagggaaacaagcattggagactgtccaacggctccttcccgtgttgtgtcaagcccacggt

SEQ ID NO: 290 - protein, 34 aa, Artificial Sequence
  Description of Artificial Sequence Synthetic polypeptide
LTPDQVVAIASNGGGKQALETVQRLLPVLCQAHG

SEQ ID NO: 291 - DNA, 102 bp, Artificial Sequence
  Description of Artificial Sequence Synthetic polynucleotide
ctgaccccagaccaggtagtcgcaatcgcgtcgaacattgggggaaagcaagccctggaaaccgtgcaaaggttgttgccggtcctttgtcaagaccacggc

SEQ ID NO: 292 - protein, 34 aa, Artificial Sequence
  Description of Artificial Sequence Synthetic polypeptide
LTPDQVVAIASNIGGKQALETVQRLLPVLCQDHG

SEQ ID NO: 293 - DNA, 102 bp, Artificial Sequence
  Description of Artificial Sequence Synthetic polynucleotide
ctgactcccgatcaagttgtagcgattgcgtcgaacattggagggaaacaagcattggagactgtccaacggctccttcccgtgttgtgtcaagcccacggt

SEQ ID NO: 294 - protein, 34 aa, Artificial Sequence
  Description of Artificial Sequence Synthetic polypeptide
LTPDQVVAIASNIGGKQALETVQRLLPVLCQAHG

SEQ ID NO: 295 - DNA, 102 bp, Artificial Sequence
  Description of Artificial Sequence Synthetic polynucleotide
ttgacgcctgcacaagtggtcgccatcgccagccatgatggcggtaagcaggcgctggaaacagtacagcgcctgctgcctgtactgtgccaggatcatgga

SEQ ID NO: 296 - protein, 34 aa, Artificial Sequence
  Description of Artificial Sequence Synthetic polypeptide
LTPAQVVAIASHDGGKQALETVQRLLPVLCQDHG

SEQ ID NO: 297 - DNA, 102 bp, Artificial Sequence
  Description of Artificial Sequence Synthetic polynucleotide
ctgaccccagaccaggtagtcgcaatcgcgtcacatgacgggggaaagcaagccctggaaaccgtgcaaaggttgttgccggtcctttgtcaagaccacggc

SEQ ID NO: 298 - protein, 34 aa, Artificial Sequence
  Description of Artificial Sequence Synthetic polypeptide
LTPDQVVAIASHDGGKQALETVQRLLPVLCQDHG

SEQ ID NO: 299 - DNA, 102 bp, Artificial Sequence
  Description of Artificial Sequence Synthetic polynucleotide
cttacaccggagcaagtcgtggccattgcaagcaacatcggtggcaaacaggctcttgagacggttcagagacttctcccagttctctgtcaagcccacggg

SEQ ID NO: 300 - protein, 34 aa, Artificial Sequence
  Description of Artificial Sequence Synthetic polypeptide
LTPEQVVAIASNIGGKQALETVQRLLPVLCQAHG

SEQ ID NO: 301 - DNA, 102 bp, Artificial Sequence
  Description of Artificial Sequence Synthetic polynucleotide
ctgactcccgatcaagttgtagcgattgcgaataacaatggagggaaacaagcattggagactgtccaacggctccttcccgtgttgtgtcaagcccacggt

SEQ ID NO: 302 - protein, 34 aa, Artificial Sequence
  Description of Artificial Sequence Synthetic polypeptide
LTPDQVVAIANNNGGKQALETVQRLLPVLCQAHG

SEQ ID NO: 303 - DNA, 102 bp, Artificial Sequence
  Description of Artificial Sequence Synthetic polynucleotide
ttgacgcctgcacaagtggtcgccatcgcctccaatattggcggtaagcaggcgctggaaacagtacagcgcctgctgcctgtactgtgccaggatcatgga

SEQ ID NO: 304 - protein, 34 aa, Artificial Sequence
  Description of Artificial Sequence Synthetic polypeptide
LTPAQVVAIASNIGGKQALETVQRLLPVLCQDHG

SEQ ID NO: 305 - DNA, 102 bp, Artificial Sequence
  Description of Artificial Sequence Synthetic polynucleotide
ctgaccccagaccaggtagtcgcaatcgcgtcaaacggagggggaaagcaagccctggaaaccgtgcaaaggttgttgccggtcctttgtcaagaccacggc

SEQ ID NO: 306 - protein, 34 aa, Artificial Sequence
  Description of Artificial Sequence Synthetic polypeptide
LTPDQVVAIASNGGGKQALETVQRLLPVLCQDHG

SEQ ID NO: 307 - DNA, 102 bp, Artificial Sequence
  Description of Artificial Sequence Synthetic polynucleotide
cttacaccggagcaagtcgtggccattgcatcccacgacggtggcaaacaggctcttgagacggttcagagacttctcccagttctctgtcaagcccacggg

SEQ ID NO: 308 - protein, 34 aa, Artificial Sequence
  Description of Artificial Sequence Synthetic polypeptide
LTPEQVVAIASHDGGKQALETVQRLLPVLCQAHG

SEQ ID NO: 309 - DNA, 102 bp, Artificial Sequence
  Description of Artificial Sequence Synthetic polynucleotide
cttacaccggagcaagtcgtggccattgcatccaattcgggtggcaaacaggctcttgagacggttcagagacttctcccagttctctgtcaagcccacggg

SEQ ID NO: 310 - protein, 34 aa, Artificial Sequence
  Description of Artificial Sequence Synthetic polypeptide
LTPEQVVAIASNSGGKQALETVQRLLPVLCQAHG

SEQ ID NO: 311 - DNA, 106 bp, Artificial Sequence
  Description of Artificial Sequence Synthetic polynucleotide
gaacctgaccccagaccaggtagtcgcaatcgcgtcgaacattgggggaaagcaagccctggaaaccgtgcaaaggttgttgccggtcctttgtcaagaccacggc

SEQ ID NO: 312 - protein, 35 aa, Artificial Sequence
  Description of Artificial Sequence Synthetic polypeptide
NLTPDQVVAIASNIGGKQALETVQRLLPVLCQDHG

SEQ ID NO: 313 - DNA, 102 bp, Artificial Sequence
  Description of Artificial Sequence Synthetic polynucleotide
ttgacgcctgcacaagtggtcgccatcgccaacaacaacggcggtaagcaggcgctggaaacagtacagcgcctgctgcctgtactgtgccaggatcatgga

SEQ ID NO: 314 - protein, 34 aa, Artificial Sequence
  Description of Artificial Sequence Synthetic polypeptide
LTPAQVVAIANNNGGKQALETVQRLLPVLCQDHG

SEQ ID NO: 315 - DNA, 102 bp, Artificial Sequence; identical to SEQ ID NO: 307
  Description of Artificial Sequence Synthetic polynucleotide

SEQ ID NO: 316 - protein, 34 aa, Artificial Sequence; identical to SEQ ID NO: 308
  Description of Artificial Sequence Synthetic polypeptide

SEQ ID NO: 317 - DNA, 102 bp, Artificial Sequence; identical to SEQ ID NO: 277
  Description of Artificial Sequence Synthetic polynucleotide

SEQ ID NO: 318 - protein, 34 aa, Artificial Sequence; identical to SEQ ID NO: 278
  Description of Artificial Sequence Synthetic polypeptide

SEQ ID NO: 319 - DNA, 3421 bp, Artificial Sequence
  Description of Artificial Sequence Synthetic polynucleotide
gctagcgccgccaccgatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagcaggagcacatcagaacagtcagactcatcaagcttctctatcaaagcaacccacctcccaaccccgaggggacccgacaggcccgaaggaaatcgataagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgtcacatgacgggggaaagcaagccctggaaaccgtgcaaaggttgttgccggtcctttgtcaagaccacggccttacaccggagcaagtcgtggccattgcaagcaatgggggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaattcggggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaattcgggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgaacattggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgtcaaacggagggggaaagcaagccctggaaaccgtgcaaaggttgttgccggtcctttgtcaagaccacggccttacaccggagcaagtcgtggccattgcatccaattcgggtggcaaacaggctcttgagacggttcagagacttctcccagttctctgtcaagcccacgggctgacacccgaacaggtggtcgccattgcttctaatgggggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttgatatc

SEQ ID NO: 320 - protein, 1138 aa, Artificial Sequence
  Description of Artificial Sequence Synthetic polypeptide
ASAATMEPVDPRLEPWKHPGSQPKTACTNCYCKKCCFHCQVCFITKALGISYGRKKRRQRRRAHQNSQTHQASLSKQPTSQPRGDPTGPKEIDKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNSGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNSGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNSGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFDI

SEQ ID NO: 321 - DNA, 3618 bp, Artificial Sequence
  Description of Artificial Sequence Synthetic polynucleotide
gctagcgccgccaccatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagcacatcagaacagtcagactcatcaagcttctctatcaaagcaacccacctcccaaccccgaggggacccgacaggcccgaaggaaatcgataagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgacacccgaacaggtggtcgccattgcttctaacatcggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttgatatc

SEQ ID NO: 322 - protein, 1206 aa, Artificial Sequence
  Description of Artificial Sequence Synthetic polypeptide
ASAATMEPVDPRLEPWKHPGSQPKTACTNCYCKKCCFHCQVCFITKALGISYGRKKRRQRRRAHQNSQTHQASLSKQPTSQPRGDPTGPKEIDKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFDI

SEQ ID NO: 323 - DNA, 3906 bp, Artificial Sequence
  Description of Artificial Sequence Synthetic polynucleotide
gctagcgccgccaccatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagcacatcagaacagtcagactcatcaagcttctctatcaaagcaacccacctcccaaccccgaggggacccgacaggcccgaaggaaatcgataagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgtcacatgacgggggaaagcaagccctggaaaccgtgcaaaggttgttgccggtcctttgtcaagaccacggccttacaccggagcaagtcgtggccattgcaagcaatgggggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgaacaattcggggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaattcgggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtcgaacattggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgcctccaatattggcggtaagcaggcgctggaaacagtacagcgcctgctgcctgtactgtgccaggatcatggactgaccccagaccaggtagtcgcaatcgcgtcaaacggagggggaaagcaagccctggaaaccgtgcaaaggttgttgccggtcctttgtcaagaccacggccttacaccggagcaagtcgtggccattgcatccaattcgggtggcaaacaggctcttgagacggttcagagacttctcccagttctctgtcaagcccacgggctgacacccgaacaggtggtcgccattgcttctaatgggggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatccctcgagatgggcataacgataaaaaagagcactgccgagcaggttctaaggaaagcctacgaagctgcggcatcagacgatgtgtttcttgaggactggatctttctggctactagtctccgcgaggtcgatgcccctagaacatacaccgccgcgctagtcaccgctttgcttgcacgtgcttgtgacgatcgagttgatcccaggagcattaaagaaaaatatgacgatagagcgttttccttaaggacgctttgtcatggggtagttgtaccaatgtcggttgagttaggatttgaccttggcgcgacaggaagagagcctataaacaatcagcccttttttcgctacgatcagtacagtgaaatcgtcagggtccaaaccaaagccagaccgtatttggaccgagtatcgtcagccttagccagggtcgatgaagaggattattcgacggaagagagctttcgcgcattagtagcggtgctcgcggtttgtatcagtgttgcgaacaagaaacaaagagttgcagtagggtcagctattgttgaagcaagtttaatcgcagagacacaaagctttgtggtaagcggccacgacgttcctcggaagttgcaagcctgtgtggcagccggattggacatggtatatagtgaagtcgtatcgcgacgtataaatgacccgtcccgtgattttcctggggatgttcaggtaatcttagatggagacccattgctgacagtcgaggtacgtggtaagtctgtgagctgggagggtctcgaacaatttgtgtcttcagcaacgtacgcgggttttaggcgcgtggcactaatggtggatgcggcttcccacgtgtcactgatgtctgctgatgacctaacttcagctttggagcggaaatatgagtgtattgtcaaggtaaatgagagcgtcagttcctttctccgagacgtatttgtctggtctccaagggatgtgcatagtattctatcagcttttcccgaagcaatgtatagacggatgattgaaatagaagtacgggaaccggaactggacagatgggctgagatatttccagaaactgatatc

SEQ ID NO: 324 - protein, 1302 aa, Artificial Sequence
  Description of Artificial Sequence Synthetic polypeptide
ASAATMEPVDPRLEPWKHPGSQPKTACTNCYCKKCCFHCQVCFITKALGISYGRKKRRQRRRAHQNSQTHQASLSKQPTSQPRGDPTGPKEIDKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIANNSGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNSGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNSGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVAGSLEMGITIKKSTAEQVLRKAYEAAASDDVFLEDWIFLATSLREVDAPRTYTAALVTALLARACDDRVDPRSIKEKYDDRAFSLRTLCHGVVVPMSVELGFDLGATGREPINNQPFFRYDQYSEIVRVQTKARPYLDRVSSALARVDEEDYSTEESFRALVAVLAVCISVANKKQRVAVGSAIVEASLIAETQSFVVSGHDVPRKLQACVAAGLDMVYSEVVSRRINDPSRDFPGDVQVILDGDPLLTVEVRGKSVSWEGLEQFVSSATYAGFRRVALMVDAASHVSLMSADDLTSALERKYECIVKVNESVSSFLRDVFVWSPRDVHSILSAFPEAMYRRMIEIEVREPELDRWAEIFPETDI

SEQ ID NO: 325 - DNA, 4110 bp, Artificial Sequence
  Description of Artificial Sequence Synthetic polynucleotide
gctagcgccgccaccatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagcacatcagaacagtcagactcatcaagcttctctatcaaagcaacccacctcccaaccccgaggggacccgacaggcccgaaggaaatcgataagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacctgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaagcaacatcggtggcaaacaggctcttgagacggttcagagacttctcccagttctctgtcaagcccacgggctgactcccgatcaagttgtagcgattgcgaataacaatggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgaacaataatgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggccttacaccggagcaagtcgtggccattgcatcccacgacggtggcaaacaggctcttgagacggttcagagacttctcccagttctctgtcaagcccacgggctgactcccgatcaagttgtagcgattgcgtcgcatgacggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgaccccagaccaggtagtcgcaatcgcgtcgaacattgggggaaagcaagccctggaaaccgtgcaaaggttgttgccggtcctttgtcaagaccacggccttacaccggagcaagtcgtggccattgcaaataataacggtggcaaacaggctcttgagacggttcagagacttctcccagttctctgtcaagcccacgggctgactcccgatcaagttgtagcgattgcgtccaacggtggagggaaacaagcattggagactgtccaacggctccttcccgtgttgtgtcaagcccacggtttgacgcctgcacaagtggtcgccatcgcctcgaatggcggcggtaagcaggcgctggaaacagtacagcgcctgctgcctgtactgtgccaggatcatggactgacacccgaacaggtggtcgccattgcttctaacatcggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatccctcgagatgggcataacgataaaaaagagcactgccgagcaggttctaaggaaagcctacgaagctgcggcatcagacgatgtgtttcttgaggactggatctttctggctactagtctccgcgaggtcgatgcccctagaacatacaccgccgcgctagtcaccgctttgcttgcacgtgcttgtgacgatcgagttgatcccaggagcattaaagaaaaatatgacgatagagcgttttccttaaggacgctttgtcatggggtagttgtaccaatgtcggttgagttaggatttgaccttggcgcgacaggaagagagcctataaacaatcagcccttttttcgctacgatcagtacagtgaaatcgtcagggtccaaaccaaagccagaccgtatttggaccgagtatcgtcagccttagccagggtcgatgaagaggattattcgacggaagagagctttcgcgcattagtagcggtgctcgcggtttgtatcagtgttgcgaacaagaaacaaagagttgcagtagggtcagctattgttgaagcaagtttaatcgcagagacacaaagctttgtggtaagcggccacgacgttcctcggaagttgcaagcctgtgtggcagccggattggacatggtatatagtgaagtcgtatcgcgacgtataaatgacccgtcccgtgattttcctggggatgttcaggtaatcttagatggagacccattgctgacagtcgaggtacgtggtaagtctgtgagctgggagggtctcgaacaatttgtgtcttcagcaacgtacgcgggttttaggcgcgtggcactaatggtggatgcggcttcccacgtgtcactgatgtctgctgatgacctaacttcagctttggagcggaaatatgagtgtattgtcaaggtaaatgagagcgtcagttcctttctccgagacgtatttgtctggtctccaagggatgtgcatagtattctatcagcttttcccgaagcaatgtatagacggatgattgaaatagaagtacgggaaccggaactggacagatgggctgagatatttccagaaactgatatc

SEQ ID NO: 326 - protein, 1370 aa, Artificial Sequence
  Description of Artificial Sequence Synthetic polypeptide
ASAATMEPVDPRLEPWKHPGSQPKTACTNCYCKKCCFHCQVCFITKALGISYGRKKRRQRRRAHQNSQTHQASLSKQPTSQPRGDPTGPKEIDKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGSLEMGITIKKSTAEQVLRKAYEAAASDDVFLEDWIFLATSLREVDAPRTYTAALVTALLARACDDRVDPRSIKEKYDDRAFSLRTLCHGVVVPMSVELGFDLGATGREPINNQPFFRYDQYSEIVRVQTKARPYLDRVSSALARVDEEDYSTEESFRALVAVLAVCISVANKKQRVAVGSAIVEASLIAETQSFVVSGHDVPRKLQACVAAGLDMVYSEVVSRRINDPSRDFPGDVQVILDGDPLLTVEVRGKSVSWEGLEQFVSSATYAGFRRVALMVDAASHVSLMSADDLTSALERKYECIVKVNESVSSFLRDVFVWSPRDVHSILSAFPEAMYRRMIEIEVREPELDRWAEIFPETDI

SEQ ID NO: 327 - DNA, 120 bp, Artificial Sequence
  Description of Artificial Sequence Synthetic polynucleotide
cctcagatgctgcatataagcagctgctttttgcctgtactgggtctctctggttagaccagatctgagcctgggagctctctggctagctagggaacccactgcttaagcctcaataaa

SEQ ID NO: 328 - DNA, 120 bp, Artificial Sequence
  Description of Artificial Sequence Synthetic polynucleotide
cctcagatgctgcatataggcagctgctttttgcctgtactgggtctctctggttagaccagatctgagcctgggagctctctggctagctagggaacccactgcttaagcctcaataaa

SEQ ID NO: 329 - DNA, 120 bp, Human immunodeficiency virus; identical to SEQ ID NO: 327

SEQ ID NO: 330 - DNA, 60 bp, Human immunodeficiency virus
tctctggttagaccatctgagcctgggagctctctggctaactagggaacccactgctta

SEQ ID NO: 331 - protein, 8 aa, Artificial Sequence
  Description of Artificial Sequence Synthetic peptide
DYKDDDDK

SEQ ID NO: 332 - DNA, 14 bp, Human immunodeficiency virus
tctctggttagacc

SEQ ID NO: 333 - DNA, 22 bp, Human immunodeficiency virus
tagctagggaacccactgctta

SEQ ID NO: 334 - DNA, 19 bp, Human immunodeficiency virus
gagcctggagctctctggc

SEQ ID NO: 335 - DNA, 18 bp, Human immunodeficiency virus
agcctggagctctctggc

SEQ ID NO: 336 - DNA, 14 bp, Artificial Sequence
  Description of Artificial Sequence Synthetic oligonucleotide
tataaataccttct

SEQ ID NO: 337 - DNA, 16 bp, Artificial Sequence
  Description of Artificial Sequence Synthetic oligonucleotide
ctctggttagaccaga

SEQ ID NO: 338 - DNA, 18 bp, Artificial Sequence
  Description of Artificial Sequence Synthetic oligonucleotide
ctctggttagaccagatc

SEQ ID NO: 339 - DNA, 16 bp, Artificial Sequence
  Description of Artificial Sequence Synthetic oligonucleotide
aagcagtgggttccct

SEQ ID NO: 340 - DNA, 20 bp, Artificial Sequence
  Description of Artificial Sequence Synthetic oligonucleotide
aagcagtgggttccctagtt

(19) United States
(12) Patent Application Publication
(10) Pub. No.: US 2015/0104873 A1
(43) Pub. Date: Apr. 16, 2015

(54) Genome Surgery with Paired, Permeant Endonuclease Construct

(71) Applicant: UNIVERSITY OF NEVADA, LAS VEGAS, LAS VEGAS, NV (US)

(72) Inventors: CHRISTY STRONG, LAS VEGAS, NV (US); MARTIN SCHILLER, HENDERSON, NV (US)

(21) Appl. No.: 13/937,944

(22) Filed: Jul. 9, 2013

Related U.S. Application Data
(60) Provisional application No. 61/670,263, filed on Jul. 11, 2012.

(51) Int. Cl. C12N 15/63; C12N 15/85

(52) U.S. Cl. CPC C12N 15/63; C12N 15/85
USPC 435/455; 435/471; 435/320.1

(57) ABSTRACT

A chemical tool and its use in genome surgery includes P2E2 constructs of in, order, a cell penetration component, a DNA binding component and a restriction endonuclease. The method for performing genome surgery includes:
a) providing one or more recombinant of the P2E2 constructs;
b) penetrating a cell with the recombinant P2E2 protein construct;
c) forming a protein product in the cell by the processes of transcription and translation or by direct introduction of the P2E2 protein construct to the cell;
d) attaching the protein product of the P2E2 construct to one or more targeted genomic sequences within the cell; and
e) the endonuclease of the P2E2 construct cutting both strands of the genome at target locations.

### RELATED APPLICATION DATA

This application claims priority from U.S. provisional Patent Application 61/670,263, filed 11 Jul. 2012.

### BACKGROUND OF THE INVENTION

1. Field of the Invention

The present invention relates to the field of genome surgery and novel restricting enzymes used in such surgery.

2. Background of the Art

### Gene Therapy

Gene therapy is a rapidly growing field of medicine in which genes are introduced into the body to treat diseases. Genes are the fundamental unit of inheritance and provide the basic biological code for determining a cell's specific functions. Mutations, or minor changes in genes can impart dysfunction and disease. Gene therapy seeks to provide genes or corresponding protein coding regions that correct or supplant the disease-controlling functions of cells that are not, in essence, doing their job correctly. Somatic gene therapy introduces therapeutic genes at the tissue or cellular level to treat a specific individual. Germ-line gene therapy inserts genes into reproductive cells or possibly into embryos to correct genetic defects that could be passed on to future generations. Initially conceived as an approach for treating inherited diseases, like cystic fibrosis and Huntington's disease, the scope of potential gene therapies has grown to include treatments for cancers, arthritis, and infectious diseases. Although gene therapy testing in humans has advanced rapidly, many questions surround its use. For example, some scientists are concerned that the therapeutic genes themselves may cause disease.

Gene therapy has grown out of the science of genetics or how heredity works. Scientists know that life begins in a cell, the basic building block of all multicellular organisms. Humans, for instance, are made up of trillions of cells, each performing a specific function. Within the cell's nucleus (a compartment in a cell that regulates the majority of its chemical functions) are pairs of chromosomes. These thread-like structures are each made up of a single molecule of DNA (deoxyribonucleic acid), which carries the blueprint of life in the form of codes, or genes, that determine inherited characteristics.

A DNA molecule looks like two ladders with one of the sides taken off both and then twisted around each other. The rungs of these ladders meet (resulting in a spiral staircase-like structure) and are called base pairs. Base pairs are made up of nitrogenous bases arranged in specific sequences of adenine, cytosine, guanosine, and thymidine. Millions of these base pairs, or sequences, can make up a single gene, specifically defined as a segment of the chromosome that contains a unit of hereditary information. The gene or combination of genes formed by these base pairs ultimately direct an organism's growth and characteristics through the production of certain chemicals, primarily proteins, which carry out most of the body's chemical functions and biological reactions.

Scientists have long known that alterations in genes present within cells can cause inherited diseases like cystic fibrosis, sickle-cell anemia, and hemophilia. Similarly, errors in the total number of chromosomes can cause conditions such as Down syndrome or Turner's syndrome. As the study of genetics advanced, however, scientists learned that an altered genetic sequence also can make people more susceptible to diseases, like atherosclerosis, cancer, and even schizophrenia. These diseases have a genetic component, but also are influenced by environmental factors (like diet and lifestyle). The objective of gene therapy is to treat diseases by introducing functional genes into the body to alter the cells involved in the disease process by either replacing missing genes or providing copies of functioning genes to replace nonfunctioning ones. The inserted genes can be naturally-occurring genes that produce the desired effect or may be genetically engineered (or altered) genes.

Scientists have known how to manipulate a gene's structure in the laboratory since the early 1970s through a process called gene cloning. The process involves removing a fragment of DNA containing the specific genetic sequence desired, and then inserting it into the DNA of plasmid vector that controls production of the gene product or is designed to interfere with endogenous genes. The resultant product is called a recombinant DNA construct and the process is called genetic engineering. There are basically two types of gene therapy. Germ-line gene therapy introduces genes into reproductive cells (sperm and eggs) or someday possibly into embryos in hopes of correcting genetic abnormalities that could be passed on to future generations. Most of the current work in applying gene therapy, however, has been in the realm of somatic gene therapy. In this type of gene therapy, therapeutic genes are inserted into tissue or cells to produce a naturally occurring protein or substance that is lacking or disfunctional in an individual patient.

### Viral Delivery Vectors

In both types of therapy, scientists need a means to deliver either the entire gene or a recombinant DNA to the cell's nucleus, where the chromosomes (the packaged DNA) reside. There are several different ways of introducing recombinant DNA into cells. One of the first and most popular delivery vectors developed were viruses because they invade cells as part of the natural infection process. Viruses have the potential to be excellent delivery vectors because they have a specific relationship with the host in that they colonize certain cell types and tissues in specific organs. As a result, delivery vectors are chosen according to their attraction to certain cells and areas of the body.

One of the first delivery vectors used was retroviruses. Because these viruses are easily cultivated in a laboratory (artificially reproduced) scientists have studied them extensively and learned a great deal about their biological action. They also have learned how to remove, separate and modify the genetic information that governs viral replication, thus controlling the ability of viral replication and infection. Retroviruses work best in actively dividing cells, but many cells in the body are relatively stable after terminal differentiation and do not divide often, if at all. As a result, progenitors of these mature cells are used primarily for ex vivo (outside the body) manipulation. First, the cells are removed from the patient's body, and the virus, or plasmid vector, carrying the gene is infected, microinjected, or transfected. Next, the cells are cultivated in a nutrient-rich culture where they grow and replicate. Once enough cells are gathered, they are returned to the body, usually by injection into the blood stream. Theoretically, as long as these cells survive and reach the correct location, they will provide the desired therapy.

Another class of viruses, called the adenoviruses (cold viruses), also may prove to be good delivery vectors. These viruses can effectively infect non-dividing cells in the body expressing the Coxsackie and Adenovirus Receptor (CAR), where the desired gene product then is expressed naturally. These viruses live for several days in the body, and some concern surrounds the possibility of infecting others with the viruses through sneezing or coughing. Other viral vectors include Influenza viruses, Sindbis virus, and a Herpes virus that infects nerve cells.

Scientists also have delved into non-viral delivery gene delivery. This strategy relies on the natural biological process by which cells uptake (or gather) macromolecules. One approach is to use liposomes, globules of synthetic lipids or natural fat produced by the body and taken up by cells. Scientists also are investigating the introduction of raw recombinant DNA by injecting it into the bloodstream or placing it on microscopic beads of gold shot into the skin with a biolistic particle gun “gene-gun.” Another possible delivery vector under development is based on dendrimer molecules. A class of polymers (naturally occurring or artificial substances that have a high molecular weight and formed by smaller molecules of the same or similar substances), is “constructed” in the laboratory by combining these smaller monomer molecules. They have been used in manufacturing Styrofoam, polyethylene cartons, and Plexiglass. In the laboratory, dendrimers have shown the ability to transport genetic material into human cells. They also can be designed to form an affinity for particular cell membranes by attaching to certain sugars and protein groups.

In the early 1970s, scientists proposed “gene surgery” for treating inherited diseases caused by faulty genes. The idea was to take out the disease-causing gene and surgically implant a gene that functioned properly. Although sound in theory, scientists, then and now, lack the biological knowledge or technical expertise needed to perform such a precise surgery in the human body.

However, in 1983, a group of scientists from Baylor College of Medicine in Houston, Tex., proposed that gene therapy could one day be a viable approach for treating Lesch-Nyhan disease, a rare neurological disorder. The scientists conducted experiments in which an enzyme-producing gene (which produces a specific type of protein) for correcting the disease was injected into a group of cells for replication. The scientists theorized the cells could then be injected into people with Lesch-Nyhan disease, thus correcting the genetic defect that caused the disease.

As the science of genetics advanced throughout the 1980s, gene therapy gained an established foothold in the minds of medical scientists as a promising approach to treatments for specific diseases. One of the major reasons for the growth of gene therapy was scientists' increasing ability to identify the specific genetic malfunctions that caused inherited diseases. Interest grew as further studies of DNA and chromosomes (where genes reside) showed that specific genetic abnormalities in one or more genes occurred in successive generations of certain family members who suffered from diseases like intestinal cancer, bipolar disorder, Alzheimer's disease, heart disease, diabetes, and many more. Although the genes may not be the only cause of the disease in all cases, they may make certain individuals more susceptible to developing the disease because of environmental influences, like smoking, pollution, and stress. In fact, some scientists theorize that all diseases may have a genetic component.

On Sep. 14, 1990, a four-year old girl suffering from a genetic disorder that prevented her body from producing a crucial enzyme became the first person to undergo gene therapy in the United States. Because her body could not produce adenosine deaminase (ADA), she had a weakened immune system, making her extremely susceptible to severe, life-threatening infections that are generally benign to a normal individual. W. French Anderson and colleagues at the National Institutes of Health's Clinical Center in Bethesda, Md., took white blood cells (which are crucial to proper immune system functioning) from the girl, inserted ADA producing genes into them, and then transfused the cells back into the patient. Although the young girl continued to show an increased ability to produce ADA, debate arose as to whether the improvement resulted from the gene therapy or from an additional drug treatment she received.

Nevertheless, a new era of gene therapy began as more and more scientists sought to conduct clinical trial (testing in humans) research in this area. In that same year, gene therapy was tested on patients suffering from melanoma (skin cancer). The goal was to help them produce antibodies (disease fighting substances in the immune system) to battle cancer. These experiments have spawned an ever-growing number of attempts at gene therapies designed to perform a variety of functions in the body. For example, a gene therapy for cystic fibrosis aims to supply a gene that alters lung cells, enabling them to produce a specific chloride channel protein to battle the disease. Another approach was used to treat brain cancer patients, in which the recombinant gene was designed to make the cancer cells more likely to respond to drug treatment. Another gene therapy approach was used to treat patients suffering from artery blockage, which can lead to strokes and induces angiogenesis (the growth of new blood vessels) near clogged arteries, thus restoring normal blood circulation.

Currently, there are a host of new gene therapy agents in clinical trials. In the United States, both nucleic acid based (in vivo) treatments and cell-based (ex vivo) treatments are being investigated. Nucleic acid based gene therapy uses delivery vectors (like viruses) to deliver modified genes to target cells. Cell-based gene therapy techniques remove cells from the patient in order to genetically alter them then reintroduce them to the patient's body. Presently, gene therapies for the following diseases are being developed: cystic fibrosis (using adenoviral vector), HIV infection (cell-based), malignant melanoma (cell-based), Duchenne muscular dystrophy (cell-based), hemophilia B (cell-based), kidney cancer (cell-based), Gaucher's Disease (retroviral vector), breast cancer (retroviral vector), and lung cancer (retroviral vector). When a cell or individual is treated using gene therapy and successful incorporation of engineered genes has occurred, the cell or individual is said to be transgenic.

The potential scope of gene therapy is enormous. More than 4,200 diseases have been identified as resulting directly from abnormal genes, and countless others that may be partially influenced by a person's genetic makeup. Initial research has concentrated on developing gene therapies for diseases whose genetic origins have been established and for other diseases that can be cured or improved by substances genes produce.

The following are examples of potential gene therapies. People suffering from cystic fibrosis lack a gene needed to produce a chloride channel protein. This protein regulates the flow of chloride into epithelial cells, (the cells that line the inner and outer skin layers) that cover the air passages of the nose and lungs. Without this regulation, patients with cystic fibrosis build up a thick mucus that makes them prone to lung infections. A gene therapy technique to correct this abnormality might employ an adenovirus to transfer a normal copy of what scientists call the cystic fibrosis transmembrane conductance regulator, or CTRF, gene. The gene is introduced into the patient by spraying it into the nose or lungs. However, the aberrant channel in the diseased patient does not fold properly and precipitates inside the epithelial cells. A more ideal therapy would also remove the aberrant channel. Our invention also addresses this latter issue.

Researchers announced in 2004 that they had, for the first time, treated a dominant neurodegenerative disease called Spinocerebella ataxia type 1, with gene therapy. This could lead to treating similar diseases such as Huntington's disease. They also announced a single intravenous injection could deliver therapy to all muscles, perhaps providing hope to people with muscular dystrophy.

Familial hypercholesterolemia (FH) also is an inherited disease, resulting in the inability to process cholesterol properly, which leads to high levels of artery-clogging fat in the blood stream. Patients with FH often suffer heart attacks and strokes because of blocked arteries. A gene therapy approach used to battle FH is much more intricate than most gene therapies because it involves partial surgical removal of patients' livers (ex vivo transgene therapy). Corrected copies of a gene that serve to reduce cholesterol build-up are inserted into the liver sections, which then are transplanted back into the patients.

Gene therapy also has been tested on patients with AIDS. AIDS is caused by the human immunodeficiency virus (HIV), which weakens the body's immune system to the point that sufferers are unable to fight off diseases like pneumonias and cancer. In one approach, genes that produce specific HIV proteins have been altered to stimulate immune system functioning without causing the negative effects that a complete HIV molecule has on the immune system. These genes are then injected in the patient's blood stream. Another approach to treating AIDS is to insert, via white blood cells, genes that have been genetically engineered to produce a receptor that would attract HIV and reduce its chances of replicating. In 2004, researchers reported that had developed a new vaccine concept for HIV, but the details were still in development. Several cancers also have the potential to be treated with gene therapy. A therapy tested for melanoma, or skin cancer, involves introducing a gene with an anticancer protein called tumor necrosis factor (TNF) into test tube samples of the patient's own cancer cells, which are then reintroduced into the patient. In brain cancer, the approach is to insert a specific gene that increases the cancer cells' susceptibility to a common drug used in fighting the disease. In 2003, researchers reported that they had harnessed the cell killing properties of adenoviruses to treat prostate cancer. A 2004 report said that researchers had developed a new DNA vaccine that targeted the proteins expressed in cervical cancer cells.

Gaucher disease is an inherited disease caused by a mutant gene that inhibits the production of an enzyme called glucocerebrosidase. Patients with Gaucher disease have enlarged livers and spleens and eventually their bones deteriorate. Clinical gene therapy trials focus on inserting the gene for producing this enzyme.

Gene therapy seems elegantly simple in its concept: supply the human body with a gene that can correct a biological malfunction that causes a disease. However, there are many obstacles and some distinct questions concerning the viability of gene therapy. For example, viral vectors must be carefully controlled lest they infect the patient with a viral disease. Some vectors, like retroviruses, also can enter cells functioning properly and interfere with the natural biological processes, possibly leading to other diseases. Other viral vectors, like the adenoviruses, often are recognized and destroyed by the immune system so their therapeutic effects are short-lived. Maintaining gene expression so it performs its role properly after vector delivery is difficult. As a result, some therapies need to be repeated often to provide long-lasting benefits.

### DEFINITIONS

Cell—The basic structural and functional unit of all organisms.
Chromosome—A microscopic thread-like structure found within each cell of the body, consisting of a complex of proteins and DNA.
Clinical trial—The testing of a drug or some other type of therapy in a specific population of patients.
Organism Clone—A cell or organism derived through asexual (without sex) reproduction containing the identical genetic information of the parent cell or organism.
Deoxyribonucleic acid (DNA)—A form of genetic material consisting of a polymer of deoxyribose-phosphate scaffold and a specific sequence of adenine, cytosine, guanine, and thymine bases (the nucleobases) that holds the inherited instructions for growth, development, and cellular functioning.
Enzyme—A protein that catalyzes a biochemical reaction or change without changing its own structure or function.
Gene—A building block of inheritance, which contains the instructions for the production of a particular protein or RNA, and is made up of a molecular sequence found on a section of DNA. Each gene is found on a precise location on a chromosome.
Gene transcription—The process by which genetic information is copied from DNA to RNA.
Genetic engineering—The manipulation of genetic material to produce specific results in an organism.
Genetics—The study of hereditary traits passed on through the genes.
Genome—is the entirety of an organism's genetic material. It is encoded either in DNA or, for many types of virus, in RNA. The genome includes both the genes and the non-coding sequences of the DNA/RNA.
Germ-line gene therapy—The introduction of genes into reproductive cells or embryos to correct inherited genetic defects that can cause disease.
Liposome—Organization of lipids into a spherical bilayer.
Macromolecules—A large molecule composed of thousands of atoms.
Nitrogen—A gaseous element that is one type of atom in the base pairs in DNA.
Nucleus—The compartment in a eukaryotic cell that contains most of the cells genetic material, including chromosomes and DNA.
Protein—A polymer of amino acids which is an important building block of the body involved in the formation of body structures and controlling the basic functions of the human body.
Somatic gene therapy—The introduction of genes into tissue or cells to treat a genetic related disease in an individual.
TALEN—Transcription Activator-Like Effector Nucleases (TALENs) are artificial restriction enzymes generated by fusing the TAL effector DNA binding domain to a DNA cleavage domain.
Delivery Vector—Something used to transport genetic information to a cell.
Plasmid Vector or Cloning Vector—An element that carries inserted DNA and replicates in cells.
Expression Vector—A specialized type of plasmid that encodes the synthesis of a desired RNA in specific cell types.

### PRIOR ART

U.S. Pat. No. 7,785,792 (Wolffe) describes methods and compositions for targeted modification of chromatin structure, within a region of interest in cellular chromatin. Such methods and compositions are useful for facilitating processes such as, for example, transcription and recombination that require access of exogenous molecules to chromosomal DNA sequences.

Published U.S. Patent Application Document No. 2011/0145940 (Voytas et al.) discloses a method for modifying the genetic material of a cell, including: (a) providing a cell containing a target DNA sequence; and (b) introducing a transcription activator-like (TAL) effector-DNA modifying enzyme into the cell, the TAL effector-DNA modifying enzyme comprising: (i) a DNA modifying enzyme domain that can modify double stranded DNA, and (ii) a TAL effector domain having a plurality of TAL effector repeat sequences that, in combination, bind to a specific nucleotide sequence in the target DNA sequence, such that the TAL effector-DNA modifying enzyme modifies the target DNA within or adjacent to the specific nucleotide sequence in the cell or progeny thereof. The method may further provide to the cell a nucleic acid comprising a sequence homologous to at least a portion of the target DNA sequence, such that homologous recombination occurs between the target DNA sequence and the nucleic acid. The Voytas et al. application also describes a TALEN having an endonuclease domain and a TAL effector DNA binding domain specific for a target DNA, wherein the DNA binding domain having a plurality of DNA binding repeats, each repeat having a RVD that determines recognition of a base pair in the target DNA, wherein each DNA binding repeat is responsible for recognizing one base pair in the target DNA, and wherein the TALEN has one or more of the following RVDs: HD for recognizing C; NG for recognizing T; NI for recognizing A; NN for recognizing G or A; NS for recognizing A or C or G or T; N* for recognizing C or T; HG for recognizing T; H* for recognizing T; IG for recognizing T; NK for recognizing G; HA for recognizing C; ND for recognizing C; HI for recognizing C; HN for recognizing G; NA for recognizing G; SN for recognizing G or A; and YG for recognizing T.

TALEs were first discovered in the plant pathogen, Xanthomonas. TALEs bind to a specific DNA sequence and regulate plant genes during infection by the pathogen.

Each TALE contains a central repetitive region consisting of varying numbers of repeat units of typically 33-35 amino acids. It is this repeat domain that is responsible for specific DNA sequence recognition. Each repeat is almost identical with the exception of two variable amino acids termed the repeat-variable di-residues. The mechanism of DNA recognition is based on a code where one nucleotide of the DNA target site is recognized by the repeat-variable di-residues of one repeat.

A TALEN is composed of a TALE for sequence-specific recognition fused to the catalytic domain of an endonuclease that introduces double strand breaks (DSB). The DNA binding domain of a TALEN is capable of targeting with high precision a large recognition site (for instance 17 bp).

FIG. 2 is a schematic representation of the Structure and DNA-binding specificity of TALE proteins.

(a) Sketch of a TALE from Xanthomonas. Red rectangles indicate the central array of tandem repeats that mediate DNA recognition. A typical repeat sequence is provided above, with a box highlighting the RVD (positions 12 and 13) that determines base preference. Gray regions indicate flanking protein segments, which often contain 288 and 278 residues (left and right segments, respectively). Δ152 indicates a truncation point that disrupts TALE transport into plant cells but preserves other functions and which was used as the N terminus for all constructs in these studies. N and C denote N and C termini. (b) Base sequence preferences of four common RVDs^{23, 24}, which have been used in recent studies to make TALEs with new specificities (c) RVDs (top row of letters) and predicted target bases (second row of letters) for the natural protein TALE 13. RVDs are listed in repeat order (1 through 13), whereas the predicted target site is provided with the 5′ on the left. * denotes repeats that contain 33 amino acids, instead of the more typical 34. (d) Graphical depiction of a SELEX-derived base frequency matrix for a fragment of TALE 13 containing the repeat region.

Testing of TALENS is well reported in A TALE nuclease architecture for efficient genome editing. Jeffrey C. Miller et al., Nature Biotechnology 29, 143-148, (2011). Received 15 Nov. 2010, Accepted 14 Dec. 2010 and published online 22 Dec. 2010. Disclosed are nucleases that cleave unique genomic sequences in living cells can be used for targeted gene editing and mutagenesis. A strategy is developed for generating such reagents based on transcription activator-like effector (TALE) proteins from Xanthomonas. Identified are TALE truncation variants that efficiently cleave DNA when linked to the catalytic domain of the FokI nuclease and use of these nucleases to generate discrete edits or small deletions within endogenous human NTF3 and CCR5 genes at efficiencies of up to 25%. It is shown that designed TALEs can regulate endogenous mammalian genes. These studies demonstrate the effective application of designed TALE transcription factors and nucleases for the targeted regulation and modification of endogenous genes.

### SUMMARY OF THE INVENTION

Currently, large genomic segments can be deleted to generate knockout animals in model systems. Also Gene Therapy can be used to introduce copies of recombinant genes into people to replace missing activities. A major advance in application of basic science would be able to delete genomic fragments in patients. Our invention is a development in a technology to delete large regions of genomic DNA in people, animals or bacteria. The invention uses one or more P2E2 (Paired Permeant Endonuclease Excision) constructs consisting of a cell permeation component, a sequence specific DNA binding component, and an endonuclease component. Specificity is partially achieved though the DNA binding component, endonuclease cleavage site, and a requirement for tandemly opposed dimers (in tandem, at opposed positions on the chromosome strands) to cleave double stranded DNA. By using two sets of these cell permeant TALENs, one can target any region of any DNA-based genome for deletion, within some size limitations. A second part of this invention is for removal of viral genomes that are in an active or latent infection stage, as applied to HIV herein. The HIV P2E2 constructs target a repeated highly conserved TAR region site located near each termini of the HIV genome. Since the TALEN is attached to a cell permeant protein, it can be delivered, in this case by just injection of the purified P2E2 protein or by other delivery vectors such as recombinant viruses.

There is no current way to treat humans to delete pieces of DNA unless cells are removed from the body, manipulated, and implanted back in the body. Also in gene therapy there is no way to remove bad copies of genes. Our technology fills overcomes these limitations. Our technology also provides a mean for excising the HIV genome from infected humans. This can help to reduce or eliminate HIV infection including latency. There is currently no approach to remove latent viral sequences from genomes of patients. This technology can also be applied for treatment of many diseases, both of infectious and noninfectious nature.

### The P2E2 Construct

A P2E2 construct novel within the scope of the present invention can be generally described as a chemical tool for genome surgery comprising a P2E2 construct of, in the preferred order of, A) a cell penetration component, B) a DNA binding component and C) a restriction endonuclease. There are fundamentally only three possible orders, ABC, BAC and BCA, as any other combinations are merely reversals or functionally non-differentiable mirror images of the linear order of components (e.g., ABC=CBA). The DNA binding component and restriction endonuclease may be formed or commercially available according to the TAL, TALE or TALEN technology known in the art and described herein. The cell-penetration component is preferably affixed to the DNA binding component of the two-part DNA Binder and restriction endonuclease, but may also be attached to the restriction endonuclease end. It is possible to have the cell penetration component between the two other named segments, but its steric and physical location is likely to reduce its efficacy with regard to cell penetration and make alignment of the DNA binder and restriction endonuclease less precise.

P2E2 (Paired Permeant Endonuclease Excision) constructs for genome surgery and it methods of use in genome surgery are provided. A method for performing genome surgery may include:
a) providing one or more recombinant P2E2 constructs comprising, in order, a cell penetration component, a DNA binding component and an endonuclease;
b) penetrating a cell with the P2E2 constructs;
c) forming a protein product by the cellular processes of transcription and translation;
d) attaching the protein product of the P2E2 constructs to one ore more targeted genomic sequences within the cell; and
e) the endonuclease of the P2E2 construct cutting both strands of the genome at specific locations.

### BRIEF DESCRIPTION OF THE FIGURES

FIG. 1 is a schematic representation of a TALEN and its functionality.

FIG. 2 is a schematic representation of the structure and DNA-binding specificity of TALE proteins.

FIG. 3 is a schematically represented mediated transfection.

FIG. 4 is a schematic representation of transfection mediated by the formation of inverted micelles.

FIG. 5 is a schematic representation of transfection mediated by a transitory structure.

FIG. 6 shows a schematic representation of an example of transfection of cargo through direct penetration.

FIGS. 7A and 7B show an illustration that Restriction site (RES)#1 and #5 that are initially designated in the G-block design but once the CPP-endonuclease DNA is built, can be changed using forward (RES #1) and reverse (RES #5) primers combined with PCR for subcloning into a variety of plasmid vector backbones using different restriction endonucleases. (A) Basic construct structure before DNA binding domain (Tale) sub-cloning. (B) Basic construct structure after DNA binding domain (Tale) sub-cloning.

FIG. 8 shows a schematic of a process for synthesizing P2E2 constructs according to one aspect of the present technology.

FIG. 9 (A, B) show schematic formulae for Construct A DNA as to be double-digested with SalI and NotI to be eventually ligated into pGEX6P2 for bacterial expression of the protein for the P2E2 construct. Construct B DNA of FIG. 9 will be double-digested with NheI and EcoRV to be eventually ligated into pcDNA3.1(−)myc/his A for expression of the construct in eukaryotic cells.

FIG. 10 shows a schematic of an actual assembly sequence of steps used in forming P2E2 constructs.

FIG. 11 shows a vector and a blueprint for protein pairs of 5′Tal-FokI and 3′Tal-FokI DNA constructs.

FIG. 12 shows a spread on DNA-agarose gel visualizing DNA from an example based on size.

FIG. 13 shows stain evidencing that DNA constructs were functional blue prints that can be used by cellular machinery to produce RNA in a test tube, was designed to confirm the functionality of the synthesized protein pair.

### DETAILED DESCRIPTION OF THE INVENTION

The present invention includes various perspectives including at least a method for performing genome surgery including:
a) providing one or more recombinant P2E2 constructs comprising, in an ordered sequence, the preferred order being a cell penetration component, a DNA binding component and an endonuclease;
b) penetrating a cell with the recombinant P2E2 constructs or proteins;
c) forming a protein product in the cell by the processes of transcription and translation or by direct introduction to the cell;
d) attaching the protein product of the P2E2 constructs to one ore more targeted genomic sequences within the cell; and
e) the endonucleases of the P2E2 constructs cutting both strands of the genome at specific locations.

An alternative description of aspects of the invention may include a method for performing genome surgery including:
a) providing P2E2 constructs comprising, in order, a cell penetration component, a DNA binding component and an endonuclease;
b) penetrating a cell with recombinant P2E2 constructs or proteins;
c) attaching individual P2E2 constructs to two strands of a genome within the cell, the attaching of two individual P2E2 constructs positioning the endonuclease of each construct over a pair of sequences opposed to each other across a gap between strands; and
d) the endonuclease of each PSE2 construct cutting a strand of the genome at respective ones of the pair of sequences.

An alternative description of aspects of the invention may include a method for performing genome surgery on an integrated viral genome including:
a) identifying an integrated viral genome integrated within a host genome;
b) identifying one or more target regions of nucleic acid sequences within the integrated viral or bacterial genome;
c) providing one or more P2E2 constructs comprising, in order, a cell penetration component, a DNA binding component and a nuclease;
d) penetrating a cell with the recombinant P2E2 constructs or proteins;
e) attaching the P2E2 construct to a genome consisting of a viral integrated genome within a host genome within the cell;
f) the endonuclease of the P2E2 construct overlaying a section of the integrated viral genome; and
g) cutting both strands of the integrated viral genome.

Yet another alternative description of aspects of the invention may include a method for performing genome surgery on a bacterial genome including:
a) identifying a bacterial genome from a bacteria infecting a host;
b) identifying a target region of nucleic acid sequences within the bacterial genome;
c) providing P2E2 constructs comprising, in order, a cell penetration component, a DNA binding component and an endonuclease;
d) penetrating a cell with the recombinant P2E2 constructs or proteins;
e) attaching the P2E2 constructs to a bacterial genome of a bacteria infecting the host cell;
f) the endonucleases of the P2E2 constructs overlaying a section of the bacterial genome; and
g) cutting both strands of the bacterial genome in one or more regions.

In performing this technology, the following steps and materials are contemplated and enabled. In the method, the integrated or targeted or defective (e.g., viral) genome has two ends through which the integrated genome (e.g., an integrated viral genome) is attached within the host genome. Two pair of P2E2 constructs attach at each of the two ends of the integrated genome so that the endonuclease of each of the constructs overlays a section of the integrated genome. Two strands between each of the two ends of the integrated genome are cut, forming a segment of the previously integrated genome that is not attached to any portion of the host genome. The strands previously attached at the two free ends from which the segment was cut typically reattach without including the unattached segment there between. The reattachment of the ends need not be exact with insertions or deletions of up to ˜30 nucleotides. It is within the scope of the present practice to use (at least or exactly) two distinct and different pairs of P2E2 constructs in steps a), b), c), d), e) and f), and then in step g) a total of 4 DNA strand cuts are made, with two cuts each by each pair of P2E2 constructs. The genome segment may comprise an HIV genome segment, a Hepatitis [A, B or C] segment, or any other targeted genome segment as described by the approach herein. In some instances, as where there is some symmetry in the nature and types of available target sequences at various portions of the target, defective or integrated genome, only a single P2E2 construct may need to be used to make four cuts on the HIV genome segment. In other structures, or to distribute cuts at different locations, it is possible that only at least two pairs of P2E2 constructs are used to make four cuts on the HIV genome segment.

Another aspect of the present technology is a chemical tool for genome surgery comprising P2E2 constructs containing a cell penetration component, a DNA binding component and a restriction endonuclease. The three subunits may be in that order or may be rearranged.

An alternative description of aspects of the invention may include a method for performing genome surgery to remove an endogenous gene from an organism:
a) identifying a gene within an organism to be disrupted or deleted;
b) identifying one or more target regions of nucleic acid sequences within the organisms genome;
c) providing one or more P2E2 constructs comprising, in order, a cell penetration component, a DNA binding component and an endonuclease;
d) penetrating a cell with the recombinant P2E2 constructs or proteins;
e) attaching the P2E2 constructs to one or more specific regions of the genome within the cell;
f) the endonuclease of the P2E2 construct overlaying one or more sections of the target gene to be disrupted or removed; and
g) cutting both strands of the gene at one or more sites.

P2E2 constructs according to the present technology may be composed of at least three parts, which include the following: a cell penetrating peptide, a DNA binding domain, and an endonuclease. The cell penetrating peptide and the endonuclease can be constructed using a technique called Gibson Assembly to ligate the DNA pieces together, PCR to sew pieces of DNA together, can be obtained from existing plasmids, or generated by chemical synthesis. The DNA binding domain can be constructed using the Real Assembly kit (Addgene) or Golden Gate Assembly (Addgene). Once these DNA pieces are built/obtained, they can be inserted into mammalian and/or bacterial expression vectors using various methods including ligation dependent or independent cloning. The recombinant plasmid vectors will allow for the protein expression of the P2E2 constructs in either mammalian, insect, yeast, bacteria, or other cells. The resulting protein produced will consist of the cell penetrating peptide fused to a DNA binding domain fused to an endonuclease.

This technology is distinctly and functionally different from present forms of gene therapy. Even though the common definition of gene therapy would linguistically be generic to every possibly gene manipulation, including genome surgery, the actual techniques presently known and practiced are not the claimed technology of the present disclosure. Gene therapy is generally defined as something akin to the replacement or alteration of defective genes in order to prevent the occurrence of such inherited diseases as hemophilia. Gene therapy is usually affected by genetic engineering techniques. Gene therapy involves inserting copies of a normal allele into the chromosomes of an individual who carries a faulty allele. It is not always successful, and research is continuing.

The basic process of Gene therapy generally involves the following types of steps:
1. Doing research to find the gene involved in the genetic disorder.
2. Making many copies of the normal allele.
3. Putting copies of a gene with the normal allele into the cells of a person who has the genetic disorder. This may alternatively be performed by combining deletion of the gene containing the bad allele with P2E2 constructs and gene replacement with a gene containing the normal replacement allele by standard gene therapy approaches.
4. Reintroducing correct cell copies into the patient.

These steps are often performed ex vivo with the “corrected” cells then reintroduced into the body by injection, infusion or perfusion. The present genome surgery removes any identified defective sequences in the genome and then reattaching the cut ends of the underlying patient genome so that a significant (and assumed adverse) functionality of the defective sequences may be also moderated. Appreciation of this difference is significant. According to the present technology, this method may be done by injection, perfusion, diffusion or infusion of the novel proteins of the present technology into the host.

Our approach enabling genome surgery should be generically considered in the following manner. Healthy or correct patient genomes in a single strand shall be considered, for purposes of illustration only, to be represented by the following allegorical representation:

GCATGGCCAATTGCATAACCGGTTGGCCAATTGCATGGCCAATT

A specific defect in genome structure shall be allegorically referred to as

WWXXYYZZ-ZZYYXXWW

The defective genome structure would therefore be allegorically represented as:

GCATGGCCAATTGCAT-WWXXYYZZ-ZZYYXXWW-
AACCGGTTGGCCAATTGCATGGCCAATT

The adverse function of the defect (e.g., a latent virus or other defective sequence) within the genome is usually a contribution of the collective activity of the defective sequence within the genome or a gene in the genome with a alelle that impairs the genes function. Removing the adverse affects does not necessarily (and seldom does) require removal of every single nucleic acid within the defective sequence “WWXXYYZZ-ZZYYXXWW,” but rather removal of only a section of the defective genome (e.g., WWXXYYZZ-ZZYYX; XYYZZ-ZZYYXXWW; WWXXXWW; etc.) is usually sufficient to inactivate the harmful activity of the defective genetic sequence. This sequence excising, whether complete, partial, symmetrical, asymmetrical or the like, is usually, if not always sufficient to eliminate the adverse effects of the genetically undesirable sequence within the genome. The most easily understood example of this is where the defect is an embedded or latent viral genome. If a significant (e.g., as few as 1 nucleic acids within a single strand) sequence length is removed, the virus genome can become effectively deactivated. It is preferred that at least 10%, at least 20%, at least 30%, at least 40%, at least 50%, at least 60%, at least 70%, at least 80%, at least 90% or at least 95% of the defective genetic sequence is removed.

As a corrolary, it is desirable that the underlying host genome is not disrupted, or significant segments of the host genome are not removed by the genome surgery in which a portion of the defective genome sequence is removed. For example, the following residues in genetic surgery resulting from the allegoric genome sequence with errors of:

GCATGGCCAATTGCAT-WWXXYYZZ-ZZYYXXWW-
AACCGGTTGGCCAATTGCATGGCCAATT

Could allegorically include at least:

```
a) GCATGGCCAATTGCAT-WW-ZZYYXXWW-
      AACCGGTTGGCCAATTGCATGGCCAATT
b) GCATGGCCAATTGCAT-WWXXXWW-AA
      CCGGTTGGCCAATTGCATGGCCAATT
c) GCATGGCCAATTGCAT-WWXXYYXWW-A
      ACCGGTTGGCCAATTGCATGGCCAATT
d) GCATGGCCAATTGCAT-WWXXYYYXXWW-
      AACCGGTTGGCCAATTGCATGGCCAATT
```

At the same time, it would be less preferred if not undesirable to reduce the nucleic acids or nucleobases in the underlying host genome as in the following less preferred examples:

```
e) GCATGGCCAATTGCZZ-ZZYYXXWW-A
      ACCGGTTGGCCAATTGCATGGCCAATT
f) GCATGGCCAATTGCAT-WWXXYYZZ-
      ZGTTGGCCAATTGCATGGCCAATT
```

The targeting of the sequences to be removed requires both a chemical positioning and geometric positioning of the restricting enzyme at the cut site in the genome sequence upon which surgery is to be performed. That is, the chemical makeup of the construct must attach at a specific location and the geometric and length of the connecting elements and the restrictive enzyme in the construct must position the active portion of the enzyme at the specific sequence that is to be cut. The underlying procedure for alignment is understood from the existing work on TALE technology, TALEN and TALENS, and the present technology advances that background in at least two different ways:
1) The cell penetration functionality is present within the P2E2 construct; and
2) The present technology process cuts the target or defective sequence at two sites within the target sequence and excises a sufficient portion of the genome sequence as to deactivate the activity encoded by the sequence.
3) The endonuclease component can be for a specific cleavage site generating higher specificity for cleaving the genome, rather than the use of nonspecific FOKI endonuclease in the TALEN technology
4) In the case of HIV, the cell permeant component, the Tat protein, can also serve to pass between cells and reactivate latent HIV virus production.

In the TALEN technology, a single cut is made in the genome sequence (although it cuts both strands of the DNA within a small range of nucleotides), and then the process allows for normal biological functions of the body to correct, repair, alter, reconstruct and recombine the cut sequences into a new order in which the target or defective sequence may become deactivated. One skilled in the art may also used pairs of TALENs to cut at more than one site to remove larger pieces of the genome sequence.

An example of a P2E2 construct that has the cell penetrating (CP) component, binding component (BC) and restricting enzyme (RE) components and how they would align with a defective enzyme is shown below in again allegorical format:

CP---------BC---------------RE RE-------------------BC-----CP
GCATGGCCAATTGCAT-WWXXYYZZ-ZZYYXXWW-
AACCGGTTGGCCAATTGCATGGCCAATT

As can be seen from the alignment of elements, the binding component is positioned in relationship to the TTG sequence (positioning the construct) and the restriction enzyme is positioned over the XXY sequence, which is to be cut. Note that if the BC were attached to a different TTG sequence in the genome sequence, there would be no alignment of the RE with a XXY sequence. As the enzyme is sequence specific, the RE would not make a cut elsewhere in the genome sequence.

The invention also may include a chemical tool for genome surgery, which includes P2E2 constructs of in order, a cell penetration component, a DNA binding component and a restriction endonuclease. The details for each component are provided in the following three sections

Cell Permeation components

The cell-penetrating or cell-penetration component or segment may be a chemical or a virus, bacteria or preferably a peptide, such as a TAT peptide, or the cell-permanent piece of the tat protein. Cell-penetrating peptides (CPPs) are of different sizes, amino acid sequences, and charges but all CPPs have one distinct characteristic, which is the ability to translocate proteins across the plasma membrane and facilitate the delivery of various molecular cargoes to the cytoplasm or an organelle. There has been no real consensus as to the mechanism of CPP translocation, but the theories of CPP translocation can be classified into three main entry mechanisms: direct penetration through the membrane, endocytosis-mediated entry, and translocation through the formation of a transitory structure. CPP transduction is an area of ongoing research.

Cell-penetrating peptides (CPP) are able to transport different types of cargo molecules across plasma membrane; thus, they act as molecular delivery vehicles which can be used for delivery in live organisms. Cell-penetrating peptides have found numerous applications in medicine as drug delivery agents in the treatment of different diseases including cancer and virus inhibitors, as well as contrast agents for cell labeling. Examples of the latter include acting as a carrier for GFP, MRI contrast agents, or quantum dots.

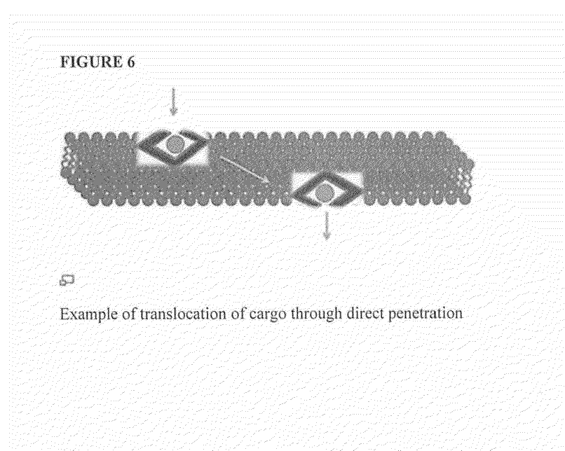

Example of translocation of cargo through direct penetration is schematically represented by FIG. 6.

The majority of early research suggested that the translocation of polycationic CPPs across biological membranes occurred via an energy-independent cellular process. It was believed that translocation could progress at 4° C. and most likely involved a direct electrostatic interaction with negatively charged phospholipids. Researchers proposed several models in attempts to elucidate the biophysical mechanism of this energy-independent process. Although CPPs promote direct effects on the biophysical properties of pure membrane systems, the identification of fixation artifacts when using fluorescent labeled probe CPPs caused a reevaluation of CPP-import mechanisms. These studies promoted endocytosis as the translocation pathway. An example of direct penetration has been proposed for Tat, a protein made by HIV. The first step in this proposed model is an interaction with the unfolded fusion protein (Tat) and the membrane through electrostatic interactions, which disrupt the membrane enough to allow the fusion protein to cross the membrane. After internalization, the fusion protein refolds due the chaperone system. This mechanism was not agreed upon, and other mechanisms involving clathrin-dependent endocytosis have been suggested.

Recently, a detailed model for direct translocation across the plasma membrane has been proposed. This mechanism involves strong interactions between cell-penetrating peptides and the phosphate groups on both sides of the lipid bilayer, the insertion of charged side-chains that nucleate the formation of a transient pore, followed by the translocation of cell-penetrating peptides by diffusing on the pore surface. This mechanism explains how key components or ingredients, such as the cooperativity among the peptides, the large positive charge, and specifically the guanidinium groups or arginine residues, contribute to the uptake. The proposed mechanism also illustrates the importance of membrane fluctuations. Indeed, mechanisms that involve large fluctuations of the membrane structure, such as transient pores and the insertion of charged amino acid side-chains, may be common and perhaps central to the functions of many membrane protein functions. This model contains several controversial features, maybe the most striking one is the formation of transient pores that facilitate the diffusion of the peptides across either the plasma membrane or the endosomal vesicles towards the cytosol. Recent experimental data has validated this key ingredient or components of the model showing that cell-penetrating peptides indeed form transient pores on lipid bilayers and on live cells.

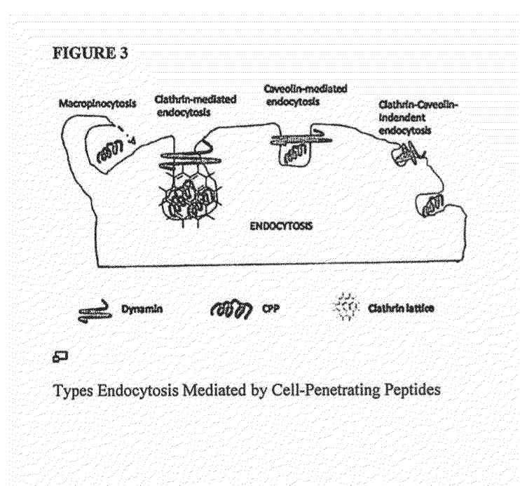

Endocytosis mediated Translocation is schematically represented in FIG. 3.

Endocytosis is the second mechanism liable for cellular internalization. Endocytosis is one type of process of cellular ingestion by which the plasma membrane folds inward to bring substances into the cell. During this process cells absorb material from the outside of the cell by imbibing it within vescile formed from their plasma membrane. The classification of cellular localization using fluorescence or by endocytosis inhibitors is the basis of most examination. However, the procedure used during preparation of these samples creates questionable information regarding endocytosis. Moreover, studies show that cellular entry of the Penetratin CPP by endocytosis is an energy-dependent process. This process is initiated by polyarginines interacting with heperan sulphates that promote endocytosis. Research has shown that Tat is internalized through a different type of endocytosis called macropinocytosis.

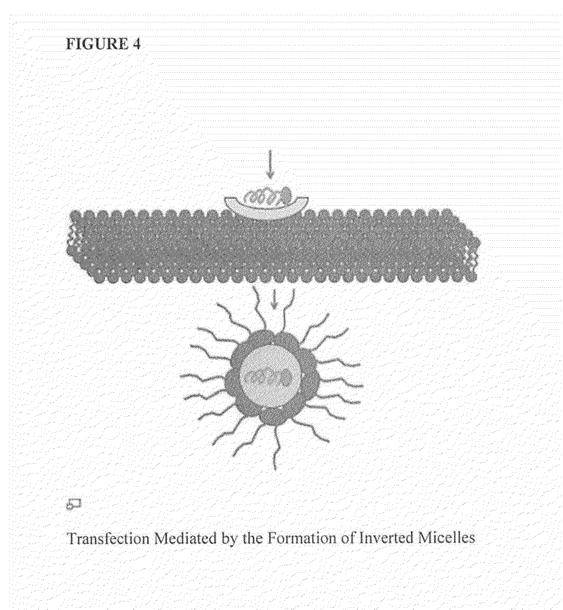

Studies have illustrated that endocytosis is involved in the internalization of CPPs, but it has been suggested that different mechanisms could transpire at the same time. This is established by the behavior reported for Penetratin and Transportan CPPs, wherein both membrane translocation and endocytosis occur concurrently. Translocation Mediated by the Formation of Inverted Micelles is schematically represented in FIG. 4.

The third mechanism responsible for the translocation is based on the formation of the inverted micelles. Inverted micelles are aggregates of colloidal surfactants in which the polar groups are concentrated in the interior and the lipophilic groups extend outward into the solvent. According to this model, a Penetratin dimer combines with the negatively charged phospholipids, thus generating the formation of an inverted micelle inside of the lipid bilayer. The structure of the inverted micelles permits the peptide to remain in a hydrophilic environment. Nonetheless, this mechanism is still a matter of discussion, because the distribution of the Penetratin between the inner and outer membrane is asymmetric. This asymmetric distribution produces an electrical field that has been well established. Increasing the amount of peptide on the outer leaflets causes the electric field to reach a critical value that can generate an electroporation-like event.

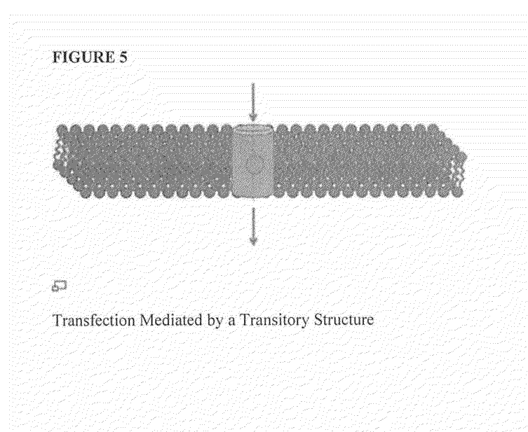

The last mechanism implied that internalization occurs by peptides that belong to the family of primary amphipathic peptides, MPG and Pep-1. Two very similar models have been proposed based on physicochemical studies, consisting of circular dichroism, Fourier transform infrared, and nuclear magnetic resonance spectroscopy. These models are associated with electrophysiological measurements and investigations that have the ability to mimic model membranes such as a monolayer at the air-water interface. The structure giving rise to the pores is the major difference between the proposed MPG and Pep-1 model. In the MPG model, the pore is formed by a β-barrel structure, whereas the Pep-1 is associated with helices. In addition, strong hydrophobic phospholipid-peptide interactions have been discovered in both models. In the two peptide models, the folded parts of the carrier molecule correlate to the hydrophobic domain, although the rest of the molecule remains unstructured. Translocation mediated by a transitory structure is schematically represented by FIG. 5.

CPP facilitated translocation is a topic of great debate. Evidence has been presented that translocation could use several different pathways for uptake. In addition, the mechanism of translocation can be dependent on whether the peptide is free or attached to cargo. The quantitative uptake of free or CPP connected cargo can differ greatly but studies have not proven whether this change is a result of translocation efficiency or the difference in translocation pathway. It is probable that the results indicate that several CPP mechanisms are in competition and that several pathways contribute to CPP internalization.

During the last decade, an important, new approach to the intracellular delivery of macromolecules and nanocarriers has emerged. This is based on ‘protein-transduction domains’ (PTDs) also known as Cell Penetrating Peptides (CPPs). The prototypical CPPs are short cationic peptides (Tat, ANT) derived from the transcriptional regulator proteins HIV Tat and drosophila Antennepedia; ‘Tat’ and ‘ANT’ have now been joined by a large number of additional CPPs. Many CPPs have a polycationic character, but others are based on hydrophobic sequences derived from signal peptides, viral peptides, or other sources. CPPs can not only enter cells themselves but, with greater or lesser efficiency, can also transport attached ‘cargo’ molecules. However, the efficiency of delivery is affected by the nature of the cargo. Certain CPPs have very effectively deliver biologically active (but normally membrane impermeant) short peptides, thereby allowing some role of these active peptides in signaling processes. Cationic and hydrophobic CPPs have also been reported to permit intracellular delivery of proteins into cultured cells, as well as in vivo delivery of enzymes such as f-galactosidase and Cre recombinase to cells in tissues. Tat and ANT variety of CPPs have also been used for the intracellular delivery of antisense and siRNA oligonucleotides. Even the delivery of large entities such as liposomes and magnetic nanoparticles can be enhanced via CPPs. Although various CPPs can cause cytotoxicity when used at high levels, for the most part they are relatively nontoxic when used at low concentrations.

More recent live-cell studies indicate that most cationic CPPs enter cells by binding to cell-surface proteoglycans, followed by uptake into endosomes most likely by macropinocytosis, followed by partial release from endosomes via a pH-dependent mechanism. As a result of this process, substantial amounts of these cationic peptides (and their cargos) remain within the endosomal compartment. It is expected that a CPP linked to a small peptide might undergo a different cell entry process than CPPs linked to a much larger nanocarrier. The mechanism(s) involved in the passage of CPPs and their cargos across endo-membranes are still poorly understood, but there are many known CPPs that are available for linkage to various cargos, including the remaining components of the constructs of the present technology.

Tables 1-5 below show examples of known CPPs and Cell Targeting Peptides (CTPs, for binding to specific molecules in cells reported in the literature and cargo combinations, evidencing the fact that these and other CPPs may be used in the practice of the present technology.

TABLE 1
Peptide-protein Conjugates

| CPP or | Nanocar- | Therapeutic or | In vivo or | |
|---|---|---|---|---|
| CTP | rier | imaging agent | in vitro | Purpose of study |
| RGD | PEG- | P38 MAPK | In vitro | Inhibition of |
| | albumin | inhibitor | | angiogenesis |
| RGD | PEG- | Auristatin | In vitro | Targeting of tumor |
| | albumin | (anti-cancer | | or endothelial |
| | | drug) | | cells |
| Arginine- | Albumin | NA | In vitro | Screen for |
| rich | | | | cell-penetrating |
| cyclic | | | | peptides |
| peptides | | | | |
| Arginine- | NA | Insulin | In vivo | Enteric delivery |
| rich | | | | of insulin |
| peptides | | | | |

TABLE 2
Peptide-nanoparticle Conjugates

| | | Therapeutic or | |
|---|---|---|---|
| CTP or CPP | Nanocarrier | imaging agent | Purpose of study |
| RGD | 3-Aminopropyltrimethoxysilane | Iron oxide | In vivo MRI |
| | (APTMS) | | |
| Thiolated | N-[{w-[4-(p-maleimidophenyl) | Gadolinium | In vivo MRI |
| peptidomimetic | butanoyl]amino} poly(ethylene | (Gd³⁺) | |
| vitronectin | glycol)₂₀₀₀] 1,2-distearoyl-sn- | | |
| antagonist | glycero-3-phosphoethanolamine | | |
| | (MPB-PEG-DSPE) | | |
| TAT | Aminated dextran | Iron oxide | In vivo MRI, cell tracking |
| TAT | Aminated dextran | Iron oxide, | In vivo |
| | | fluorochromes | imaging via |
| | | (VT-680, AF680, | fluorescence and MRI |
| | | Cy5 and Cy5.5) | |
| Transferrin | PGLA | Paclitaxel | In vivo tumor therapy |
| Transferrin | Cyclodextrin | siRNA | In vivo preclinical |
| | | | toxicology of siRNA in a |
| | | | nanoparticle |
| Deslorelin, | Polystyrene nanoparticle | | Ex vivo |
| transferrin | | | |
| Transferrin | Mercaptoundecanoic acid, lysine | CdSe/CdS/ZnS | In vitro imaging of |
| | | quantum rods | cancer cells |

TABLE 3
Peptide-polymer Conjugates

| | | Therapeutic or | |
|---|---|---|---|
| CPP or CTP | Nanocarrier | imaging agent | Purpose of Study |
| RGD | PEG-PEI | pCMV-sFit-1 | In vivo antiangiogenic |
| | | | tumor therapy |
| CD21 receptor biding | HPMA | N.A. | In vitro screening for |
| peptide (RMW- | | | targeting peptides |
| PSSTVNLSAGRR) | | | |
| Mono and doubly | HPMA | Indium-111 | In vivo targeting of avb3 |
| cyclized RGD | | | integrin in tumors |
| RGD | PEG-PEI, PEI | pGL3 plasmid | In vitro Gene delivery |
| | | | via integrins |
| Transferrin | PEG-PEI | pCMVL plasmid | In vivo gene delivery to |
| | | | Tumors |
| Tat | PEG-PEI | pGL3 plasmid | In vivo DNA delivery to |
| | | | Lung |
| CD-13 binding | PEG-PEI | β-Gal plasmid, | In vivo gene delivery to |
| peptide (CNGRC) | | YFP plasmid | tumors |
| Transferrin | PEG-PEI, PEI | CMV fl plasmid | In vivo fluorescence |
| | | | imaging of tissues |
| Tat | HPMA | Dox, FITC, Texas | In vitro uptake by tumor |
| | | Red | Cells |
| Tat, Lys9 | PEG | siRNA | In vitro siRNA uptake |
| Tat | PLLA-PEG, | Dox | In vitro delivery of |
| | Poly(methacryloyl sul- | | antitumor drugs to cells |
| | fadimethoxine)-PEG | | |
| | (PSD-PEG) | | |

RGD: Arg-Gly-Asp; PEG: Poly(ethyleneglycol); PEI: Polyethyleneimine; HPMA: Hydroxy Poly methacrylate.

TABLE 4
Peptide-liposome Conjugates

| | | Therapeutic or | |
|---|---|---|---|
| CPP or CTP | Nanocarrier | imaging agent | Purpose of study |
| RGD | Sterically stabilized | Doxorubicin (anticancer/ | Improve antitumor efficacy |
| | liposome | antiproliferative drug) | of doxorubicin (in vivo) |
| RGD | Sterically stabilized | Dexamethasone | Inhibition of angiogenesis |
| | liposome | phosphate | and thereby experimental |
| | | (anti-inflammatory) | arthritis (in vivo) |
| RGD | Liposomes | B¹⁰ (dodecahydrodode- | Inhibition of angiogenesis |
| | | caborate) | by targeting tumor |
| | | radiotherapeutic agent | vasculature (in vitro) |
| | | for neutron capture | |
| | | therapy | |
| RGD | Sterically stabilized | 5-Fluorouracil | Inhibition of lung |
| | liposome | (anticancer agent) | metastasis and |
| | | | angiogenesis in mice |
| Transferrin | Sterically stabilized | Citicoline | Drug targeting to brain by |
| | liposome | (neuroprotective agent) | targeting cells of the |
| | | | blood-brain barrier (in |
| | | | vitro) |
| Growth factor | Sterically stabilized | Doxorubicin (anti- | Targeting small-cell lung |
| antagonist [D-Arg⁶, D- | liposome | cancer/antiproliferative | carcinoma cells (in vitro) |
| Trp^{7,9}-N^{me}Phe⁸]- | | drug) | |
| substance P(6-11) | | | |
| antagonist G | | | |
| Epidermal growth factor | Sterically stabilized | B¹⁰ (boronated acridine) | Boron neutron capture |
| | liposome | radiotherapeutic agent | therapy for cancer cells (in |
| | | for neutron capture | vitro) |
| | | therapy | |
| TAT | pH-sensitive PEG-coated | Nontherapeutic plasmid | Development of |
| | liposomes | encoding green | tumor-specific |
| | | fluorescent protein | stimuli-sensitive drug and |
| | | | gene delivery (in vivo) |

TABLE 5
Direct Conjugates with Drugs and Imaging Agents

| | | Therapeutic or | |
|---|---|---|---|
| CPP or CTP | Nanocarrier | imaging agent | Purpose of study |
| RGD | PEG | Radiotracer | Tumor imaging and therapeutic |
| | | (⁶⁴Cu-DOTA-PEG-RGD). | applications. |
| RGD | N/A | Doxorubicin-RGD-4C, acts as | Tumor targeting. |
| | | tumor inhibitor. | |
| Dimeric cyclic | N/A | Radiolabeled-RGD peptide, a | To study specific tumor uptake |
| RGD | | potential imaging and | as well as therapy of |
| | | therapeutic agent. | radiolabeled dimeric RGD |
| | | | peptide. |
| Dimeric cyclic | N/A | Paclitaxel, an antimicrotubule | The potential of tumor- |
| RGD | | agent, a potent antitumor drug. | targeted delivery of |
| | | | paclitaxel-RGD conjugate and |
| | | | its utilization as antitumor |
| | | | agent. |
| Tetrameric cyclic | N/A | ⁶⁴Cu-DOTA-E{E[c(RGDfk)]₂}₂: | To investigate integrin |
| RGD | | microPET imaging of glioma | targeting characteristics of |
| | | integrin αᵥβ₃ expression. | ⁶⁴Cu-DOTA-E{E[c(RGDfk)]₂}₂ |
| | | | as a potential agent for |
| | | | diagnosis and |
| | | | receptor-mediated internal |
| | | | radiotherapy of integrin |
| | | | receptor-expressing tumors. |
| Multimeric RGD | N/A | Cypate, an optical imaging | Design, synthesis and |
| | | agent. | evaluation of multimeric arrays |
| | | | of RGD peptides on a |
| | | | near-infrared fluorescent dye |
| | | | (cypate) for tumor targeting. |
| RGD-tetramers | N/A | [^{99m}Tc(HYNIC-tetramer) | To explore the impact of |
| | | (tricine)(TPPTS)] is a new | peptide multiplicity on |
| | | promising radiotracer for | biodistribution characteristics |
| | | noninvasive imaging of the | and metabolism of the |
| | | integrin αᵥβ₃-positive tumors | ^{99m}Tc-labeled multimeric cyclic |
| | | by SPECT. | RGDfk peptides. |
| Cyclic RGD | N/A | [^{99m}Tc(CO)₃-cyclo- | Targeting integrin receptors |
| | | [RGDyk(PZ)]]⁺, a potential | upregulated on tumor cells and |
| | | imaging agent to visualize | neovasculature. |
| | | angiogenesis and tumor | |
| | | formation in vivo. | |
| Neurotensin | N/A | NT-XI, NT-XII, NT-XIII; new NT | Development of |
| (NT), a | | analogs for imaging tumors. | double-stabilized neurotensin |
| tridecapeptide | | | analogs as potential |
| | | | radiopharmaceuticals for the |
| | | | application in tumor imaging |
| | | | and potentially, therapy of NT |
| | | | receptor-positive tumors. |
| Bitistatin | N/A | Labeled bitistatin, a promising | Targeting αᵥβ₃ integrins in |
| (polypeptide) | | in vivo imaging agent. | tumor angiogenesis. |
| Transferrin | Transferrin | Transferrin (Tf), an anticancer | To enhance the intracellular |
| | receptor (TfR) | drug delivery agent. | drug release in cultured tumor |
| | | | cells by Tf-oligomers. |

RGD: Arg-Gly-Asp: PEG: Poly(ethyleneglycol); DOTA. 1,4,7,10-tetraazacyclododecane-N,N′,N′′,N″′-tetraacetic acid; HYNIC: 6-hydrazinonicotinamide; TPPTS: trisodium triphenylphosphine-3,3^{!},3^{!!!}trisulfonate; SPECT: single photon emission computed tomography.

Chemical transporters may be used in place of the cell transporting peptides. These also enhance the translocation of drugs or probes across biological barriers. The entry of these agents into cells is not a function of their peptide structure but rather, in the case of the arginine-rich agents, the number and spatial array of their guanidinium groups. Indeed, in a definitive series of structure-function studies starting in the 1990s and continuing to the present, include spaced peptide, peptoid, carbamate, carbonate and dendrimeric scaffolds readily enter cells provided that they are decorated with the appropriate number and arrangement of guanidinium groups.

The function of these Molecular Transporters (MoTrs), in this case translocation into a cell, can thus be mimicked and even improved upon with alternative simplified structures. It has been shown that guanidinium-rich (GR) dendrimers, beta-peptides, foldamers, carbohydrates, PNAs, morpholinos, bicyclic guanidiniums and other non-natural scaffolds can translocate into cells. GR-MoTrs have also been shown to cross other biological barriers including skin, blood-brain, ocular, buccal and membranes of intracellular organelles. Cargoes, which can be either noncovalently associated with or covalently attached to these MoTrs, include small molecules, imaging agents, metals, peptides, proteins, plasmids and siRNA. Transport of larger assemblies (e.g., quantum dots, iron particles, vesicles) has also been enhanced by guanidinylation. For cases in which free cargo is required to be released after cell entry, the linker through which the cargo is attached to the transporter can be cleaved by either a biological method, including light, pH and heat, or by biological activation including protease, esterase, phosphatase and redox reactions. Significantly, the transporter-cargo conjugate can be targeted to cells and tissue by ‘turning off’ the oligocation molecular transporter function through attachment to an oligoanion and then ‘turning on’ uptake by cleavage of the attached oligoanion using local cellular or tissue biochemistry.

Molecular transporter technology has progressed to clinical trials initially for the treatment of psoriasis using cyclosporin-heptaarginine conjugates and subsequently for the treatment of ischemic damage using RACK peptide-transporter conjugates. Significantly, GR-MoTr drug conjugates have also been shown to overcome multidrug-resistant cancer in cellular and animal models, even when the drug alone succumbs to resistance. Further therapeutic and research applications of MoTrs beyond small molecules can be expected as they provide a solution to the singularly most significant problem associated with the clinical use of biologics, namely delivery. GR-MoTrs can be used to effect uptake of a long list of probes, drugs and drug leads. Of particular interest to the theme of this publication, GR-MoTrs are effective for the delivery of peptides and proteins. Traditionally considered ‘undruggable’ due to their metabolic instability and general inability to cross biological membranes, many peptides and proteins can be delivered into cells with MoTr technology. Indeed, an impressive example of this capability was the early demonstration that an active beta-galactosidase protein could be delivered across the blood-brain barrier in mice by conjugation to the Tat peptide. More recently oligoarginine-protein fusion constructs have been used to deliver transcription factors to reprogram somatic cells to induced pluripotent stem cells. Among the first peptides delivered with oligoarginine transporters were the RACK octapeptide and Cyclosporin A. Both have progressed into clinical trials.

MoTrs can also be designed to target intracellular organelles such as the nucleus and mitochondria. Of particular importance with regard to clinical implementation is the ability to access these GR-MoTrs with cost-effective, step-economical synthetic strategies. In this regard, GR-homooligomers offer significant cost and scale advantages in addition to often better performance and tunability relative to the original Tat-9-mer peptide.

Nonpeptidic GR-MoTrs

### Linear GR-MoTrs

The first nonpeptidic GR-MoTrs were GR oligopeptoids. While retaining the same 1,4 side chain spacing of the peptide transporters and an amide bond, these peptoid transporters exhibited more flexibility both along the backbone and between the backbone and side chain. Significantly, they worked better than peptides in comparative uptake studies with Jurkat cells, showing clearly that a conventional peptidic amide bond is not required for cell entry. That more flexible systems would work better is consistent with the dynamics of cell entry rather than an affinity-based recognition process for which pre-organization would be important. Given that the backbone stereochemistry and substitution could be varied, research was next directed at the effect of backbone spacing and composition on uptake. It was found that introduction of aminocaproic acid spacers between arginine groups resulted in GR-MoTrs that outperformed oligomers of arginine alone. b-Peptides, which contain one additional methylene unit between guanidinium containing side chains, showed similar behavior to the a-peptide scaffold: the b-oligoarginine performed well, while the b-oligolysine was less effective. An additional and important question was whether the peptide or peptoid backbone could be more dramatically modified. Aminocaproic acid spacers between arginines may provide better cellular uptake.

In addition to linear scaffolds, dendrimeric and other branched GR-MoTrs have been shown to be effective in promoting cellular entry. The first branched scaffolds were based on an amino acid backbone with lysine residues as branch points. As had been shown for the linear systems, uptake was dependent on the guanidinium content (number of arginine residues). GR-MoTrs based on dendrimeric scaffolds have been reported. As with the linear scaffolds, uptake was found to be dependent on the number of guanidinium groups, with at least six being required for rapid uptake. Shorter oligomers undergo uptake which, while slow, could still be clinically relevant. In addition to the primary importance of the guanidinium groups, work on dendrimeric scaffolds has shown that the scaffold can also play a role in uptake efficiency. In this work different scaffolds, which had the same number of guanidinium groups but differed in spacing of these groups along the dendrimeric backbone, were analyzed for cellular uptake. Significantly, the most effective of these dendrimeric GR-MoTrs outperformed nonaarginine, while the least flexible dendrimers did not undergo rapid cellular uptake. Collectively, from a design perspective, these studies indicate that a range of scaffolds, if properly decorated with guanidinium groups, could be used to achieve cell entry.

### Other Scaffolds of GR-MoTrs (Guanidinylation of Cargo)

Because of the singular importance of the guanidinium group for cellular uptake and the flexibility that is allowable in the display of these guanidinium moieties, it follows that simply guanidinylating a cargo could be used to enhance its cellular uptake. For example, guanidinylation of oligonucleotides enhances cellular uptake relative to the parent unguanidinylated scaffold. Guanidinylation strategies for oligonucleotides have included peptide nucleic acids with insertion of arginine along the backbone, guanidinylation at the C5 site of a modified deoxyuridine, guanidinylation via attachment of an N-alkyl through the phosphate group of the phosphate backbone and the replacement of the phosphate group with guanidinium groups along the oligonucleotide backbone. All of these varied guanidinylation strategies resulted in systems exhibiting enhanced cellular uptake.

In addition, the guanidinylation of aminoglycosides, including tobramycin and neomycin B, has proven to be an effective strategy for the enhanced cellular uptake of these carbohydrates. The resulting guanidinoglycosides exhibited sustained or improved biological function relative to the unmodified scaffold, in one case showing 100-fold greater inhibition of HIV viral replication by guanidinotobramycin and guanidinoneomycin B. These guanidinoglycosides can also act as GR-MoTrs and have been shown to deliver large (>300 kDa) bioactive cargoes into cells.

Guanidinylated carbohydrate scaffolds based on inositol and sorbitol have also been shown to readily enter cells. The sheer variety of guanidinylation patterns and strategies and the range of cargoes that have been carried into cells via these strategies highlights the versatility and power of oligoguanidinylation for enabling or enhancing cellular uptake.

When delivering P2E2s as proteins it may be necessary to mask the protein from the immune systems. This can be by a process called PEGelation. Proteins can be PEGylated by any of a large number of available chemical groups that can be used to enable esterification reactions, etherification reactions, ethylenic reactions, addition reactions, condensation reactions, hydrolysis, inter-PEGelation, and the like.

The process may also be referred as “heterobifunctional” or “heterofunctional.” The chemically active or activated derivatives of the PEG polymer are prepared to attach the PEG to the desired molecule.

The overall PEGylation processes used to date for protein conjugation can be broadly classified into two types, namely a solution phase batch process and an on-column fed-batch process. The simple and commonly adopted batch process involves the mixing of reagents together in a suitable buffer solution, preferably at a temperature between 4 and 6° C., followed by the separation and purification of the desired product using a suitable technique based on its physicochemical properties, including size exclusion chromatography (SEC), ion exchange chromatography (IEX), hydrophobic interaction chromatography (HIC) and membranes or aqueous two phase systems.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, tyrosine. The N-terminal amino group and the C-terminal carboxylic acid can also be used as a site specific site by conjugation with aldehyde functional polymers.

The techniques used to form first generation PEG derivatives are generally reacting the PEG polymer with a group that is reactive with hydroxyl groups, typically anhydrides, acid chlorides, chloroformates and carbonates. In the second generation PEGylation chemistry more efficient functional groups such as aldehyde, esters, amides etc made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible and biocompatible spacer is needed. Preferred end groups for heterobifunctional PEGs are maleimide, vinyl sulfones, pyridyl disulfide, amine, carboxylic acids and NHS esters.

Third generation pegylation agents, where the shape of the polymer has been branched, Y shaped or comb shaped are available which show reduced viscosity and lack of organ accumulation. U.S. Pat. No. 8,007,784 (Scott) shows a specific process or pegylation even to blood cells that is sufficiently mild as to increase survivability of stored cells.

End groups listed above for pegylation also include some reactive groups for the other reactions (e.g., hydroxy groups, carboxylic acid groups, amines, vinyl compounds, ethylenically unsaturated groups, acrylic groups, silanes and the like).

### DNA-Binding Components

In certain embodiments, the compositions and methods disclosed herein involve fusions between a DNA-binding domain and restriction endonucleases. A DNA-binding domain can comprise any molecular entity capable of sequence-specific binding to chromosomal DNA. Binding can be mediated by electrostatic interactions, hydrophobic interactions, hydrogen bonding or any other type of physiochemical force. Examples of moieties which can comprise part of a DNA-binding domain include, but are not limited to, minor groove binders, major groove binders, antibiotics, intercalating agents, peptides, polypeptides, oligonucleotides, and nucleic acids. An example of a DNA-binding nucleic acid is a triplex-forming oligonucleotide.

Minor groove binders include substances, which by virtue of their steric and/or electrostatic properties, interact preferentially with the minor groove of double-stranded nucleic acids. Certain minor groove binders exhibit a preference for particular sequence compositions. For instance, netropsin, distamycin and CC-1065 are examples of minor groove binders, which bind specifically to AT-rich sequences, particularly runs of A or T. WO 96/32496.

Many antibiotics are known to exert their effects by binding to DNA. Binding of antibiotics to DNA is often sequence-specific or exhibits sequence preferences. Actinomycin, for instance, is a relatively GC-specific DNA binding agent. Synthetic oligonucleotides could also be used to target specific regions of DNA.

In a preferred embodiment, a DNA-binding domain is a polypeptide. Certain peptide and polypeptide sequences bind to double-stranded DNA in a sequence-specific manner. For example, transcription factors participate in transcription initiation by sequence-specific interactions with DNA in the promoter and/or enhancer regions of genes, which recruit RNA Polymerase II. Defined regions within the polypeptide sequence of various transcription factors have been shown to be responsible for sequence-specific binding to DNA. See, for example, Pabo et al. (1992) Ann. Rev. Biochem. 61:1053-1095 and references cited therein. These regions include, but are not limited to, motifs known as helix-loop-helix (HLH) domains, helix-turn-helix domains, zinc fingers, β-sheet motifs, steroid receptor motifs, bZIP domains homeodomains, AT-hooks and others. The amino acid sequences of these motifs are known and, in some cases, amino acids that are critical for sequence specificity have been identified. Polypeptides involved in other processes involving DNA, such as replication, recombination and repair, will also have regions involved in specific interactions with DNA. Peptide sequences involved in specific DNA recognition, such as those found in transcription factors, can be obtained through recombinant DNA cloning and expression techniques or by chemical synthesis, and can be attached to other components of a fusion molecule by methods known in the art.

Proteins containing methyl binding domains, or functional fragments thereof, can also be used as DNA-binding domains. Methyl binding domain proteins recognize and bind to CpG dinucleotide sequences in which the C nucleotide base is methylated. Proteins containing a methyl-binding domain include, but are not limited to, MBD1, MBD2, MBD3, MBD4, MeCP1 and MeCP2. See, for example, Bird et al. (1999) Cell 99:451-454.

Additionally, DNA methyl transferases, which methylate the 5-position of C residues in CpG dinucleotides such as, for example, DNMT1, DNMT2, DNMT3a and DNMT3b, or functional fragments thereof, can be used as a DNA-binding domain. Furthermore, enzymes which demethylate methylated CpG, or functional fragments thereof, can be used as a DNA-binding domain. Fremant et al. (1997) Nucleic Acids Res. 25:2375-2380; Okano et al (1998) Nature Genet. 19:219-220; Bhattacharya et al. (1999) Nature 397:579-583; and Robertson et al. (2000) Carcinogenesis 21:461-467.

In one more embodiment, a DNA-binding domain may comprise a zinc finger DNA-binding domain. See, for example, Miller et al. (1985) EMBO J. 4:1609-1614; Rhodes et al. (1993) Scientific American February: 56-65; and Klug (1999) J. Mol. Biol. 293:215-218. In one embodiment, a target site for a zinc finger DNA-binding domain is identified according to site selection rules disclosed in co-owned WO 00/42219. ZFP DNA-binding domains are designed and/or selected to recognize a particular target site as described in co-owned WO 00/42219; WO 00/41566; and U.S. Ser. No. 09/444,241 filed Nov. 19, 1999 and Ser. No. 09/535,088 filed Mar. 23, 2000; as well as U.S. Pat. Nos. 5,189,538; 6,007,408; and 6,013,453; and PCT publications WO 95/19431, WO 98/54311, WO 00/23464 and WO 00/27878.

Certain DNA-binding domains are capable of binding to DNA that is packaged in nucleosomes. See, for example, Cordingley et al. (1987) Cell 48:261-270; Pina et al. (1990) Cell 60:719-731; and Cirillo et al. (1998) EMBO J. 17:244-254. Certain ZFP-containing proteins such as, for example, members of the nuclear hormone receptor superfamily, are capable of binding DNA sequences packaged into chromatin. These include, but are not limited to, the glucocorticoid receptor and the thyroid hormone receptor. Archer et al. (1992) Science 255:1573-1576; Wong et al. (1997) EMBO J. 16:7130-7145. Other DNA-binding domains, including certain ZFP-containing binding domains, require more accessible DNA for binding. In the latter case, the binding specificity of the DNA-binding domain can be determined by identifying accessible regions in the cellular chromatin. Accessible regions can be determined as described in co-owned U.S. patent application entitled “Databases of Accessible Region Sequences; Methods of Preparation and Use Thereof,” reference S15, filed even date herewith, the disclosure of which is hereby incorporated by reference herein. A DNA-binding domain is then designed and/or selected to bind to a target site within the accessible region.

### Endonuclease Components

The following list of restriction enzymes or restriction endonucleases and enzymes sorted by target or defective sequences (prepared by Bruce Williams, New England BioLabs) is provided as evidence of the known and available skill of the ordinary artisan in the present field of technology to select appropriate enzymes for specific target sequences in the preparation of P2E2 constructs according to the present technology.

I) Alphabetic list of restriction enzymes
II) Enzymes sorted by target or defective sequence

### Nucleotide Symbols Used:

### R=A or G M=A or C H=A, C or T N=A, C, G or T

### Y=C or T K=G or T V=A, C or G

S=C or G B=C, G or T
W=A or T D=A, G or T

### I) Restriction Endonucleases Listed Alphabetically by Name

Note: Position of cleavage indicated by / or (number).

i.e.,: (3)ACGT==/NNNACGT.

i.e., ACGT(5)==ACGTNNNNN/

| Enzyme | DNA |
|---|---|
| Name | Target |
| AatI | AGG/CCT |
| AatII | GACGT/C |
| AccI | GT/MKAC |
| AccIII | T/CCGGA |
| Acc65I | G/GTACC |
| AciI | C/CGC and G/CGG |
| AcsI | R/AATTY |
| AcyI | GR/CGYC |
| AflI | G/GWCC |
| AflII | C/TTAAG |
| AflIII | A/CRYGT |
| AgeI | A/CCGGT |
| AhaII | GR/CGYC |
| AhaIII | TTT/AAA |
| AluI | AG/CT |
| AlwI | GGATC(4) and (5)GATCC |
| Alw44I | G/TGCAC |
| AlwNI | CAGNNN/CTG |
| AocI | CC/TNAGG |
| AosI | TGC/GCA |
| ApaI | GGGCC/C |
| ApaLI | G/TGCAC |
| ApoI | R/AATTY |
| ApyI | /CCWGG |
| AscI | GG/CGCGCC |
| AseI | AT/TAAT |
| AsnI | AT/TAAT |
| AspI | GACN/NNGTC |
| Asp700 | GAANN/NNTTC |
| Asp718 | G/GTACC |
| AspEI | GACNNN/NNGTC |
| AspHI | GWGCW/C |
| AsuII | TT/CGAA |
| AvaI | C/YCGRG |
| AvaII | G/GWCC |
| AviII | TGC/GCA |
| AvrII | C/CTAGG |
| BalI | TGG/CCA |
| BamHI | G/GATCC |
| BanI | G/GYRCC |
| BanII | GRGCY/C |
| BbrPI | CAC/GTG |
| BbsI | GAAGACNN/ and (6)GTCTTC |
| BbvI | GCAGC(8) and (12)GCTGC |
| BcgI | (10)CGANNNNNNTGC(12) and |
| | (10)GCANTCG(12) |
| BclI | T/GATCA |
| BfaI | C/TAG |
| BfrI | C/TTAAG |
| BglI | GCCNNNN/NGGC |
| BglII | A/GATCT |
| BinI | C/CTAGG |
| BmyI | GDGCH/C |
| BpmI | CTGGAG(16) and (14)CTCCAG |
| BpuAI | GAAGACNN/ and (6)GTCTTC |
| Bpu1102I | GC/TNAGC |
| BsaI | GGTCTCN/ and (5)GAGACC |
| BsaAI | YAC/GTR |
| BsaBI | GATNN/NNATC |
| BsaHI | GR/CGYC |
| BsaJI | C/CNNGG |
| BseAI | T/CCGGA |
| BsePI | G/CGCGC |
| BsgI | GTGCAG(16) and (14)CTGCAC |
| BsiEI | CGRY/CG |
| BsiWI | C/GTACG |
| BsiYI | CCNNNNN/NNGG |
| BslI | CC/NNGG |
| BsmI | GAATGCN/ and /NGCATTC |
| BsmAI | GTCTCN/ and (5)GAGAC |
| Bsp1286I | GDGCH/C |
| Bsp1407I | T/GTACA |
| BspDI | AT/CGAT |
| BspEI | T/CCGGA |
| BspHI | T/CATGA |
| BspLU11I | A/CATGT |
| BspMI | ACCTGC(4) and (8)GCAGGT |
| BsrI | ACTGGN/ and /NCCAGT |
| BsrFI | R/CCGGY |
| BssGI | CCA/NTGG |
| BssHII | G/CGCGC |
| Bst1107I | GTA/TAC |
| BstBI | TT/CGAA |
| BstEII | G/GTNACC |
| BstNI | CC/WGG |
| BstUI | CG/CG |
| BstXI | CCANNNNN/NTGG |
| BstYI | R/GATCY |
| Bsu36I | CC/TNAGG |
| CelII | GC/TNAGC |
| CfoI | GCG/C |
| CfrI | Y/GGCCR |
| Cfr10I | R/CCGGY |
| Clal | AT/CGAT |
| DdeI | C/TNAG |
| DpnI | GA/TC (only if G methylated) |
| DpnII | /GATC |
| Dral | TTT/AAA |
| DraII | RG/GNCCY |
| DraIII | CACNNN/GTG |
| DrdI | GACNNNN/NNGTC |
| DsaI | C/CRYGG |
| EaeI | Y/GGCCR |
| EagI | C/GGCCG |
| Eam1105I | GACNNN/NNGTC |
| EarI | CTCTTCN/ and (4)GAAGAG |
| Ecl136II | GAG/CTC |
| EclXI | C/GGCCG |
| Eco47III | AGC/GCT |
| Eco57I | CTGAAG(16) and (14)CTTCAG |
| EcoNI | CCTNN/NNNAGG |
| EcoO109I | RG/GNCCY |
| EcoRI | G/AATTC |
| EcoRII | /CCWGG |
| EcoRV | GAT/ATC |
| EspI | GC/TNAGC |
| Esp3I | CGTCTCN/ and (5)GAGACG |
| FnuDII | CG/CG |
| Fnu4HI | GC/NGC |
| FokI | GGATG(9) and (13)CATCC |
| FseI | GGCCGG/CC |
| FspI | TGC/GCA |
| GsuI | CTGGAG(16) and (14)CTCCAG |
| HaeII | RGCGC/Y |
| HaeIII | GG/CC |
| HgaI | GACGC(5) and (10)GCGTC |
| HgiAI | GWGCW/C |
| HhaI | GCG/C |
| HincII | GTY/RAC |
| HindII | GTY/RAC |
| HindIII | A/AGCTT |
| HinfI | G/ANTC |
| HinPI | G/CGC |
| HpaI | GTT/AAC |
| HpaII | C/CGG |
| HphI | GGTGA(8) and (7)TCACC |
| ItaI | GC/NGC |
| KasI | G/GCGCC |
| KpnI | GGTAC/C |
| KspI | CCGC/GG |
| MaeI | C/TAG |
| MaeII | A/CGT |
| MaeIII | /GTNAC |
| MamI | GATNN/NNATC |
| MboI | /GATC |
| MboII | GAAGA(8) and (7)TCTTC |
| MfeI | C/AATTG |
| MluI | A/CGCGT |
| MluNI | TGG/CCA |
| MnlI | CCTC(7) and (6)GAGG |
| MroI | T/CCGGA |
| MscI | TGG/CCA |
| MseI | T/TAA |
| MspI | C/CGG |
| MstI | TGC/GCA |
| MstII | CC/TNAGG |
| MunI | C/AATTG |
| MvaI | CC/WGG |
| MvnI | CG/CG |
| NaeI | GCC/GGC |
| NarI | GG/CGCC |
| NciI | CC/SGG |
| NcoI | C/CATGG |
| NdeI | CA/TATG |
| NdeII | /GATC |
| NgoMI | G/CCGGC |
| NheI | G/CTAGC |
| NlaIII | CATG/ |
| NlaIV | GGN/NCC |
| NotI | GC/GGCCGC |
| NruI | TCG/CGA |
| NsiI | ATGCA/T |
| NspBII | CMG/CKG |
| NspI | RCATG/Y |
| NspII | GDGCH/C |
| NspV | TT/CGAA |
| PacI | TTAAT/TAA |
| PaeR7I | C/TCGAG |
| PflMI | CCANNNN/NTGG |
| PinAI | A/CCGGT |
| PleI | GAGTC(4) and (5)GACTC |
| PmaCI | CAC/GTG |
| PmeI | GTTT/AAAC |
| PmlI | CAC/GTG |
| PpuMI | RG/GWCCY |
| Psp1406I | AA/CGTT |
| PstI | CTGCA/G |
| PvuI | CGAT/CG |
| PvuII | CAG/CTG |
| RcaI | T/CATGA |
| RmaI | C/TAG |
| RsaI | GT/AC |
| RsrII | CG/GWCCG |
| Sad | GAGCT/C |
| SacII | CCGC/GG |
| SalI | G/TCGAC |
| SauI | CC/TNAGG |
| Sau3AI | /GATC |
| Sau96I | G/GNCC |
| ScaI | AGT/ACT |
| ScrFI | CC/NGG |
| SexAI | A/CCWGGT |
| SfaNI | GCATC(5) and (9)GATGC |
| SfcI | C/TRYAG |
| SfiI | GGCCNNNN/NGGCC |
| SfuI | TT/CGAA |
| SgrAI | CR/CCGGYG |
| SmaI | CCC/GGG |
| SnaBI | TAC/GTA |
| SnoI | G/TGCAC |
| SpeI | A/CTAGT |
| SphI | GCATG/C |
| SrfI | GCCC/GGGC |
| Sse8387I | CCTGCA/GG |
| SspI | AAT/ATT |
| SspBI | T/GTACA |
| SstI | GAGCT/C |
| SstII | CCGC/GG |
| StuI | AGG/CCT |
| StyI | C/CWWGG |
| SwaI | ATTT/AAAT |
| TaqI | T/C GA |
| TfiI | G/AWTC |
| ThaI | CG/CG |
| Tru9I | T/TAA |
| Tth111I | GACN/NNGTC |
| Van91I | CCANNNN/NTGG |
| XbaI | T/CTAGA |
| XcmI | C CA/NNNNTGG |
| XhoI | C/TCGAG |
| XhoII | R/GATCY |
| XmaI | C/CCGGG |
| XmaIII | C/GGCCG |
| XmaCI | C/CCGGG |
| XmnI | GAANN/NNTTC |

Note: Position of cleavage indicated by / or (number).

### i.e.,: (3)ACGT==/NNNACGT.

### i.e., ACGT(5)==ACGTNNNNN/

### II) Restriction Endonucleases Arranged by Target Sequence

Target sequences are grouped by first 2 characters:

AA AC AG AT
CA CC CG CT
GA GC GG GT
TA TC TG TT

Note: Numbers in parentheses indicate position of cleavage. The first number refers to the strand containing the motif cited; the second refers to the complementary strand. Thus ACNGA(1,5) indicates: ACNGAN/TGNCTNNNNN/

| | | |
|---|---|---|
| Sequence cut | Enzyme Name | notes |

AA

| | |
|---|---|
| R/AATTY | AcsI |
| R/AATTY | ApoI |
| A/AGCTT | HindIII |
| AA/CGTT | Psp1406I |
| AAT/ATT | SspI |

AC

| | |
|---|---|
| A/CRYGT | AflIII |
| A/CCGGT | AgeI |
| A/CATGT | BspLU11I |
| ACCTGC(4,8) | BspMI |
| ACTGG(1,−1) | BsrI |
| R/CCGGY | BsrFI |
| R/CCGGY | Cfr10 |
| A/CGT | MaeII |
| A/CGCGT | MluI |
| RCATG/Y | NspI |
| A/CCGGT | PinAI |
| A/CCWGGT | SexAI |
| A/CTAGT | SpeI |

AG

| | |
|---|---|
| AGG/CCT | AatI |
| AG/CT | AluI |
| A/GATCT | BglII |
| R/GATCY | BstYI |
| RG/GNCCY | DraII |
| AGC/GCT | Eco47III |
| RG/GNCCY | EcoO109I |
| RGCGC/Y | HaeII |
| RG/GWCCY | PpuMI |
| AGT/ACT | ScaI |
| AGG/CCT | StuI |
| R/GATCY | XhoII |

AT

| | |
|---|---|
| AT/TAAT | AseI |
| AT/TAAT | AsnI |
| AT/CGAT | BspDI |
| AT/CGAT | ClaI |
| ATGCA/T | NsiI |
| ATTT/AAAT | SwaI |

CA

| | |
|---|---|
| CAGNNN/CTG | AlwNI |
| CAC/GTG | BbrPI |
| CACNNN/GTG | DraIII |
| YAC/GTR | BsaAI |
| C/AATTG | MfeI |
| C/AATTG | MunI |
| CA/TATG | NdeI |
| CATG/ | NlaIII |
| CMG/CKG | NspBII |
| CAC/GTG | PmaCI |
| CAC/GTG | PmlI |
| CAG/CTG | PvuII |
| CR/CCGGYG | SgrAI |
| (13,9)CATCC | FokI |

CC

| | |
|---|---|
| C/CGC | AciI |
| CC/TNAGG | AocI |
| /CCWGG | ApyI |
| C/CTAGG | AvrII |
| C/CTAGG | BinI |
| C/CNNGG | BsaJI |
| CC/NNGG | BsiYI |
| CC/NNGG | BslI |
| CCA/NTGG | BssGI |
| CC/WGG | BstNI |
| CCA/NTGG | BstXI |
| CC/TNAGG | Bsu36I |
| C/CRYGG | DsaI |
| C/YCGRG | Aval |
| CC TNN/NNNAGG | EcoNI |
| /CCWGG | EcoRII |
| C/CGG | HpaII |
| CCGC/GG | KspI |
| CCTC(7,6) | MnlI |
| C/CGG | MspI |
| CC/TNAGG | MstII |
| CC/WGG | MvaI |
| CC/SGG | NciI |
| C/CATGG | NcoI |
| CMG/CKG | NspBII |
| CCANNNN/NTGG | PflMI |
| CCGC/GG | SacII |
| CC/TNAGG | SauI |
| CC/NGG | ScrFI |
| CCC/GGG | Smal |
| CCTGCA/GG | Sse83871 |
| CCGC/GG | SstII |
| C/CWWGG | StyI |
| CCANNNN/NTGG | Van91I |
| CCA/NNNNTGG | XcmI |
| C/CCGGG | XmaI |
| C/CCGGG | XmaCI |
| (1,−1)CCAGT | BsrI |

CG

| | |
|---|---|
| (10,12)CGANNNNNNTGC(12,10) | BcgI |
| CGRY/CG | BsiEI |
| C/GTACG | BsiWI |
| CG/CG | BstUI |
| C/GGCCG | EagI |
| Y/GGCCR | CfrI |
| Y/GGCCR | EaeI |
| C/GGCCG | EclXI |
| CGTCTC(1,5) | Esp3I |
| CG/CG | FnuDII |
| CG/CG | MvnI |
| CGAT/CG | PvuI |
| CG/GWCCG | RsrII |
| CR/CCGGYG | SgrAI |
| CG/CG | ThaI |
| C/GGCCG | XmaIII |

CT

| | |
|---|---|
| C/TTAAG | AflII |
| C/TAG | BfaI |
| C/TTAAG | BfrI |
| CTGGAG(16,14) | BpmI |
| C/TNAG | DdeI |
| CTCTTC(1,4) | EarI |
| C/YCGRG | AvaI |
| CTGAAG(16,14) | Eco57I |
| CTGGAG(16,14) | GsuI |
| C/TAG | MaeI |
| C/TCGAG | PaeR7I |
| CTGCA/G | PstI |
| C/TAG | RmaI |
| C/TRYAG | SfcI |
| C/TCGAG | XhoI |
| (14,16)CTCCAG | BpmI |
| (14,16)CTGCAC | BsgI |
| (14,16)CTTCAG | Eco57I |
| (14,16)CTCCAG | GsuI |

GA

| | |
|---|---|
| GACGT/C | AatII |
| GACN/NNGTC | AspI |
| GAANN/NNTTC | Asp700 |
| GACNNN/NNGTC | AspEI |
| GAAGAC(2,6) | BbsI |
| GAAGAC(2,6) | BpuAI |
| GATNN/NNATC | BsaBI |
| GAATGC(1,-1) | Bsm1 |
| GA/TC | DpnI only if G-Me |
| /GATC | DpnII |
| GACNNNN/NNGTC | DrdI |
| GACNNN/NNGTC | Eam1105I |
| GAG/CT C | Ec113611 |
| R/AATTY | AcsI |
| GR/CGYC | AcyI |
| GR/CGYC | AhaII |
| R/AATTY | ApoI |
| GWGCW/C | AspHI |
| GRGCY/C | BanII |
| GDGCH/C | BmyI |
| GR/CGYC | BsaHI |
| GDGCH/C | Bsp1286I |
| G/AATTC | EcoRI |
| GAT/ATC | EcoRV |
| GACGC(5,10) | HgaI |
| GWGCW/C | HgiAI |
| G/ANTC | HinfI |
| GATNN/NNATC | MamI |
| /GATC | MboI |
| GAAGA(8,7) | MboII |
| /GATC | NdeII |
| GDGCH/C | NspII |
| GAGT C(4,5) | PleI |
| GAGCT/C | SacI |
| /GATC | Sau3AI |
| GAGCT/C | SstI |
| G/AWTC | TfiI |
| GACN/NNGTC | Tth111I |
| GAANN/NNTTC | XmnI |
| (9,5)GATGC | SfaNI |
| (5,4)GATCC | AlwI |
| (5,1)GAGACC | BsaI |
| (5,1)GAGAC | BsmAI |
| (4,1)GAAGAG | EarI |
| (5,1)GAGACG | Esp3I |
| (6,7)GAGG | MnlI |
| (5,4)GACTC | Plel |

GC

| | |
|---|---|
| GCAGC(8,12) | BbvI |
| GC CNNNN/NGGC | BglI |
| GC/TNAGC | Bpu1102I |
| G/CGCGC | BsePI |
| G/CGCGC | BssHII |
| GC/TNAGC | CelII |
| GCG/C | CfoI |
| R/CCGGY | BsrFI |
| R/CCGGY | Cfr10I |
| GC/TNAGC | EspI |
| GC/NGC | Fnu4HI |
| GCG/C | HhaI |
| G/CGC | HinPI |
| GC/NGC | Ital |
| GCC/GGC | NaeI |
| G/CCGGC | NgoMI |
| G/CTAGC | NheI |
| GC/GGCCGC | NotI |
| RCATG/Y | NspI |
| GCATC(5,9) | SfaNI |
| GCATG/C | SphI |
| GCCC/GGGC | Srfl |
| G/CGG | AciI |
| (12,8)GCTGC | BbvI |
| (10,12)GCANNNNNNTCG(12,10) | BcgI |
| (1,1)GCATTC | BsmI |
| (8,4)GCAGGT | BspMI |
| (10,5)GCGTC | HgaI |

GG

| | |
|---|---|
| G/GTACC | Acc65I |
| G/GWCC | AflI |
| GGATC(4,5) | AlwI |
| GGGCC/C | ApaI |
| GG/CGCGCC | AscI |
| G/GTACC | Asp718 |
| G/GWCC | AvaII |
| G/GATCC | BamHI |
| G/GYRCC | BanI |
| GGTCTC(1,5) | BsaI |
| G/GTNACC | BstEII |
| R/GATCY | BstYI |
| GR/CGYC | AcyI |
| GR/CGYC | AhaII |
| GRGCY/C | BanII |
| GDGCH/C | BmyI |
| GR/CGYC | BsaHI |
| GDGCH/C | Bsp12861 |
| RG/GNCCY | DraII |
| RG/GNCCY | EcoO109I |
| GGATG(9,13) | FokI |
| GGCCGG/CC | FseI |
| RGCGC/Y | HaeII |
| GG/CC | HaeIII |
| GGTGA(8,7) | HphI |
| G/GCGCC | KasI |
| GGTAC/C | KpnI |
| GG/CGCC | Nan |
| GGN/NCC | NlaIV |
| GDGCH/C | NspII |
| RG/GWCCY | PpuMI |
| G/GNCC | Sau96I |
| GGCCNNNN/NGGCC | SfiI |
| R/GATCY | XhoII |

GT

| | |
|---|---|
| GT/MKAC | AccI |
| G/TGCAC | Alw441 |
| G/TGCAC | ApaLI |
| GTGCAG(16,14) | BsgI |
| GTCTC(1,5) | BsmAI |
| GTA/TAC | Bst1107I |
| GWGCW/C | AspHI |
| GDGCH/C | BmyI |
| GDGCH/C | Bsp12861 |
| GWGCW/C | HgiAI |
| GTY/RAC | HincII |
| GTY/RAC | HindII |
| GTT/AAC | HpaI |
| /GTNAC | MaeIII |
| GDGCH/C | NspII |
| GTTT/AAAC | PmeI |
| GT/AC | RsaI |
| G/TCGAC | SalI |
| G/TGCAC | SnoI |
| (6,2)GTCTTC | BbsI |
| (6,2)GTCTTC | BpuAI |

TA

| | |
|---|---|
| YAC/GTR | BsaAI |
| TAC/GTA | SnaBI |

TC

| | |
|---|---|
| T/CCGGA | AccII |
| T/CCGGA | BseAI |
| T/CCGGA | BspEI |
| T/CATGA | BspHI |
| T/CCGGA | MroI |
| TCG/CGA | NruI |
| T/CATGA | RcaI |
| T/CGA | TaqI |
| T/CTAGA | XbaI |
| (7,8)TCACC | HphI |
| (7,8)TCTTC | MboII |

TG

| | |
|---|---|
| TGC/GCA | AosI |
| TGC/GCA | AviII |
| TGG/CCA | BalI |
| T/GATCA | BelI |
| T/GTACA | Bsp1407I |
| Y/GGCCR | CfrI |
| Y/GGCCR | EaeI |
| TGC/GCA | FspI |
| TGG/CCA | MluNI |
| TGG/CCA | MscI |
| TGC/GCA | MstI |
| T/GTACA | SspBI |

TT

| | |
|---|---|
| TTT/AAA | AhaIII |
| TT/CGAA | AsuII |
| TT/CGAA | BstBI |
| TTT/AAA | DraI |
| T/TAA | MseI |
| TT/CGAA | NspV |
| TTAAT/TAA | PacI |
| TT/CGAA | SfuI |
| T/TAA | Tru9I |

Numbers in parentheses indicate position of cleavage. The first number refers to the strand containing the motif cited; the second refers to the complementary strand. Thus ACNGA(1,5) indicates: ACNGAN/TGNCTNNNNN/

### P2E2 Construct Synthesis

The three components of the P2E2 construct may be connected by various molecular biology chemical reactions referred to as gene synthesis, polymerase chain reaction, and subcloning easily performed by those skilled in the art. As the two-part DNA binder and Restriction Endonuclease construct is already known, it is easiest to explain the techniques for making the three-part P2E2 construct beginning with that commercially available intermediate. A free end of one of the two segments may be provided with a reactive site or pendant group A. The cell-penetration segment is then provided with a corresponding reactive site or pendant group B. By reacting A and B, the third segment is appropriately added to form the three-part P2E2 construct.

A preferred method of forming the P2E2 construct includes the use of recombinant DNA and molecular cloning to encode 1, 2 or 3 segments of the three-part P2E2 construct. Molecular cloning is the laboratory process used to create recombinant DNA. It is one of two basic methods (along with polymerase chain reaction, PCR) used to direct the replication of any specific DNA sequence chosen. The fundamental difference between the two methods is that molecular cloning involves replication of the DNA within a living cell, while PCR replicates DNA in a machine, free of living cells.

Formation of recombinant DNA requires a cloning vector such as a plasmid, cosmid, bacterial artificial chromosomes (BACs), or other DNA molecule that will replicate within a living cell. Vectors are generally derived from plasmids, and represent relatively small segments of DNA that contain necessary genetic signals for replication, as well as additional elements for convenience in inserting foreign DNA, identifying cells that contain recombinant DNA, and, where appropriate, expressing the foreign DNAas and RNA and protein. The choice of plasmid vector for molecular cloning depends on the choice of host organism, the size of the DNA to be cloned, and whether and how the foreign DNA is to be expressed. The DNA segments can be combined by using a variety of methods, such as restriction enzyme/ligase cloning or Gibson assembly.

In standard cloning protocols, the cloning of any DNA fragment essentially involves seven steps: (1) Choice of host organism and cloning vector, (2) Preparation of plasmid vector DNA, (3) Preparation of DNA to be cloned, (4) Creation of recombinant DNA, (5) Introduction of recombinant DNA into the host organism, (6) Selection of organisms containing the recombinant DNA, (7) Screening for clones with desired DNA inserts and biological properties and DNA sequencing to verify the correct recombinant. These steps are described below.

1) Choice of Host Organism and Cloning Vector

Although a very large number of host organisms and molecular cloning vectors are in use, the great majority of molecular cloning efforts begin with a laboratory strain of the bacterium E. coli (Escherichia coli) and a plasmid cloning vector. E. coli and plasmid vectors are in common use because they are technically sophisticated, versatile, widely available, and offer rapid growth of recombinant organisms with minimal equipment. The scope of the invention is not limited by this preferential use of E. coli. If the DNA to be cloned is exceptionally large (hundreds of thousands to millions of base pairs), then a bacterial artificial chromosome (BAC) or yeast artificial chromosome (YAC) vector is often chosen.

Specialized applications may call for specialized host-vector systems. For example, if the experimentalists wish to harvest a particular protein from the recombinant organism, then an expression vector is chosen that contains appropriate signals for transcription and translation in the desired host organism. Alternatively, if replication of the DNA in different species is desired (for example transfer of DNA from bacteria to plants), then a multiple host range vector (also termed shuttle vector) may be selected. In practice, however, specialized molecular cloning experiments usually begin with cloning into a bacterial plasmid, followed by subcloning into a specialized vector.

Whatever combination of host and vector are used, the vector often contains four DNA segments that are important to its function and experimental utility—(1) an origin of DNA replication is necessary for the vector (and recombinant sequences linked to it) to replicate inside the host organism, (2) one or more unique restriction endonuclease recognition sites that serves as sites where foreign DNA may be introduced, (3) a selectable genetic marker gene that can be used to enable the survival of cells that have taken up vector sequences, and (4) an additional gene that can be used for screening which cells contain foreign DNA. The fourth component is the least critical within the scope of practice of the present invention.

2. Preparation of Vector DNA

The purified cloning vector is treated with one or more restriction endonucleases to cleave the DNA at the site where foreign DNA will be inserted. The restriction enzymes are chosen to generate a configuration at the cleavage site that is compatible with that at the ends of the foreign DNA. Typically, this is done by cleaving the vector DNA and foreign DNA with the same restriction enzymes, for example EcoRI. Most modern vectors contain a variety of convenient cleavage sites (multiple cloning site) that are unique within the vector molecule (so that the vector can only be cleaved at a single site by these enzymes) and is located within a reporter gene (frequently beta-galactosidase) whose inactivation can be used to distinguish recombinant from non-recombinant organisms at a later screening step in the process. To improve the ratio of recombinant to non-recombinant organisms, the cleaved vector may be treated with an enzyme (alkaline phosphatase) that dephosphorylates the vector ends. Linear Vector molecules are not able to replication, so treatment of linearized vectors to dephosphorylated ends prevents closing to a circular plasmid, and thus is unable to replicate, and replication can only be restored if foreign DNA is integrated into the cleavage site allowing closing and cirularization of the recombinant plasmid.

3. Preparation of DNA to be Cloned

For cloning of genomic DNA, the DNA to be cloned may be extracted from the organism of interest. Virtually any tissue source can be used (even tissues from extinct animals), as long as the DNA is not extensively degraded. The DNA is then purified using simple methods to remove contaminating proteins (extraction with phenol), RNA (ribonuclease) and smaller molecules (precipitation and/or chromatography). Polymerase chain reaction (PCR) methods are often used for amplification of specific DNA or RNA (RT-PCR) sequences prior to molecular cloning.

DNA for cloning experiments may also be obtained from RNA using reverse transcriptase (complementary DNA or cDNA cloning), or in the form of synthetic DNA (artificial gene synthesis). cDNA cloning is usually used to obtain clones representative of the mRNA population of the cells of interest, while synthetic DNA is used to obtain any precise sequence defined by the designer. Both can be used to generate sequences used for protein expression.

The purified DNA is then treated with a restriction enzyme to generate fragments with ends capable of being linked to those of the vector. If necessary, short double-stranded segments of DNA (linkers) containing desired restriction sites may be added to create end structures that are compatible with the vector.

4. Creation of Recombinant DNA with DNA Ligase

The creation of recombinant DNA is in many ways the simplest step of the molecular cloning process. DNA prepared from the vector and foreign DNA source are simply mixed together at appropriate concentrations and exposed to an enzyme (DNA ligase) under specific conditions that covalently joins the ends together forming a circularized molecule. This joining reaction is often termed ligation. The resulting DNA mixture containing randomly joined ends is then ready for introduction into the host organism.

DNA ligase only recognizes and acts on the ends of linear DNA molecules, usually resulting in a complex mixture of DNA molecules, some with randomly joined ends. The desired products (vector DNA covalently linked to foreign DNA) will be present, but other sequences (e.g. foreign DNA linked to itself, vector DNA linked to itself and higher-order combinations of vector and foreign DNA) are also usually present. This complex mixture is sorted out in subsequent steps of the cloning process, after the DNA mixture is introduced into cells.

5. Introduction of Recombinant DNA into the Host Organism

The DNA mixture, previously manipulated in vitro, is moved back into a living cell, referred to as the host organism. The methods used to get DNA into cells are varied, and the name applied to this step in the molecular cloning process will often depend upon the experimental method that is chosen (e.g., transformation, transduction, transfection and/or electroporation).

When microorganisms are able to take up and replicate DNA from their local environment, the process is termed transformation, and cells that are in a physiological state such that they can take up DNA are said to be competent. In mammalian cell culture, the analogous process of introducing DNA into cells is commonly termed transfection. Both transformation and transfection usually require preparation of the cells through a special growth regime and chemical treatment process that will vary with the specific species and cell types that are used. Bacterial transformation is almost always used for cloning.

Electroporation uses high voltage electrical pulses to translocate DNA across the cell membrane (and cell wall, if present). In contrast, transduction involves the packaging of DNA into virus-derived particles, and using these virus-like particles to introduce the encapsulated DNA into the cell through a process resembling viral infection. All of these methods are commonly used in the laboratory setting.

6. Selection of Organisms Containing Vector Sequences

Which ever method is used, the introduction of recombinant DNA into the chosen host organism is usually a low efficiency process; that is, only a small fraction of the cells will actually take up DNA. Experimental scientists deal with this issue through a step of artificial genetic selection, in which cells that have not taken up DNA are selectively killed, and only those cells that can actively replicate DNA containing the selectable marker gene encoded by the vector are able to survive.

When bacterial cells are used as host organisms, the selectable marker is usually a gene that confers resistance to an antibiotic that would otherwise kill the cells, typically ampicillin. Cells harboring the vector will survive when exposed to the antibiotic, while those that have failed to take up vector sequences will die. When mammalian cells (e.g., human or mouse cells) are used, a similar strategy is used, except that the marker gene confers resistance to a poison such as Geneticin, puromycin or hyromycin and the like.

7. Screening for Clones with Desired DNA Inserts and Biological Properties and DNA Sequencing to Verify the Correct Recombinant

Modern bacterial cloning vectors (e.g., pUC19 and later derivatives including the pGEM vectors) use the blue-white screening system to distinguish colonies (clones) of transgenic cells from those that contain the parental vector (i.e., vector DNA with no recombinant sequence inserted). In these vectors, foreign DNA is inserted into a sequence that encodes the beta-galactosidase protein, an enzyme whose activity results in formation of a blue-colored colony on the culture medium containing the x-Gal substrate. Insertion of the foreign DNA into the beta-galactosidase coding sequence, disrupts the correct reading frame, and produces a protein lacking beta-galactosidase enzymatic activity, so that resulting bacterial colonies containing these recombinant plasmids remain colorless (white). Therefore, experimentalists are easily able to identify and conduct further studies on transgenic bacterial clones, while ignoring those that do not contain recombinant DNA.

When multiple different DNA molecules are cloned in the same experiment, it is almost always necessary to examine a number of different clones to be sure that the desired DNA construct is obtained. This may be accomplished through a very wide range of experimental methods, including the use of nucleic acid hybridizations, antibody probes, polymerase chain reaction, and/or restriction fragment analysis. DNA sequencing is used as the standard method to validate that the desired recombinant construct was accurately made.

### Generic P2E2 Three Component Construct

To build a generic three component P2E2 construct, the following scheme can be applied.

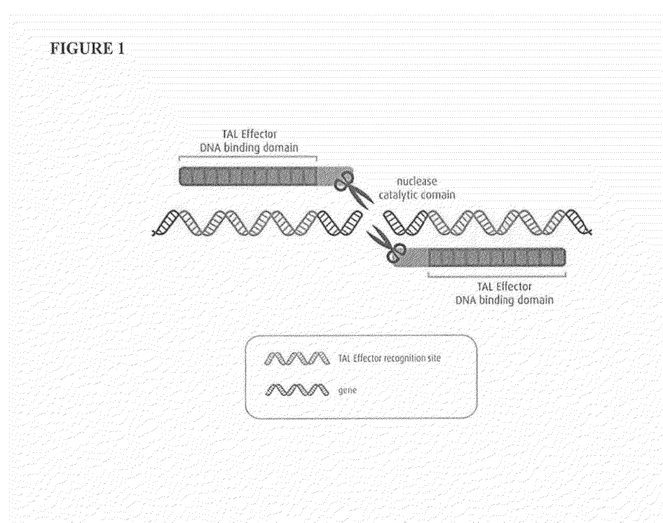

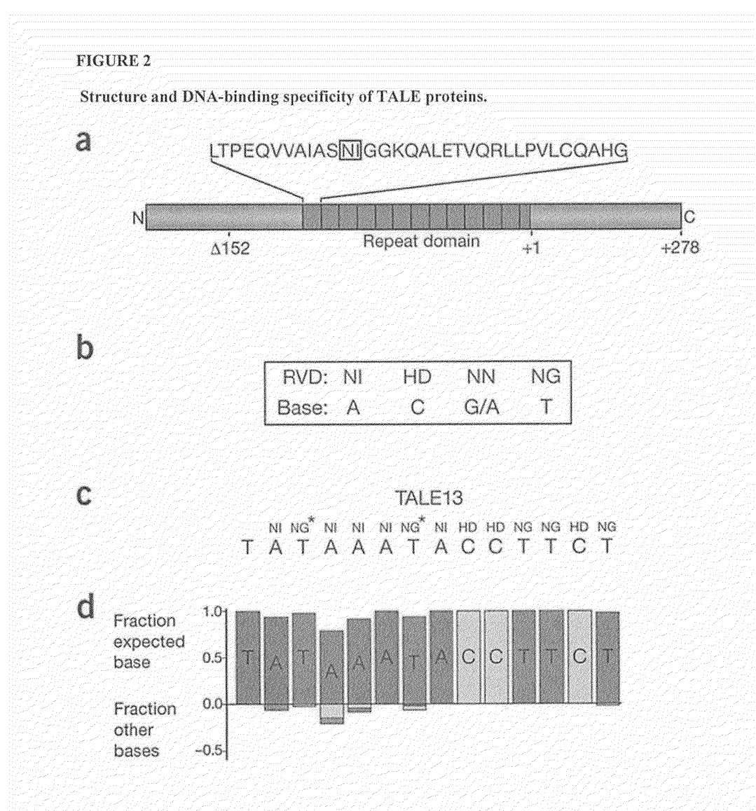

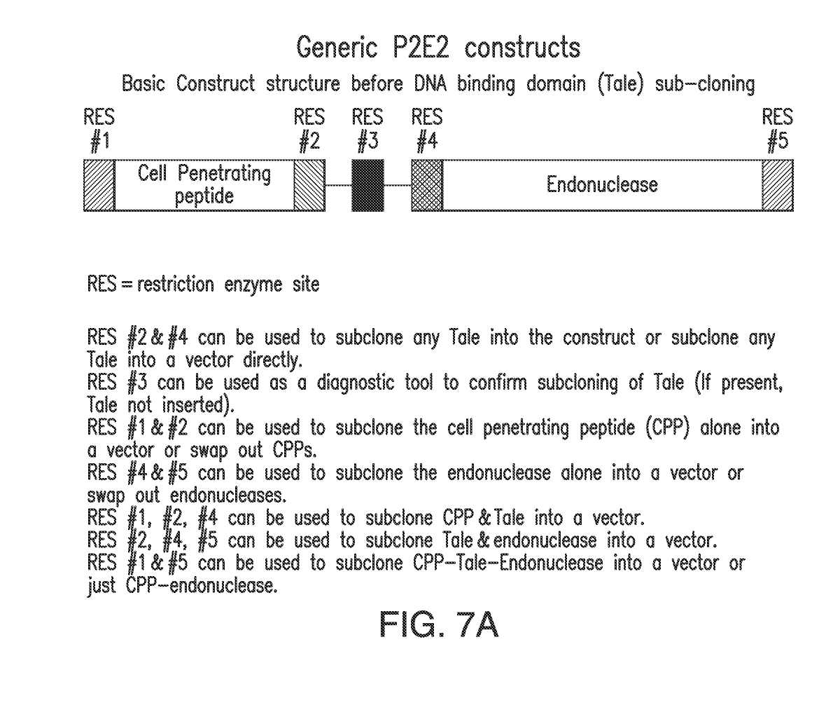

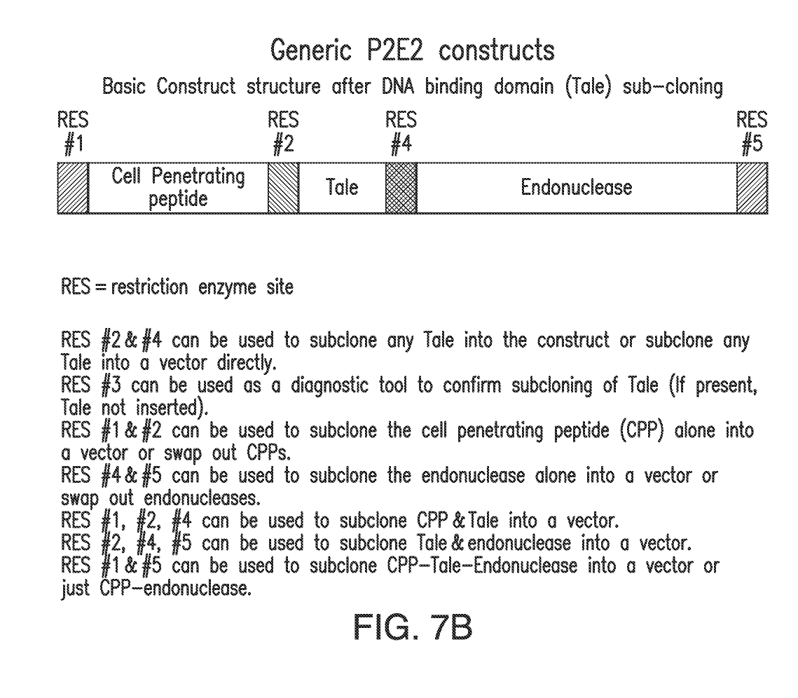

Obtain the cell penetrating peptide (CPP) DNA, two possible sources include from a vector or through chemical synthesis (e.g., G-block). Obtain the endonuclease DNA, again two possible sources include from a vector or as a G-block. In the example provided in FIG. 1A, the CPP and G-block have been synthesized using Gibson Assembly of G-blocks. Restriction enzyme sites (RESs) 2, 3, & 4 are included in this construct to allow flexibility and confirmation in TALEN subcloning. RESs 2 and 4 allow for subcloning and swapping in/out of DNA binding domains (TALEs) of interest (FIG. 1B). Restriction site 3 allows for verification of the presence of the subcloned DNA binding domain (if present, cloning failed). RESs 1 and 5 are initially designated in the G-block design but once the CPP-endonuclease DNA is built, these can be changed by PCR using forward (RES #1) and reverse (RES #5) primers for subcloning different REs in a variety of vector backbones. This is shown in FIG. 7.

### Generic Construct Testing

P2E2 constructs can be tested both in vitro and in vivo for their abilities to bind and cut DNA specifically. In vitro, the 3-part protein can be expressed, purified and tested for binding to target DNA using a variety of methods including EMSA, South-western blotting, and pull-down assays. To test cutting of target DNA, PCR & sequencing can be employed to verify deletions and/or insertions. To test specificity of both binding and cutting of the 3-part protein, base pairs in the target DNA can be mutated and binding & cutting assays performed.

In vivo, the P2E2 construct can be tested in either its DNA form (transfected in) or in its protein form. If using the protein form, the cell penetrating capability and localization of the P2E2 construct protein can be assessed using a variety of methods including staining techniques and western blotting. Binding of the P2E2 construct to the target DNA, and subsequent cleavage can be assessed using similar techniques discussed previously.

### Prophetic Example for Targeting HIV Genome Excision

In this example, 4 pairs of P2E2 constructs are built to target a specific sequence in HIV-1 B subtype proviral DNA, the TAR region (Table 4). This region is highly conserved in HIV-1 B subtype viruses and is important for viral replication. The TAR region is repeated with two copies, one near the beginning and one near the end of the HIV genome. This will target the flanked HIV genome for deletion by the three component P2E2 constructs.

TABLE 4
Targeted HIV proviral DNA region.

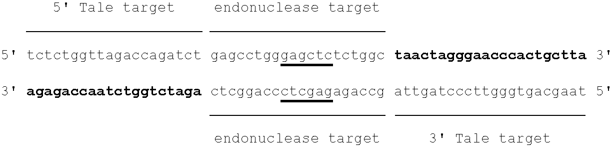

The first twenty nucleobases (t, c, g and a) in 5′ and the last twenty nucleobases in 3′ are the potential DNA binding target nucleotides for a TALE. The central twenty nucleobases in each is the potential region for nuclease activity, dependent on the endonuclease.

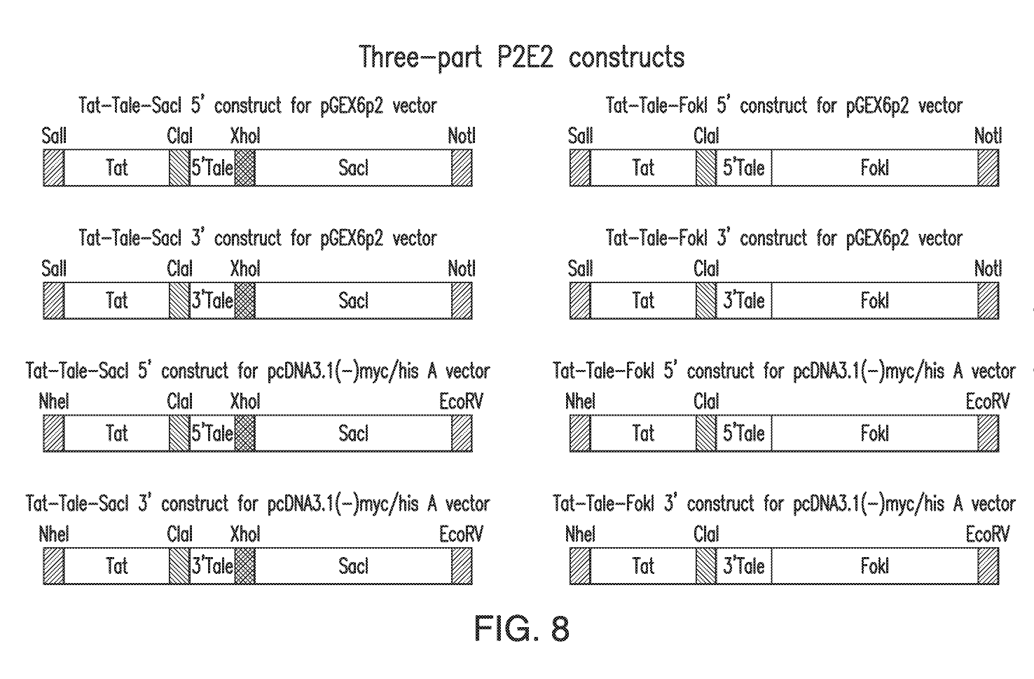

The 5′ TALE constructs will target “tctctggttagaccagatct” for binding while the 3′ Tale constructs will target “taagcagtgggttccctagtta” for binding. The pairs of P2E2 constructs containing the FokI catalytic core will target within the “gagcctgggagctctctggc” of the red region for cutting while those P2E2 constructs containing SacI will specifically target the “gagctc” sequence within the red region. The P2E2 constructs will consist of a cell penetrating peptide component (Tat), a DNA binding domain component (either 5′ or 3′ Tale), and an endonuclease component (SacI or FokI) (See FIG. 8). Restriction enzyme sites at the 5′ and 3′ ends of the P2E2 construct will vary depending on which vector the P2E2 construct is cloned into, pGEX6P2 for expression in E. coli and purification of the three component protein or pcDNA3.1(−)myc/his A for expression in mammalian cells.

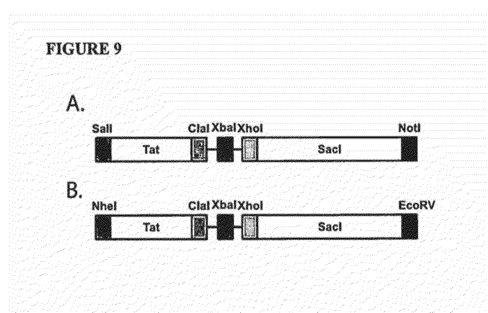

To build the P2E2 constructs of FIG. 8, various pieces are assembled in a step-wise manner.
1. Prepare the vectors. Both the pGEX6P2 and pcDNA3.1(−)myc/his A vectors must be prepared to receive DNA. The pGEX6P2 vector is double-digested with the SalI and NotI restriction enzymes, followed by treatment with Antarctic phosphatase. The pcDNA3.1(−)myc/his A vector is double-digested with the NheI and EcoRV restriction enzymes, followed by treatment with Antarctic phosphatase.
2. Prepare and ligate the Tat-SacI insert into the designated vectors. We will initially build the following constructs shown in FIG. 9 using Gibson assembly of G-blocks and PCR:

Construct A DNA of FIG. 9 will be double-digested with Sail and NotI to be eventually ligated into pGEX6P2. Construct B DNA of FIG. 9 will be double-digested with NheI and EcoRV to be eventually ligated into pcDNA3.1(−)myc/his A. The G-block sequences are provided below.

### Gblocks TAT and SacI

### Gblock1: 303 Nucleotides, NheI Site, Kozak Sequence, HIV-1 TAT Protein ClaI Site, XbaI Site, XhoI Site

GCTAGCGCCGCCACCATGGAGCCAGTAGATCCTAGACTAGAGCCCTGGA
AGCATCCAGGAAGTCAGCCTAAAACTGCTTGTACCAATTGCTATTGTAA
AAAGTGTTGCTTTCATTGCCAAGTTTGTTTCATAACAAAAGCCTTAGGC
ATCTCCTATGGCAGGAAGAAGCGGAGACAGCGACGAAGAGCACATCAGA
ACAGTCAGACTCATCAAGCTTCTCTATCAAAGCAACCCACCTCCCAACC
CCGAGGGGACCCGACAGGCCCGAAGGAAATCGATGCTGGTTCTAGAGCA
GGACTCGAG

### Gblock2: 370 Nucleotides, ClaI Site, XbaI Site, XhoI Site, Beginning of SacI Endonuclease Protein

ATCGATGCTGGTTCTAGAGCAGGACTCGAGATGGGCATAACGATAAAAA
AGAGCACTGCCGAGCAGGTTCTAAGGAAAGCCTACGAAGCTGCGGCATC
AGACGATGTGTTTCTTGAGGACTGGATCTTTCTGGCTACTAGTCTCCGC
GAGGTCGATGCCCCTAGAACATACACCGCCGCGCTAGTCACCGCTTTGC
TTGCACGTGCTTGTGACGATCGAGTTGATCCCAGGAGCATTAAAGAAAA
ATATGACGATAGAGCGTTTTCCTTAAGGACGCTTTGTCATGGGGTAGTT
GTACCAATGTCGGTTGAGTTAGGATTTGACCTTGGCGCGACAGGAAGA
GAGCCTATAAACAATCAGCCCTTTTTTC

### Gblock3: 400 Nucleotides, SacI Endonuclease Protein

GAGAGCCTATAAACAATCAGCCCTTTTTTCGCTACGATCAGTACAGTGA
AATCGTCAGGGTCCAAACCAAAGCCAGACCGTATTTGGACCGAGTATCG
TCAGCCTTAGCCAGGGTCGATGAAGAGGATTATTCGACGGAAGAGAGCT
TTCGCGCATTAGTAGCGGTGCTCGCGGTTTGTATCAGTGTTGCGAACAA
GAAACAAAGAGTTGCAGTAGGGTCAGCTATTGTTGAAGCAAGTTTAATC
GCAGAGACACAAAGCTTTGTGGTAAGCGGCCACGACGTTCCTCGGAAGT
TGCAAGCCTGTGTGGCAGCCGGATTGGACATGGTATATAGTGAAGTCGT
ATCGCGACGTATAAATGACCCGTCCCGTGATTTTCCTGGGGATGTTCAG
GTAATCTT

### Gblock4: 400 Nucleotides, End of SacI Endonuclease Protein, EcoRV Site

TGATTTTCCTGGGGATGTTCAGGTAATCTTAGATGGAGACCCATTGC
TGACAGTCGAGGTACGTGGTAAGTCTGTGAGCTGGGAGGGTCTCGAA
CAATTTGTGTCTTCAGCAACGTACGCGGGTTTTAGGCGCGTGGCACT
AATGGTGGATGCGGCTTCCCACGTGTCACTGATGTCTGCTGATGACC
TAACTTCAGCTTTGGAGCGGAAATATGAGTGTATTGTCAAGGTAAAT
GAGAGCGTCAGTTCCTTTCTCCGAGACGTATTTGTCTGGTCTCCAAG
GGATGTGCATAGTATTCTATCAGCTTTTCCCGAAGCAATGTATAGAC
GGATGATTGAAATAGAAGTACGGGAACCGGAACTGGACAGATGGGCT
GAGATATTTCCAGAAACTGATATC

3. Preparing the Tat-SacI vectors. Once the vectors contain the Tat-SacI inserts, they will be double digested with ClaI and XhoI and then treated with Antarctic phosphatase to prepare them for the TALEN subcloning step.
4. Assembly of Tale monomers using the Real Assembly kit. TALEs are constructed from monomer plasmids using the Real Assembly kit. Examples of the assembly of the 5′ TALE and 3′ TALE are illustrated on the next page. Sequences of each monomer are included following the 5′/3′Tale illustration.
5. Quick change mutagenesis will be performed on select monomer plasmids in order to obtain a monomer containing the “NS” di-residue, that will recognize any nucleotide. This is for the purpose of target sequence positions that do not have 100% conservation in the HIV subtype B virus sequences.
6. Once the monomers (approximately 18.5 and 20.5) are compiled and ligated into the Real Assembly kit plasmids, PCR will be performed to produce cDNA of the TALE and TALE-FokI insert (FokI obtained from the Real Assembly kit plasmid) with the correct flanking restriction enzyme sites for insertion into the vectors. These cDNAs will be double digested with their designated enzymes (ClaI/XhoI for the Tale, ClaI/EcoRV or ClaI/NotI for the TALE-FokI) and then ligated into their designated vectors. The final construct DNA and amino acid sequences can be found under “Final DNA & amino acid sequences” provided below.

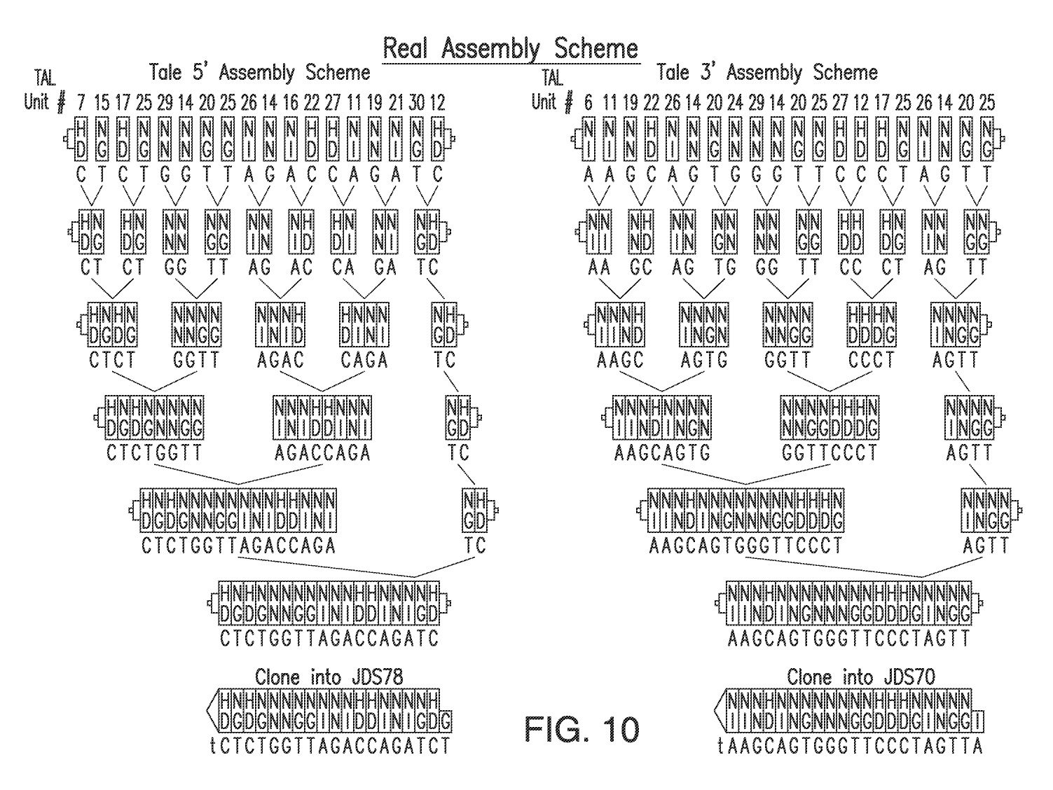

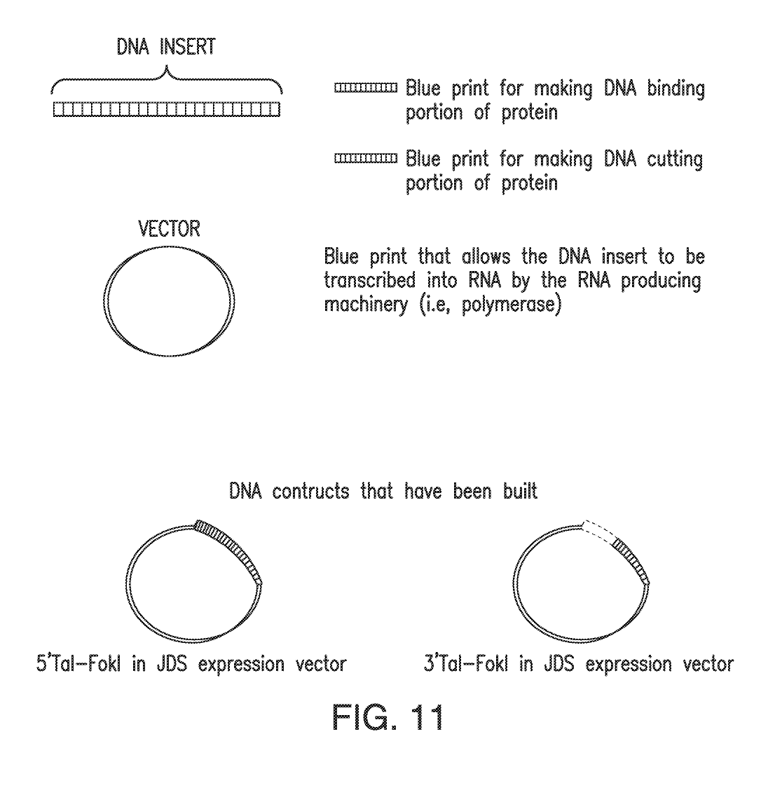

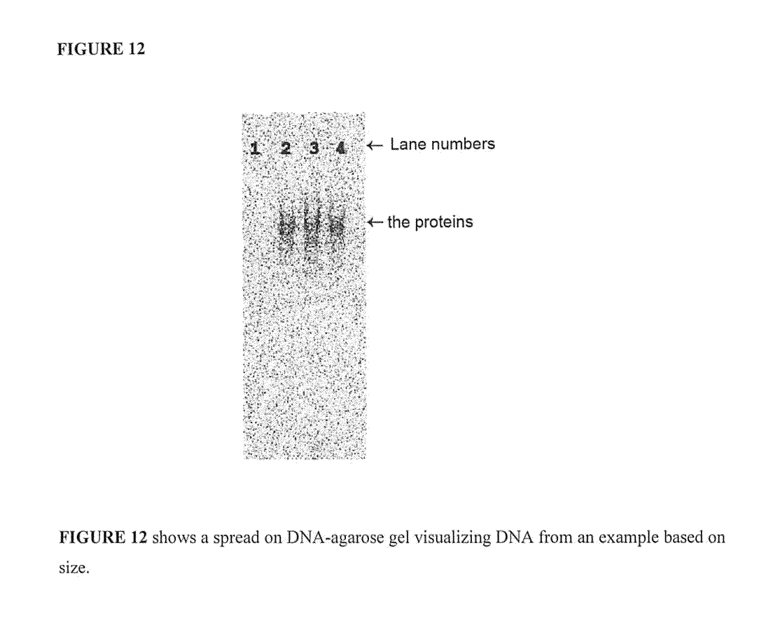

The actual assembly sequence of steps is shown in FIG. 10.

### TALE Plasmid Sequences

### TAL 007: C Binder

DNA:
GAACCTGACCCCAGACCAGGTAGTCGCAATCGCGTCACATGA
CGGGGGAAAGCAAGCCCTGGAAACCGTGCAAAGGTTGTTGCCGGTCCTT
TGTCAAGACCACGGC
Protein:
N L T P D Q V V A I A S H D G G K Q A L E T V Q R
L L P V L C Q D H G

### TAL 015: T Binder

DNA:
CTTACACCGGAGCAAGTCGTGGCCATTGCAAGCAATGGGGGT
GGCAAACAGGCTCTTGAGACGGTTCAGAGACTTCTCCCAGTT
CTCTGTCAAGCCCACGGG
Protein:
L T P E Q V V A I A S N G G G K Q A L E T V
Q R L L P V L C Q A H G

### TAL 017: C Binder

DNA:
CTGACTCCCGATCAAGTTGTAGCGATTGCGTCGCATGACGGA
GGGAAACAAGCATTGGAGACTGTCCAACGGCTCCTTCCCGTG
TTGTGTCAAGCCCACGGT
Protein:
L T P D Q V V A I A S H D G G K Q A L E T V
Q R L L P V L C Q A H G

### TAL 025: T Binder

DNA:
TTGACGCCTGCACAAGTGGTCGCCATCGCCTCGAATGGCGGC
GGTAAGCAGGCGCTGGAAACAGTACAGCGCCTGCTGCCTGTA
CTGTGCCAGGATCATGGA
Protein:
L T P A Q V V A I A S N G G G K Q A L E T V
Q R L L P V L C Q D H G

### NS Mutant:

DNA:
TTGACGCCTGCACAAGTGGTCGCCATCGCCTCGAATTCGGGC
GGTAAGCAGGCGCTGGAAACAGTACAGCGCCTGCTGCCTGTA
CTGTGCCAGGATCATGGA
Protein:
L T P A Q V V A I A S N S G G K Q A L E T V
Q R L L P V L C Q D H G

### TAL 029: G Binder

DNA:
CTGACCCCAGACCAGGTAGTCGCAATCGCGAACAATAATGGG
GGAAAGCAAGCCCTGGAAACCGTGCAAAGGTTGTTGCCGGTC
CTTTGTCAAGACCACGGC
Protein:
L T P D Q V V A I A N N N G G K Q A L E T V
Q R L L P V L C Q D H G

### NS Mutant: AATTCG/N S

DNA:
CTGACCCCAGACCAGGTAGTCGCAATCGCGAACAATTCGGGG
GGAAAGCAAGCCCTGGAAACCGTGCAAAGGTTGTTGCCGGTC
CTTTGTCAAGACCACGGC
Protein:
L T P D Q V V A I A N N S G G K Q A L E T V
Q R L L P V L C Q D H G

### TAL 014: G Binder

DNA:
CTTACACCGGAGCAAGTCGTGGCCATTGCAAATAATAACGGT
GGCAAACAGGCTCTTGAGACGGTTCAGAGACTTCTCCCAGTT
CTCTGTCAAGCCCACGGG
Protein:
L T P E Q V V A I A N N N G G K Q A L E T V
Q R L L P V L C Q A H G

### TAL 020: T Binder

DNA:
CTGACTCCCGATCAAGTTGTAGCGATTGCGTCCAACGGTGGA
GGGAAACAAGCATTGGAGACTGTCCAACGGCTCCTTCCCGTG
TTGTGTCAAGCCCACGGT
Protein:
L T P D Q V V A I A S N G G G K Q A L E T V
Q R L L P V L C Q A H G

### TAL 026: A Binder

DNA:
CTGACCCCAGACCAGGTAGTCGCAATCGCGTCGAACATTGGG
GGAAAGCAAGCCCTGGAAACCGTGCAAAGGTTGTTGCCGGTC
CTTTGTCAAGACCACGGC
Protein:
L T P D Q V V A I A S N I G G K Q A L E T V
Q R L L P V L C Q D H G

### TAL 016: A Binder

DNA:
CTGACTCCCGATCAAGTTGTAGCGATTGCGTCGAACATTGGA
GGGAAACAAGCATTGGAGACTGTCCAACGGCTCCTTCCCGTG
TTGTGTCAAGCCCACGGT
Protein:
L T P D Q V V A I A S N I G G K Q A L E T V
Q R L L P V L C Q A H G

### TAL 022: C Binder

DNA:
TTGACGCCTGCACAAGTGGTCGCCATCGCCAGCCATGATGGC
GGTAAGCAGGCGCTGGAAACAGTACAGCGCCTGCTGCCTGTA
CTGTGCCAGGATCATGGA
Protein:
L T P A Q V V A I A S H D G G K Q A L E T V
Q R L L P V L C Q D H G

### TAL 027: C Binder

DNA:
CTGACCCCAGACCAGGTAGTCGCAATCGCGTCACATGACGGG
GGAAAGCAAGCCCTGGAAACCGTGCAAAGGTTGTTGCCGGTC
CTTTGTCAAGACCACGGC
Protein:
L T P D Q V V A I A S H D G G K Q A L E T V
Q R L L P V L C Q D H G

### TAL 011: A Binder

DNA:
CTTACACCGGAGCAAGTCGTGGCCATTGCAAGCAACATCGGT
GGCAAACAGGCTCTTGAGACGGTTCAGAGACTTCTCCCAGTT
CTCTGTCAAGCCCACGGG
Protein:
L T P E Q V V A I A S N I G G K Q A L E T V
Q R L L P V L C Q A H G

### TAL 019: G Binder

DNA:
CTGACTCCCGATCAAGTTGTAGCGATTGCGAATAACAATGGA
GGGAAACAAGCATTGGAGACTGTCCAACGGCTCCTTCCCGTG
TTGTGTCAAGCCCACGGT
Protein:
L T P D Q V V A I A N N N G G K Q A L E T V
Q R L L P V L C Q A H G

### TAL 021: A Binder

DNA:
TTGACGCCTGCACAAGTGGTCGCCATCGCCTCCAATATTGGCG
GTAAGCAGGCGCTGGAAACAGTACAGCGCCTGCTGCCTGTACT
GTGCCAGGATCATGGA
Protein:
L T P A Q V V A I A S N I G G K Q A L E T V
Q R L L P V L C Q D H G

### TAL 030: T Binder

DNA:
CTGACCCCAGACCAGGTAGTCGCAATCGCGTCAAACGGAGGGG
GAAAGCAAGCCCTGGAAACCGTGCAAAGGTTGTTGCCGGTCCT
TTGTCAAGACCACGGC
Protein:
L T P D Q V V A I A S N G G G K Q A L E T V
Q R L L P V L C Q D H G

### TAL 012: C Binder

DNA:
CTTACACCGGAGCAAGTCGTGGCCATTGCATCCCACGACGGTG
GCAAACAGGCTCTTGAGACGGTTCAGAGACTTCTCCCAGTTCT
CTGTCAAGCCCACGGG
Protein:
L T P E Q V V A I A S H D G G K Q A L E T V
Q R L L P V L C Q A H G

### NS Mutant:

AATTCG/N S
DNA:
CTTACACCGGAGCAAGTCGTGGCCATTGCATCCAATTCGGGTGGCAAAC
AGGCTCTTGAGACGGTTCAGAGACTTCTCCCAGTTCTCTGTCAAGCCCA
CGGG
Protein:
L T P E Q V V A I A S N S G G K Q A L E T V Q R L
L P V L C Q A H G

### TAL 006: A Binder

DNA:
GAACCTGACCCCAGACCAGGTAGTCGCAATCGCGTCGAACATT
GGGGGAAAGCAAGCCCTGGAAACCGTGCAAAGGTTGTTGCCGG
TCCTTTGTCAAGACCACGGC
Protein:
N L T P D Q V V A I A S N I G G K Q A L E T
V Q R L L P V L C Q D H G

### TAL 024: G Binder

DNA:
TTGACGCCTGCACAAGTGGTCGCCATCGCCAACAACAACGGCG
GTAAGCAGGCGCTGGAAACAGTACAGCGCCTGCTGCCTGTACT
GTGCCAGGATCATGGA
Protein:
L T P A Q V V A I A N N N G G K Q A L E T V
Q R L L P V L C Q D H G

### TAL 012: C Binder

DNA:
CTTACACCGGAGCAAGTCGTGGCCATTGCATCCCACGACGGTG
GCAAACAGGCTCTTGAGACGGTTCAGAGACTTCTCCCAGTTCT
CTGTCAAGCCCACGGG
Protein:
L T P E Q V V A I A S H D G G K Q A L E T V
Q R L L P V L C Q A H G

### TAL 017: C Binder

DNA:
CTGACTCCCGATCAAGTTGTAGCGATTGCGTCGCATGACGGAG
GGAAACAAGCATTGGAGACTGTCCAACGGCTCCTTCCCGTGTT
GTGTCAAGCCCACGGT
Protein:
L T P D Q V V A I A S H D G G K Q A L E T V
Q R L L P V L C Q A H G

### Final DNA & Amino Acid Sequences

### TAT-TALE-FOKI (Forward 5′):

### DNA: BOLD Capital=TAT

### Capital ITALICS=TALE

Underlined capitals are Nuclease
Capitals, neither BOLD, Italicized nor Underlined are not TAT, TALE or Nuclease (FokI) sequences

GCTAGCGCCGCCACCGATGGAGCCAGTAGATCCTAGACTAGAG
CCCTGGAAGCATCCAGGAAGTCAGCCTAAAACTGCTTGTACCA
ATTGCTATTGTAAAAAGTGTTGCTTTCATTGCCAAGTTTGTTT
CATAACAAAAGCCTTAGGCATCTCCtatggcaggaagaagcgg
agacagcgacgaagaGCAGgaGCACATCAGAACAGTCAGACTC
ATCAAGCTTCTCTATCAAAGCAACCCACCTCCCAACCCCGAGG
GGACCCGACAGGCCCGAAGGAAATCGATAAGAAGAAGAGGAAG
GTGGGCATTCACCGCGGGGTACCTATGGTGGACTTGAGGACAC
TCGGTTATTCGCAACAGCAACAGGAGAAAATCAAGCCTAAGGT
CAGGAGCACCGTCGCGCAACACCACGAGGCGCTTGTGGGGCAT
GGCTTCACTCATGCGCATATTGTCGCGCTTTCACAGCACCCTG
CGGCGCTTGGGACGGTGGCTGTCAAATACCAAGATATGATTGC
GGCCCTGCCCGAAGCCACGCACGAGGCAATTGTAGGGGTCGGT
AAACAGTGGTCGGGAGCGCGAGCACTTGAGGCGCTGCTGACTG
TGGCGGGTGAGCTTAGGGGGCCTCCGCTCCAGCTCGACACCGG
GCAGCTGCTGAAGATCGCGAAGAGAGGGGGAGTAACAGCGGTA
GAGGCAGTGCACGCCTGGCGCAATGCGCTCACCGGGGCCCCCT
TGAACCTGACCCCAGACCAGGTAGTCGCAATCGCGTCACATGA
CGGGGGAAAGCAAGCCCTGGAAACCGTGCAAAGGTTGTTGCCG
GTCCTTTGTCAAGACCACGGCCTTACACCGGAGCAAGTCGTGG
CCATTGCAAGCAATGGGGGTGGCAAACAGGCTCTTGAGACGGT
TCAGAGACTTCTCCCAGTTCTCTGTCAAGCCCACGGGCTGACT
CCCGATCAAGTTGTAGCGATTGCGTCGCATGACGGAGGGAAAC
AAGCATTGGAGACTGTCCAACGGCTCCTTCCCGTGTTGTGTCA
AGCCCACGGTTTGACGCCTGCACAAGTGGTCGCCATCGCCTCG
AATGGCGGCGGTAAGCAGGCGCTGGAAACAGTACAGCGCCTGC
TGCCTGTACTGTGCCAGGATCATGGACTGACCCCAGACCAGGT
AGTCGCAATCGCGAACAATTCGGGGGGAAAGCAAGCCCTGGAA
ACCGTGCAAAGGTTGTTGCCGGTCCTTTGTCAAGACCACGGCC
TTACACCGGAGCAAGTCGTGGCCATTGCAAATAATAACGGTGG
CAAACAGGCTCTTGAGACGGTTCAGAGACTTCTCCCAGTTCTC
TGTCAAGCCCACGGGCTGACTCCCGATCAAGTTGTAGCGATTG
CGTCCAACGGTGGAGGGAAACAAGCATTGGAGACTGTCCAACG
GCTCCTTCCCGTGTTGTGTCAAGCCCACGGTTTGACGCCTGCA
CAAGTGGTCGCCATCGCCTCGAATTCGGGCGGTAAGCAGGCGC
TGGAAACAGTACAGCGCCTGCTGCCTGTACTGTGCCAGGATCA
TGGACTGACCCCAGACCAGGTAGTCGCAATCGCGAACAATAAT
GGGGGAAAGCAAGCCCTGGAAACCGTGCAAAGGTTGTTGCCGG
TCCTTTGTCAAGACCACGGCCTTACACCGGAGCAAGTCGTGGC
CATTGCAAATAATAACGGTGGCAAACAGGCTCTTGAGACGGTT
CAGAGACTTCTCCCAGTTCTCTGTCAAGCCCACGGGCTGACTC
CCGATCAAGTTGTAGCGATTGCGTCGAACATTGGAGGGAAACA
AGCATTGGAGACTGTCCAACGGCTCCTTCCCGTGTTGTGTCAA
GCCCACGGTTTGACGCCTGCACAAGTGGTCGCCATCGCCAGCC
ATGATGGCGGTAAGCAGGCGCTGGAAACAGTACAGCGCCTGCT
GCCTGTACTGTGCCAGGATCATGGACTGACCCCAGACCAGGTA
GTCGCAATCGCGTCACATGACGGGGGAAAGCAAGCCCTGGAAA
CCGTGCAAAGGTTGTTGCCGGTCCTTTGTCAAGACCACGGCCT
TACACCGGAGCAAGTCGTGGCCATTGCAAGCAACATCGGTGGC
AAACAGGCTCTTGAGACGGTTCAGAGACTTCTCCCAGTTCTCT
GTCAAGCCCACGGGCTGACTCCCGATCAAGTTGTAGCGATTGC
GAATAACAATGGAGGGAAACAAGCATTGGAGACTGTCCAACGG
CTCCTTCCCGTGTTGTGTCAAGCCCACGGTTTGACGCCTGCAC
AAGTGGTCGCCATCGCCTCCAATATTGGCGGTAAGCAGGCGCT
GGAAACAGTACAGCGCCTGCTGCCTGTACTGTGCCAGGATCAT
GGACTGACCCCAGACCAGGTAGTCGCAATCGCGTCAAACGGAG
GGGGAAAGCAAGCCCTGGAAACCGTGCAAAGGTTGTTGCCGGT
CCTTTGTCAAGACCACGGCCTTACACCGGAGCAAGTCGTGGCC
ATTGCATCCAATTCGGGTGGCAAACAGGCTCTTGAGACGGTTC
AGAGACTTCTCCCAGTTCTCTGTCAAGCCCACGGGCTGACACC
CGAACAGGTGGTCGCCATTGCTTCTAATGGGGGAGGACGGCCA
GCCTTGGAGTCCATCGTAGCCCAATTGTCCAGGCCCGATCCCG
CGTTGGCTGCGTTAACGAATGACCATCTGGTGGCGTTGGCATG
TCTTGGTGGACGACCCGCGCTCGATGCAGTCAAAAAGGGTCTG
CCTCATGCTCCCGCATTGATCAAAAGAACCAACCGGCGGATTC
CCGAGAGAACTTCCCATCGAGTCGCGGGATCCCAACTAGTCAA
AAGTGAACTGGAGGAGAAGAAATCTGAACTTCGTCATAAATTG
AAATATGTGCCTCATGAATATATTGAATTAATTGAAATTGCCA
GAAATTCCACTCAGGATAGAATTCTTGAAATGAAGGTAATGGA
ATTTTTTATGAAAGTTTATGGATATAGAGGTAAACATTTGGGT
GGATCAAGGAAACCGGACGGAGCAATTTATACTGTCGGATCTC
CTATTGATTACGGTGTGATCGTGGATACTAAAGCTTATAGCGG
AGGTTATAATCTGCCAATTGGCCAAGCAGATGAAATGCAACGA
TATGTCGAAGAAAATCAAACACGAAACAAACATATCAACCCTA
ATGAATGGTGGAAAGTCTATCCATCTTCTGTAACGGAATTTAA
GTTTTTATTTGTGAGTGGTCACTTTAAAGGAAACTACAAAGCT
CAGCTTACACGATTAAATCATATCACTAATTGTAATGGAGCTG
TTCTTAGTGTAGAAGAGCTTTTAATTGGTGGAGAAATGATTAA
AGCCGGCACATTAACCTTAGAGGAAGTCAGACGGAAATTTAAT
AACGGCGAGATAAACTTTGATATC

### Protein:

### BOLD Capital=TAT

### Capital ITALICS=TALE

### UNDERLINED Capitals=Nuclease (FokI)

### 5′3′ Frame 1

A S A A T Met E P V D P R L E P W K H P G S
Q P K T A C T N C Y C K K C C F H C Q V C F
I T K A L G I S Y G R K K R R Q R R R A H Q
N S Q T H Q A S L S K Q P T S Q P R G D P T
G P K E I D K K K R K V G I H R G V P [symbol] V
D L R T L G Y S Q Q Q Q E K I K P K V R S T
V A Q H H E A L V G H G F T H A H I V A L S
Q H P A A L G T V A V K Y Q D [symbol] I A A L P
E A T H E A I V G V G K Q W S G A R A L E A
L L T V A G E L R G P P L Q L D T G Q L L K
I A K R G G V T A V E A V H A W R N A L T G
A P L N L T P D Q V V A I A S H D G G K Q A
L E T V Q R L L P V L C Q D H G L T P E Q V
V A I A S N G G G K Q A L E T V Q R L L P V
L C Q A H G L T P D Q V V A I A S H D G G K
Q A L E T V Q R L L P V L C Q A H G L T P A
Q V V A I A S N G G G K Q A L E T V Q R L L
P V L C Q D H G L T P D Q V V A I A N N S G
G K Q A L E T V Q R L L P V L C Q D H G L T
P E Q V V A I A N N N G G K Q A L E T V Q R
L L P V L C Q A H G L T P D Q V V A I A S N
G G G K Q A L E T V Q R L L P V L C Q A H G
L T P A Q V V A I A S N S G G K Q A L E T V
Q R L L P V L C Q D H G L T P D Q V V A I A
N N N G G K Q A L E T V Q R L L P V L C Q D
H G L T P E Q V V A I A N N N G G K Q A L E
T V Q R L L P V L C Q A H G L T P D Q V V A
I A S N I G G K Q A L E T V Q R L L P V L C
Q A H G L T P A Q V V A I A S H D G G K Q A
L E T V Q R L L P V L C Q D H G L T P D Q V
V A I A S H D G G K Q A L E T V Q R L L P V
L C Q D H G L T P E Q V V A I A S N I G G K
Q A L E T V Q R L L P V L C Q A H G L T P D
Q V V A I A N N N G G K Q A L E T V Q R L L
P V L C Q A H G L T P A Q V V A I A S N I G
G K Q A L E T V Q R L L P V L C Q D H G L T
P D Q V V A I A S N G G G K Q A L E T V Q R
L L P V L C Q D H G L T P E Q V V A I A S N
S G G K Q A L E T V Q R L L P V L C Q A H G
L T P E Q V V A I A S N G G G R P A L E S I
V A Q L S R P D P A L A A L T N D H L V A L
A C L G G R P A L D A V K K G L P H A P A L
I K R T N R R I P E R T S H R V A G S Q L V
K S E L E E K K S E L R H K L K Y V P H E Y
I E L I E I A R N S T Q D R I L E Met K V
Met E F F Met K V Y G Y R G K H L G G S R K
P D G A I Y T V G S P I D Y G V I V D T K A
Y S G G Y N L P I G Q A D E Met Q R Y V E E
N Q T R N K H I N P N E W W K V Y P S S V T
E F K F L F V S G H F K G N Y K A Q L T R L
N H I T N C N G A V L S V E E L L I G G E
Met I K A G T L T L E E V R R K F N N G E I
N F D I

### TAT-TALE-FOKI (Reverse 3′):

DNA: BOLD Capital=TAT

Capital ITALICS=TALE

UNDERLINED Capitals=Nuclease (FokI)

GCTAGCGCCGCCACCATGGAGCCAGTAGATCCTAGACTAGAGC
CCTGGAAGCATCCAGGAAGTCAGCCTAAAACTGCTTGTACCAA
TTGCTATTGTAAAAAGTGTTGCTTTCATTGCCAAGTTTGTTTC
ATAACAAAAGCCTTAGGCATCTCCtatggcaggaagaagcgga
gacagcgacgaagaGCACATCAGAACAGTCAGACTCATCAAGC
TTCTCTATCAAAGCAACCCACCTCCCAACCCCGAGGGGACCCG
ACAGGCCCGAAGGAAATCGATAAGAAGAAGAGGAAGGTGGGCA
TTCACCGCGGGGTACCTATGGTGGACTTGAGGACACTCGGTTA
TTCGCAACAGCAACAGGAGAAAATCAAGCCTAAGGTCAGGAGC
ACCGTCGCGCAACACCACGAGGCGCTTGTGGGGCATGGCTTCA
CTCATGCGCATATTGTCGCGCTTTCACAGCACCCTGCGGCGCT
TGGGACGGTGGCTGTCAAATACCAAGATATGATTGCGGCCCTG
CCCGAAGCCACGCACGAGGCAATTGTAGGGGTCGGTAAACAGT
GGTCGGGAGCGCGAGCACTTGAGGCGCTGCTGACTGTGGCGGG
TGAGCTTAGGGGGCCTCCGCTCCAGCTCGACACCGGGCAGCTG
CTGAAGATCGCGAAGAGAGGGGGAGTAACAGCGGTAGAGGCAG
TGCACGCCTGGCGCAATGCGCTCACCGGGGCCCCCTTGAACCT
GACCCCAGACCAGGTAGTCGCAATCGCGTCGAACATTGGGGGA
AAGCAAGCCCTGGAAACCGTGCAAAGGTTGTTGCCGGTCCTTT
GTCAAGACCACGGCCTTACACCGGAGCAAGTCGTGGCCATTGC
AAGCAACATCGGTGGCAAACAGGCTCTTGAGACGGTTCAGAGA
CTTCTCCCAGTTCTCTGTCAAGCCCACGGGCTGACTCCCGATC
AAGTTGTAGCGATTGCGAATAACAATGGAGGGAAACAAGCATT
GGAGACTGTCCAACGGCTCCTTCCCGTGTTGTGTCAAGCCCAC
GGTTTGACGCCTGCACAAGTGGTCGCCATCGCCAGCCATGATG
GCGGTAAGCAGGCGCTGGAAACAGTACAGCGCCTGCTGCCTGT
ACTGTGCCAGGATCATGGACTGACCCCAGACCAGGTAGTCGCA
ATCGCGTCGAACATTGGGGGAAAGCAAGCCCTGGAAACCGTGC
AAAGGTTGTTGCCGGTCCTTTGTCAAGACCACGGCCTTACACC
GGAGCAAGTCGTGGCCATTGCAAATAATAACGGTGGCAAACAG
GCTCTTGAGACGGTTCAGAGACTTCTCCCAGTTCTCTGTCAAG
CCCACGGGCTGACTCCCGATCAAGTTGTAGCGATTGCGTCCAA
CGGTGGAGGGAAACAAGCATTGGAGACTGTCCAACGGCTCCTT
CCCGTGTTGTGTCAAGCCCACGGTTTGACGCCTGCACAAGTGG
TCGCCATCGCCAACAACAACGGCGGTAAGCAGGCGCTGGAAAC
AGTACAGCGCCTGCTGCCTGTACTGTGCCAGGATCATGGACTG
ACCCCAGACCAGGTAGTCGCAATCGCGAACAATAATGGGGGAA
AGCAAGCCCTGGAAACCGTGCAAAGGTTGTTGCCGGTCCTTTG
TCAAGACCACGGCCTTACACCGGAGCAAGTCGTGGCCATTGCA
AATAATAACGGTGGCAAACAGGCTCTTGAGACGGTTCAGAGAC
TTCTCCCAGTTCTCTGTCAAGCCCACGGGCTGACTCCCGATCA
AGTTGTAGCGATTGCGTCCAACGGTGGAGGGAAACAAGCATTG
GAGACTGTCCAACGGCTCCTTCCCGTGTTGTGTCAAGCCCACG
GTTTGACGCCTGCACAAGTGGTCGCCATCGCCTCGAATGGCGG
CGGTAAGCAGGCGCTGGAAACAGTACAGCGCCTGCTGCCTGTA
CTGTGCCAGGATCATGGACTGACCCCAGACCAGGTAGTCGCAA
TCGCGTCACATGACGGGGGAAAGCAAGCCCTGGAAACCGTGCA
AAGGTTGTTGCCGGTCCTTTGTCAAGACCACGGCCTTACACCG
GAGCAAGTCGTGGCCATTGCATCCCACGACGGTGGCAAACAGG
CTCTTGAGACGGTTCAGAGACTTCTCCCAGTTCTCTGTCAAGC
CCACGGGCTGACTCCCGATCAAGTTGTAGCGATTGCGTCGCAT
GACGGAGGGAAACAAGCATTGGAGACTGTCCAACGGCTCCTTC
CCGTGTTGTGTCAAGCCCACGGTTTGACGCCTGCACAAGTGGT
CGCCATCGCCTCGAATGGCGGCGGTAAGCAGGCGCTGGAAACA
GTACAGCGCCTGCTGCCTGTACTGTGCCAGGATCATGGACTGA
CCCCAGACCAGGTAGTCGCAATCGCGTCGAACATTGGGGGAAA
GCAAGCCCTGGAAACCGTGCAAAGGTTGTTGCCGGTCCTTTGT
CAAGACCACGGCCTTACACCGGAGCAAGTCGTGGCCATTGCAA
ATAATAACGGTGGCAAACAGGCTCTTGAGACGGTTCAGAGACT
TCTCCCAGTTCTCTGTCAAGCCCACGGGCTGACTCCCGATCAA
GTTGTAGCGATTGCGTCCAACGGTGGAGGGAAACAAGCATTGG
AGACTGTCCAACGGCTCCTTCCCGTGTTGTGTCAAGCCCACGG
TTTGACGCCTGCACAAGTGGTCGCCATCGCCTCGAATGGCGGC
GGTAAGCAGGCGCTGGAAACAGTACAGCGCCTGCTGCCTGTAC
TGTGCCAGGATCATGGACTGACACCCGAACAGGTGGTCGCCAT
TGCTTCTAACATCGGAGGACGGCCAGCCTTGGAGTCCATCGTA
GCCCAATTGTCCAGGCCCGATCCCGCGTTGGCTGCGTTAACGA
ATGACCATCTGGTGGCGTTGGCATGTCTTGGTGGACGACCCGC
GCTCGATGCAGTCAAAAAGGGTCTGCCTCATGCTCCCGCATTG
ATCAAAAGAACCAACCGGCGGATTCCCGAGAGAACTTCCCATC
GAGTCGCGGGATCCCAACTAGTCAAAAGTGAACTGGAGGAGAA
GAAATCTGAACTTCGTCATAAATTGAAATATGTGCCTCATGAA
TATATTGAATTAATTGAAATTGCCAGAAATTCCACTCAGGATA
GAATTCTTGAAATGAAGGTAATGGAATTTTTTATGAAAGTTTA
TGGATATAGAGGTAAACATTTGGGTGGATCAAGGAAACCGGAC
GGAGCAATTTATACTGTCGGATCTCCTATTGATTACGGTGTGA
TCGTGGATACTAAAGCTTATAGCGGAGGTTATAATCTGCCAAT
TGGCCAAGCAGATGAAATGCAACGATATGTCGAAGAAAATCAA
ACACGAAACAAACATATCAACCCTAATGAATGGTGGAAAGTCT
ATCCATCTTCTGTAACGGAATTTAAGTTTTTATTTGTGAGTGG
TCACTTTAAAGGAAACTACAAAGCTCAGCTTACACGATTAAAT
CATATCACTAATTGTAATGGAGCTGTTCTTAGTGTAGAAGAGC
TTTTAATTGGTGGAGAAATGATTAAAGCCGGCACATTAACCTT
AGAGGAAGTCAGACGGAAATTTAATAACGGCGAGATAAACTTT
GATATC

### Protein:

### BOLD CAPITALS=TAT

### Italicized=TALE

### Underlined=Endonuclease (FokI)

### 5′3′ Frame 1

A S A A T Met E P V D P R L E P W K H P G S
Q P K T A C T N C Y C K K C C F H C Q V C F
I T K A L G I S Y G R K K R R Q R R R A H Q
N S Q T H Q A S L S K Q P T S Q P R G D P T
G P K E I D K K K R K V G I H R G V P [symbol] V
D L R T L G Y S Q Q Q Q E K I K P K V R S T
V A Q H H E A L V G H G F T H A H I V A L S
Q H P A A L G T V A V K Y Q D [symbol] I A A L P
E A T H E A I V G V G K Q W S G A R A L E A
L L T V A G E L R G P P L Q L D T G Q L L K
I A K R G G V T A V E A V H A W R N A L T G
A P L N L T P D Q V V A I A S N I G G K Q A
L E T V Q R L L P V L C Q D H G L T P E Q V
V A I A S N I G G K Q A L E T V Q R L L P V
L C Q A H G L T P D Q V V A I A N N N G G K
Q A L E T V Q R L L P V L C Q A H G L T P A
Q V V A I A S H D G G K Q A L E T V Q R L L
P V L C Q D H G L T P D Q V V A I A S N I G
G K Q A L E T V Q R L L P V L C Q D H G L T
P E Q V V A I A N N N G G K Q A L E T V Q R
L L P V L C Q A H G L T P D Q V V A I A S N
G G G K Q A L E T V Q R L L P V L C Q A H G
L T P A Q V V A I A N N N G G K Q A L E T V
Q R L L P V L C Q D H G L T P D Q V V A I A
N N N G G K Q A L E T V Q R L L P V L C Q D
H G L T P E Q V V A I A N N N G G K Q A L E
T V Q R L L P V L C Q A H G L T P D Q V V A
I A S N G G G K Q A L E T V Q R L L P V L C
Q A H G L T P A Q V V A I A S N G G G K Q A
L E T V Q R L L P V L C Q D H G L T P D Q V
V A I A S H D G G K Q A L E T V Q R L L P V
L C Q D H G L T P E Q V V A I A S H D G G K
Q A L E T V Q R L L P V L C Q A H G L T P D
Q V V A I A S H D G G K Q A L E T V Q R L L
P V L C Q A H G L T P A Q V V A I A S N G G
G K Q A L E T V Q R L L P V L C Q D H G L T
P D Q V V A I A S N I G G K Q A L E T V Q R
L L P V L C Q D H G L T P E Q V V A I A N N
N G G K Q A L E T V Q R L L P V L C Q A H G
L T P D Q V V A I A S N G G G K Q A L E T V
Q R L L P V L C Q A H G L T P A Q V V A I A
S N G G G K Q A L E T V Q R L L P V L C Q D
H G L T P E Q V V A I A S N I G G R P A L E
S I V A Q L S R P D P A L A A L T N D H L V
A L A C L G G R P A L D A V K K G L P H A P
A L I K R T N R R I P E R T S H R V A G S Q
L V K S E L E E K K S E L R H K L K Y V P H
E Y I E L I E I A R N S T Q D R I L E Met K
V Met E F F Met K V Y G Y R G K H L G G S R
K P D G A I Y T V G S P I D Y G V I V D T K
A Y S G G Y N L P I G Q A D E Met Q R Y V E
E N Q T R N K H I N P N E W W K V Y P S S V
T E F K F L F V S G H F K G N Y K A Q L T R
L N H I T N C N G A V L S V E E L L I G G E
Met I K A G T L T L E E V R R K F N N G E I
N F D I

### TAT-TALE-SacI (Forward 5′):

### DNA:

GCTAGCGCCGCCACCATGGAGCCAGTAGATCCTAGACTAGAGC
CCTGGAAGCATCCAGGAAGTCAGCCTAAAACTGCTTGTACCAA
TTGCTATTGTAAAAAGTGTTGCTTTCATTGCCAAGTTTGTTTC
ATAACAAAAGCCTTAGGCATCTCCtatggcaggaagaagcgga
gacagcgacgaagaGCACATCAGAACAGTCAGACTCATCAAGC
TTCTCTATCAAAGCAACCCACCTCCCAACCCCGAGGGGACCCG
ACAGGCCCGAAGGAAATCGATAAGAAGAAGAGGAAGGTGGGCA
TTCACCGCGGGGTACCTATGGTGGACTTGAGGACACTCGGTTA
TTCGCAACAGCAACAGGAGAAAATCAAGCCTAAGGTCAGGAGC
ACCGTCGCGCAACACCACGAGGCGCTTGTGGGGCATGGCTTCA
CTCATGCGCATATTGTCGCGCTTTCACAGCACCCTGCGGCGCT
TGGGACGGTGGCTGTCAAATACCAAGATATGATTGCGGCCCTG
CCCGAAGCCACGCACGAGGCAATTGTAGGGGTCGGTAAACAGT
GGTCGGGAGCGCGAGCACTTGAGGCGCTGCTGACTGTGGCGGG
TGAGCTTAGGGGGCCTCCGCTCCAGCTCGACACCGGGCAGCTG
CTGAAGATCGCGAAGAGAGGGGGAGTAACAGCGGTAGAGGCAG
TGCACGCCTGGCGCAATGCGCTCACCGGGGCCCCCTTGAACCT
GACCCCAGACCAGGTAGTCGCAATCGCGTCACATGACGGGGGA
AAGCAAGCCCTGGAAACCGTGCAAAGGTTGTTGCCGGTCCTTT
GTCAAGACCACGGCCTTACACCGGAGCAAGTCGTGGCCATTGC
AAGCAATGGGGGTGGCAAACAGGCTCTTGAGACGGTTCAGAGA
CTTCTCCCAGTTCTCTGTCAAGCCCACGGGCTGACTCCCGATC
AAGTTGTAGCGATTGCGTCGCATGACGGAGGGAAACAAGCATT
GGAGACTGTCCAACGGCTCCTTCCCGTGTTGTGTCAAGCCCAC
GGTTTGACGCCTGCACAAGTGGTCGCCATCGCCTCGAATGGCG
GCGGTAAGCAGGCGCTGGAAACAGTACAGCGCCTGCTGCCTGT
ACTGTGCCAGGATCATGGACTGACCCCAGACCAGGTAGTCGCA
ATCGCGAACAATTCGGGGGGAAAGCAAGCCCTGGAAACCGTGC
AAAGGTTGTTGCCGGTCCTTTGTCAAGACCACGGCCTTACACC
GGAGCAAGTCGTGGCCATTGCAAATAATAACGGTGGCAAACAG
GCTCTTGAGACGGTTCAGAGACTTCTCCCAGTTCTCTGTCAAG
CCCACGGGCTGACTCCCGATCAAGTTGTAGCGATTGCGTCCAA
CGGTGGAGGGAAACAAGCATTGGAGACTGTCCAACGGCTCCTT
CCCGTGTTGTGTCAAGCCCACGGTTTGACGCCTGCACAAGTGG
TCGCCATCGCCTCGAATTCGGGCGGTAAGCAGGCGCTGGAAAC
AGTACAGCGCCTGCTGCCTGTACTGTGCCAGGATCATGGACTG
ACCCCAGACCAGGTAGTCGCAATCGCGAACAATAATGGGGGAA
AGCAAGCCCTGGAAACCGTGCAAAGGTTGTTGCCGGTCCTTTG
TCAAGACCACGGCCTTACACCGGAGCAAGTCGTGGCCATTGCA
AATAATAACGGTGGCAAACAGGCTCTTGAGACGGTTCAGAGAC
TTCTCCCAGTTCTCTGTCAAGCCCACGGGCTGACTCCCGATCA
AGTTGTAGCGATTGCGTCGAACATTGGAGGGAAACAAGCATTG
GAGACTGTCCAACGGCTCCTTCCCGTGTTGTGTCAAGCCCACG
GTTTGACGCCTGCACAAGTGGTCGCCATCGCCAGCCATGATGG
CGGTAAGCAGGCGCTGGAAACAGTACAGCGCCTGCTGCCTGTA
CTGTGCCAGGATCATGGACTGACCCCAGACCAGGTAGTCGCAA
TCGCGTCACATGACGGGGGAAAGCAAGCCCTGGAAACCGTGCA
AAGGTTGTTGCCGGTCCTTTGTCAAGACCACGGCCTTACACCG
GAGCAAGTCGTGGCCATTGCAAGCAACATCGGTGGCAAACAGG
CTCTTGAGACGGTTCAGAGACTTCTCCCAGTTCTCTGTCAAGC
CCACGGGCTGACTCCCGATCAAGTTGTAGCGATTGCGAATAAC
AATGGAGGGAAACAAGCATTGGAGACTGTCCAACGGCTCCTTC
CCGTGTTGTGTCAAGCCCACGGTTTGACGCCTGCACAAGTGGT
CGCCATCGCCTCCAATATTGGCGGTAAGCAGGCGCTGGAAACA
GTACAGCGCCTGCTGCCTGTACTGTGCCAGGATCATGGACTGA
CCCCAGACCAGGTAGTCGCAATCGCGTCAAACGGAGGGGGAAA
GCAAGCCCTGGAAACCGTGCAAAGGTTGTTGCCGGTCCTTTGT
CAAGACCACGGCCTTACACCGGAGCAAGTCGTGGCCATTGCAT
CCAATTCGGGTGGCAAACAGGCTCTTGAGACGGTTCAGAGACT
TCTCCCAGTTCTCTGTCAAGCCCACGGGCTGACACCCGAACAG
GTGGTCGCCATTGCTTCTAATGGGGGAGGACGGCCAGCCTTGG
AGTCCATCGTAGCCCAATTGTCCAGGCCCGATCCCGCGTTGGC
TGCGTTAACGAATGACCATCTGGTGGCGTTGGCATGTCTTGGT
GGACGACCCGCGCTCGATGCAGTCAAAAAGGGTCTGCCTCATG
CTCCCGCATTGATCAAAAGAACCAACCGGCGGATTCCCGAGAG
AACTTCCCATCGAGTCGCGGGATCCCTCGAGATGGGCATAACG
ATAAAAAAGAGCACTGCCGAGCAGGTTCTAAGGAAAGCCTACG
AAGCTGCGGCATCAGACGATGTGTTTCTTGAGGACTGGATCTT
TCTGGCTACTAGTCTCCGCGAGGTCGATGCCCCTAGAACATAC
ACCGCCGCGCTAGTCACCGCTTTGCTTGCACGTGCTTGTGACG
ATCGAGTTGATCCCAGGAGCATTAAAGAAAAATATGACGATAG
AGCGTTTTCCTTAAGGACGCTTTGTCATGGGGTAGTTGTACCA
ATGTCGGTTGAGTTAGGATTTGACCTTGGCGCGACAGGAAGAG
AGCCTATAAACAATCAGCCCTTTTTTCGCTACGATCAGTACAG
TGAAATCGTCAGGGTCCAAACCAAAGCCAGACCGTATTTGGAC
CGAGTATCGTCAGCCTTAGCCAGGGTCGATGAAGAGGATTATT
CGACGGAAGAGAGCTTTCGCGCATTAGTAGCGGTGCTCGCGGT
TTGTATCAGTGTTGCGAACAAGAAACAAAGAGTTGCAGTAGGG
TCAGCTATTGTTGAAGCAAGTTTAATCGCAGAGACACAAAGCT
TTGTGGTAAGCGGCCACGACGTTCCTCGGAAGTTGCAAGCCTG
TGTGGCAGCCGGATTGGACATGGTATATAGTGAAGTCGTATCG
CGACGTATAAATGACCCGTCCCGTGATTTTCCTGGGGATGTTC
AGGTAATCTTAGATGGAGACCCATTGCTGACAGTCGAGGTACG
TGGTAAGTCTGTGAGCTGGGAGGGTCTCGAACAATTTGTGTCT
TCAGCAACGTACGCGGGTTTTAGGCGCGTGGCACTAATGGTGG
ATGCGGCTTCCCACGTGTCACTGATGTCTGCTGATGACCTAAC
TTCAGCTTTGGAGCGGAAATATGAGTGTATTGTCAAGGTAAAT
GAGAGCGTCAGTTCCTTTCTCCGAGACGTATTTGTCTGGTCTC
CAAGGGATGTGCATAGTATTCTATCAGCTTTTCCCGAAGCAAT
GTATAGACGGATGATTGAAATAGAAGTACGGGAACCGGAACTG
GACAGATGGGCTGAGATATTTCCAGAAACTGATATC

### Protein:

### Yellow=TAT

### Green=TALE

### Pink=Endonuclease (SacI)

### 5′3′ Frame 1

A S A A T Met E P V D P R L E P W K H P G S
Q P K T A C T N C Y C K K C C F H C Q V C F
I T K A L G I S Y G R K K R R Q R R R A H Q
N S Q T H Q A S L S K Q P T S Q P R G D P T
G P K E I D K K K R K V G I H R G V P [symbol] V
D L R T L G Y S Q Q Q Q E K I K P K V R S T
V A Q H H E A L V G H G F T H A H I V A L S
Q H P A A L G T V A V K Y Q D [symbol] I A A L P
E A T H E A I V G V G K Q W S G A R A L E A
L L T V A G E L R G P P L Q L D T G Q L L K
I A K R G G V T A V E A V H A W R N A L T G
A P L N L T P D Q V V A I A S H D G G K Q A
L E T V Q R L L P V L C Q D H G L T P E Q V
V A I A S N G G G K Q A L E T V Q R L L P V
L C Q A H G L T P D Q V V A I A S H D G G K
Q A L E T V Q R L L P V L C Q A H G L T P A
Q V V A I A S N G G G K Q A L E T V Q R L L
P V L C Q D H G L T P D Q V V A I A N N S G
G K Q A L E T V Q R L L P V L C Q D H G L T
P E Q V V A I A N N N G G K Q A L E T V Q R
L L P V L C Q A H G L T P D Q V V A I A S N
G G G K Q A L E T V Q R L L P V L C Q A H G
L T P A Q V V A I A S N S G G K Q A L E T V
Q R L L P V L C Q D H G L T P D Q V V A I A
N N N G G K Q A L E T V Q R L L P V L C Q D
H G L T P E Q V V A I A N N N G G K Q A L E
T V Q R L L P V L C Q A H G L T P D Q V V A
I A S N I G G K Q A L E T V Q R L L P V L C
Q A H G L T P A Q V V A I A S H D G G K Q A
L E T V Q R L L P V L C Q D H G L T P D Q V
V A I A S H D G G K Q A L E T V Q R L L P V
L C Q D H G L T P E Q V V A I A S N I G G K
Q A L E T V Q R L L P V L C Q A H G L T P D
Q V V A I A N N N G G K Q A L E T V Q R L L
P V L C Q A H G L T P A Q V V A I A S N I G
G K Q A L E T V Q R L L P V L C Q D H G L T
P D Q V V A I A S N G G G K Q A L E T V Q R
L L P V L C Q D H G L T P E Q V V A I A S N
S G G K Q A L E T V Q R L L P V L C Q A H G
L T P E Q V V A I A S N G G G R P A L E S I
V A Q L S R P D P A L A A L T N D H L V A L
A C L G G R P A L D A V K K G L P H A P A L
I K R T N R R I P E R T S H R V A G S L E
Met G I T I K K S T A E Q V L R K A Y E A A
A S D D V F L E D W I F L A T S L R E V D A
P R T Y T A A L V T A L L A R A C D D R V D
P R S I K E K Y D D R A F S L R T L C H G V
V V P Met S V E L G F D L G A T G R E P I N
N Q P F F R Y D Q Y S E I V R V Q T K A R P
Y L D R V S S A L A R V D E E D Y S T E E S
F R A L V A V L A V C I S V A N K K Q R V A
V G S A I V E A S L I A E T Q S F V V S G H
D V P R K L Q A C V A A G L D Met V Y S E V
V S R R I N D P S R D F P G D V Q V I L D G
D P L L T V E V R G K S V S W E G L E Q F V
S S A T Y A G F R R V A L Met V D A A S H V
S L Met S A D D L T S A L E R K Y E C I V K
V N E S V S S F L R D V F V W S P R D V H S
I L S A F P E A Met Y R R Met I E I E V R E
P E L D R W A E I F P E T D I

### TAT-TALE-SacI (Reverse 3′):

### DNA:

GCTAGCGCCGCCACCATGGAGCCAGTAGATCCTAGACTAGAGC
CCTGGAAGCATCCAGGAAGTCAGCCTAAAACTGCTTGTACCAA
TTGCTATTGTAAAAAGTGTTGCTTTCATTGCCAAGTTTGTTTC
ATAACAAAAGCCTTAGGCATCTCCtatggcaggaagaagcgga
gacagcgacgaagaGCACATCAGAACAGTCAGACTCATCAAGC
TTCTCTATCAAAGCAACCCACCTCCCAACCCCGAGGGGACCCG
ACAGGCCCGAAGGAAATCGATAAGAAGAAGAGGAAGGTGGGCA
TTCACCGCGGGGTACCTATGGTGGACTTGAGGACACTCGGTTA
TTCGCAACAGCAACAGGAGAAAATCAAGCCTAAGGTCAGGAGC
ACCGTCGCGCAACACCACGAGGCGCTTGTGGGGCATGGCTTCA
CTCATGCGCATATTGTCGCGCTTTCACAGCACCCTGCGGCGCT
TGGGACGGTGGCTGTCAAATACCAAGATATGATTGCGGCCCTG
CCCGAAGCCACGCACGAGGCAATTGTAGGGGTCGGTAAACAGT
GGTCGGGAGCGCGAGCACTTGAGGCGCTGCTGACTGTGGCGGG
TGAGCTTAGGGGGCCTCCGCTCCAGCTCGACACCGGGCAGCTG
CTGAAGATCGCGAAGAGAGGGGGAGTAACAGCGGTAGAGGCAG
TGCACGCCTGGCGCAATGCGCTCACCGGGGCCCCCTTGAACCT
GACCCCAGACCAGGTAGTCGCAATCGCGTCGAACATTGGGGGA
AAGCAAGCCCTGGAAACCGTGCAAAGGTTGTTGCCGGTCCTTT
GTCAAGACCACGGCCTTACACCGGAGCAAGTCGTGGCCATTGC
AAGCAACATCGGTGGCAAACAGGCTCTTGAGACGGTTCAGAGA
CTTCTCCCAGTTCTCTGTCAAGCCCACGGGCTGACTCCCGATC
AAGTTGTAGCGATTGCGAATAACAATGGAGGGAAACAAGCATT
GGAGACTGTCCAACGGCTCCTTCCCGTGTTGTGTCAAGCCCAC
GGTTTGACGCCTGCACAAGTGGTCGCCATCGCCAGCCATGATG
GCGGTAAGCAGGCGCTGGAAACAGTACAGCGCCTGCTGCCTGT
ACTGTGCCAGGATCATGGACTGACCCCAGACCAGGTAGTCGCA
ATCGCGTCGAACATTGGGGGAAAGCAAGCCCTGGAAACCGTGC
AAAGGTTGTTGCCGGTCCTTTGTCAAGACCACGGCCTTACACC
GGAGCAAGTCGTGGCCATTGCAAATAATAACGGTGGCAAACAG
GCTCTTGAGACGGTTCAGAGACTTCTCCCAGTTCTCTGTCAAG
CCCACGGGCTGACTCCCGATCAAGTTGTAGCGATTGCGTCCAA
CGGTGGAGGGAAACAAGCATTGGAGACTGTCCAACGGCTCCTT
CCCGTGTTGTGTCAAGCCCACGGTTTGACGCCTGCACAAGTGG
TCGCCATCGCCAACAACAACGGCGGTAAGCAGGCGCTGGAAAC
AGTACAGCGCCTGCTGCCTGTACTGTGCCAGGATCATGGACTG
ACCCCAGACCAGGTAGTCGCAATCGCGAACAATAATGGGGGAA
AGCAAGCCCTGGAAACCGTGCAAAGGTTGTTGCCGGTCCTTTG
TCAAGACCACGGCCTTACACCGGAGCAAGTCGTGGCCATTGCA
AATAATAACGGTGGCAAACAGGCTCTTGAGACGGTTCAGAGAC
TTCTCCCAGTTCTCTGTCAAGCCCACGGGCTGACTCCCGATCA
AGTTGTAGCGATTGCGTCCAACGGTGGAGGGAAACAAGCATTG
GAGACTGTCCAACGGCTCCTTCCCGTGTTGTGTCAAGCCCACG
GTTTGACGCCTGCACAAGTGGTCGCCATCGCCTCGAATGGCGG
CGGTAAGCAGGCGCTGGAAACAGTACAGCGCCTGCTGCCTGTA
CTGTGCCAGGATCATGGACTGACCCCAGACCAGGTAGTCGCAA
TCGCGTCACATGACGGGGGAAAGCAAGCCCTGGAAACCGTGCA
AAGGTTGTTGCCGGTCCTTTGTCAAGACCACGGCCTTACACCG
GAGCAAGTCGTGGCCATTGCATCCCACGACGGTGGCAAACAGG
CTCTTGAGACGGTTCAGAGACTTCTCCCAGTTCTCTGTCAAGC
CCACGGGCTGACTCCCGATCAAGTTGTAGCGATTGCGTCGCAT
GACGGAGGGAAACAAGCATTGGAGACTGTCCAACGGCTCCTTC
CCGTGTTGTGTCAAGCCCACGGTTTGACGCCTGCACAAGTGGT
CGCCATCGCCTCGAATGGCGGCGGTAAGCAGGCGCTGGAAACA
GTACAGCGCCTGCTGCCTGTACTGTGCCAGGATCATGGACTGA
CCCCAGACCAGGTAGTCGCAATCGCGTCGAACATTGGGGGAAA
GCAAGCCCTGGAAACCGTGCAAAGGTTGTTGCCGGTCCTTTGT
CAAGACCACGGCCTTACACCGGAGCAAGTCGTGGCCATTGCAA
ATAATAACGGTGGCAAACAGGCTCTTGAGACGGTTCAGAGACT
TCTCCCAGTTCTCTGTCAAGCCCACGGGCTGACTCCCGATCAA
GTTGTAGCGATTGCGTCCAACGGTGGAGGGAAACAAGCATTGG
AGACTGTCCAACGGCTCCTTCCCGTGTTGTGTCAAGCCCACGG
TTTGACGCCTGCACAAGTGGTCGCCATCGCCTCGAATGGCGGC
GGTAAGCAGGCGCTGGAAACAGTACAGCGCCTGCTGCCTGTAC
TGTGCCAGGATCATGGACTGACACCCGAACAGGTGGTCGCCAT
TGCTTCTAACATCGGAGGACGGCCAGCCTTGGAGTCCATCGTA
GCCCAATTGTCCAGGCCCGATCCCGCGTTGGCTGCGTTAACGA
ATGACCATCTGGTGGCGTTGGCATGTCTTGGTGGACGACCCGC
GCTCGATGCAGTCAAAAAGGGTCTGCCTCATGCTCCCGCATTG
ATCAAAAGAACCAACCGGCGGATTCCCGAGAGAACTTCCCATC
GAGTCGCGGGATCCCTCGAGATGGGCATAACGATAAAAAAGAG
CACTGCCGAGCAGGTTCTAAGGAAAGCCTACGAAGCTGCGGCA
TCAGACGATGTGTTTCTTGAGGACTGGATCTTTCTGGCTACTA
GTCTCCGCGAGGTCGATGCCCCTAGAACATACACCGCCGCGCT
AGTCACCGCTTTGCTTGCACGTGCTTGTGACGATCGAGTTGAT
CCCAGGAGCATTAAAGAAAAATATGACGATAGAGCGTTTTCCT
TAAGGACGCTTTGTCATGGGGTAGTTGTACCAATGTCGGTTGA
GTTAGGATTTGACCTTGGCGCGACAGGAAGAGAGCCTATAAAC
AATCAGCCCTTTTTTCGCTACGATCAGTACAGTGAAATCGTCA
GGGTCCAAACCAAAGCCAGACCGTATTTGGACCGAGTATCGTC
AGCCTTAGCCAGGGTCGATGAAGAGGATTATTCGACGGAAGAG
AGCTTTCGCGCATTAGTAGCGGTGCTCGCGGTTTGTATCAGTG
TTGCGAACAAGAAACAAAGAGTTGCAGTAGGGTCAGCTATTGT
TGAAGCAAGTTTAATCGCAGAGACACAAAGCTTTGTGGTAAGC
GGCCACGACGTTCCTCGGAAGTTGCAAGCCTGTGTGGCAGCCG
GATTGGACATGGTATATAGTGAAGTCGTATCGCGACGTATAAA
TGACCCGTCCCGTGATTTTCCTGGGGATGTTCAGGTAATCTTA
GATGGAGACCCATTGCTGACAGTCGAGGTACGTGGTAAGTCTG
TGAGCTGGGAGGGTCTCGAACAATTTGTGTCTTCAGCAACGTA
CGCGGGTTTTAGGCGCGTGGCACTAATGGTGGATGCGGCTTCC
CACGTGTCACTGATGTCTGCTGATGACCTAACTTCAGCTTTGG
AGCGGAAATATGAGTGTATTGTCAAGGTAAATGAGAGCGTCAG
TTCCTTTCTCCGAGACGTATTTGTCTGGTCTCCAAGGGATGTG
CATAGTATTCTATCAGCTTTTCCCGAAGCAATGTATAGACGGA
TGATTGAAATAGAAGTACGGGAACCGGAACTGGACAGATGGGC
TGAGATATTTCCAGAAACTGATATC

### Protein:

### Yellow=TAT

### Green=TALE

### Pink=Endonuclease (SacI)

Met=start methionine amino acid of the protein

### 5′3′ Frame 1

A S A A T Met E P V D P R L E P W K H P G S
Q P K T A C T N C Y C K K C C F H C Q V C F
I T K A L G I S Y G R K K R R Q R R R A H Q
N S Q T H Q A S L S K Q P T S Q P R G D P T
G P K E I D K K K R K V G I H R G V P [symbol] V
D L R T L G Y S Q Q Q Q E K I K P K V R S T
V A Q H H E A L V G H G F T H A H I V A L S
Q H P A A L G T V A V K Y Q D [symbol] I A A L P
E A T H E A I V G V G K Q W S G A R A L E A
L L T V A G E L R G P P L Q L D T G Q L L K
I A K R G G V T A V E A V H A W R N A L T G
A P L N L T P D Q V V A I A S N I G G K Q A
L E T V Q R L L P V L C Q D H G L T P E Q V
V A I A S N I G G K Q A L E T V Q R L L P V
L C Q A H G L T P D Q V V A I A N N N G G K
Q A L E T V Q R L L P V L C Q A H G L T P A
Q V V A I A S H D G G K Q A L E T V Q R L L
P V L C Q D H G L T P D Q V V A I A S N I G
G K Q A L E T V Q R L L P V L C Q D H G L T
P E Q V V A I A N N N G G K Q A L E T V Q R
L L P V L C Q A H G L T P D Q V V A I A S N
G G G K Q A L E T V Q R L L P V L C Q A H G
L T P A Q V V A I A N N N G G K Q A L E T V
Q R L L P V L C Q D H G L T P D Q V V A I A
N N N G G K Q A L E T V Q R L L P V L C Q D
H G L T P E Q V V A I A N N N G G K Q A L E
T V Q R L L P V L C Q A H G L T P D Q V V A
I A S N G G G K Q A L E T V Q R L L P V L C
Q A H G L T P A Q V V A I A S N G G G K Q A
L E T V Q R L L P V L C Q D H G L T P D Q V
V A I A S H D G G K Q A L E T V Q R L L P V
L C Q D H G L T P E Q V V A I A S H D G G K
Q A L E T V Q R L L P V L C Q A H G L T P D
Q V V A I A S H D G G K Q A L E T V Q R L L
P V L C Q A H G L T P A Q V V A I A S N G G
G K Q A L E T V Q R L L P V L C Q D H G L T
P D Q V V A I A S N I G G K Q A L E T V Q R
L L P V L C Q D H G L T P E Q V V A I A N N
N G G K Q A L E T V Q R L L P V L C Q A H G
L T P D Q V V A I A S N G G G K Q A L E T V
Q R L L P V L C Q A H G L T P A Q V V A I A
S N G G G K Q A L E T V Q R L L P V L C Q D
H G L T P E Q V V A I A S N I G G R P A L E
S I V A Q L S R P D P A L A A L T N D H L V
A L A C L G G R P A L D A V K K G L P H A P
A L I K R T N R R I P E R T S H R V A G S L
E Met G I T I K K S T A E Q V L R K A Y E A
A A S D D V F L E D W I F L A T S L R E V D
A P R T Y T A A L V T A L L A R A C D D R V
D P R S I K E K Y D D R A F S L R T L C H G
V V V P Met S V E L G F D L G A T G R E P I
N N Q P F F R Y D Q Y S E I V R V Q T K A R
P Y L D R V S S A L A R V D E E D Y S T E E
S F R A L V A V L A V C I S V A N K K Q R V
A V G S A I V E A S L I A E T Q S F V V S G
H D V P R K L Q A C V A A G L D Met V Y S E
V V S R R I N D P S R D F P G D V Q V I L D
G D P L L T V E V R G K S V S W E G L E Q F
V S S A T Y A G F R R V A L Met V D A A S H
V S L Met S A D D L T S A L E R K Y E C I V
K V N E S V S S F L R D V F V W S P R D V H
S I L S A F P E A Met Y R R Met I E I E V R
E P E L D R W A E I F P E T D I

### Summary of Other Uses of P2E2 Constructs.

Above is a description of how to target a specific region of HIV-1 B subtype viruses. The proviral DNA sequence of HIV-1 & 2 viruses can be found in the Los Alamos HIV compendium (http://www.hiv.lanl.gov/content/sequence/HIV/COMPENDIUM/compendium.html). Targeting signals that are highly conserved in the 5′UTR of HIV-1 & 2 could provide additional ways to prevent HIV replication. While this version is focused on HIV-2 subtype B, versions that target all HIV-1, HIV-2 and SIV viruses, or subtype specific viruses, could be made by the same approach. In addition, the P2E2 constructs have applications that reach well beyond the example of HIV given here. The most obvious expansion in applying this technology could be used to target the removal of pieces of other DNA-based genomes of viruses from host cells. For example, hepatitis or bird flu.

Other types of infectious disease that could be targeted are bacteria. A sequenced genome of any pathogen can be used to identify genes that are essential for their viability, and the unique genomic regions flanking or disruption the essential gene. These could be targeted by P2E2 constructs to delete the region of the pathogen (virus, bacterium, or single celled eukaryotic parasite) genome. Likewise, some bacteria use plasmids that can encode virulent genes that could be targeted in the same way. Another approach could be to use the P2E2 constructs to delete the origin of replication to prevent duplication of the plasmid, or similarly for the bacterial chromosome. Finally, we would also target the multidrug resistance transporter used to pump antibiotics outside of the bacterium.

Other applications include a means of fighting non-infectious diseases. In many diseases, patients have genes with bad alleles, which causes proteins to misfold resulting in pathologies. Examples are Lewy bodies in Parkinson's disease, amyloid plaques in Alzheimer's disease, and protein insolubility in triplet repeat diseases such as in Huntington's disease. Since many of these disease-causing alleles are in genes that are not essential, they can potentially be deleted or disrupted to prevent expression of the precipitating protein.

This technology also may be used to target cancer. One approach would be to target proto-oncogenes and oncogenes by local introduction of the P2E2 constructs into the local region of the tumor. Another approach would by disabling endogenous apoptosis inhibitors such as BAD and Bcl2 in host cells with the goal of encouraging apoptosis of cancer cells. This could also be used to treat other disease where induction of apoptosis of specific cells is desirable. In these cases the P2E2 constructs could be injected into specific locations to induced apoptosis of all local cells. Alternatively, and likely more desirably, we could use cell-specific and/or inducible promoters to target specific cell types for removal of a specific DNA region. The example pCDNA plasmid vector for the HIV targeting construct has a CMV promoter element to target all cell types, which could be replaced with cell-specific or inducible promoter. Other approaches could be to target deletion of the centromere of specific chromosomes to reduce zygosity. This would be a reasonable strategy in treating trisomy 21 (as observed in Down Syndrome). We could also possible treat autoimmune diseases. The P2E2 construct could be used to removing specific harmful antibodies that generate immune responses in the 100's of autoimmune diseases such as type 2 Diabetes and Lupus. This could also be useful in treating sever obesity by targeting the Ghrelin gene to reduce hunger. There is also the potential to target several genes for reducing hyperthyroidisms without surgery. Yet another future method would be to employ a “cocktail” of P2E2 construct pairs to cut multiple targets.

To test the ability of proposed protein pairs (that is, pairs of both cell penetrating peptide and DNA binding domain-nuclease) to bind and cleave target DNA, one must first build DNA constructs. These DNA constructs will be used by cellular machinery as a blue print for making RNAs. The newly synthesized RNAs will then be used as a blue print for making the actual proteins. To build the DNA constructs, we must insert the DNA sequence coding for the protein components into a “vehicle” that the cellular machinery can use in the synthesis process. This vehicle is a DNA vector. We have built control DNA constructs for the 5′Tal-FokI and the 3′Tal-FokI to exemplify the generic concept and provide an illustrative working example that will enable performance and use of this technology with any synthesize protein pair for targeting any target DNA to be cleaved.

To make the desired protein that contains the DNA binding portion fused to the DNA cutting portion, the DNA construct must be transcribed into RNA. That RNA is then translated into protein according to the following procedure.

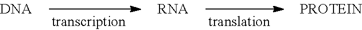

RNA polymerase, a type of enzyme, is a component of the necessary cellular machinery that uses DNA as a blue print (template) in RNA synthesis (transcription). Once the RNA has been synthesized from the DNA template, the RNA can be used as a template by the ribosome (another type of enzyme) in the process of protein synthesis (translation).

Commercial kits are available that allow researchers to transcribe their DNA constructs into RNAs, which can then be translated into proteins, all within a single test tube reaction. We added our DNA constructs to TNT Quick Coupled Transcription/Translation system reactions (Promega) to make our desired proteins (5′Tal-FokI and 3′Tal-FokI). To visualize that our proteins had been made, samples of the test tube reactions were run on a protein gel that separates proteins according to size. The protein gel was then “transferred” onto a blot (membrane). This blot now contains all of the proteins from the protein gel. To confirm the identities of our desired proteins, we “probed” the blot with specific primary antibodies that recognize and bind to our proteins. Treatment with secondary antibodies follows, where in the secondary antibodies recognize and bind the primary antibodies. Because the secondary antibodies have a specific enzymatic activity, we can add a chemical substrate to the blot and the secondary antibodies will create a “glow” on the blot areas where our protein is found. If our proteins are present, they will appear under the camera filter as dark bands. As seen below our proteins appear in sample lanes 2 (5′Tal-FokI), 3 (3′Tal-FokI), and 4 (5′Tal-FokI and 3′Tal-FokI) as concentrated dark bands. Because no DNA constructs were added to sample 1, none our desired protein should have been made, therefore there should not be any concentrated signal in lane 1 (which is the case).

This example confirmed that our DNA constructs were functional blue prints that can be used by cellular machinery to produce RNA. That RNA was a functional template that could then be used by cellular machinery to synthesize the desired proteins (5′Tal-FokI and 3′Tal-FokI).

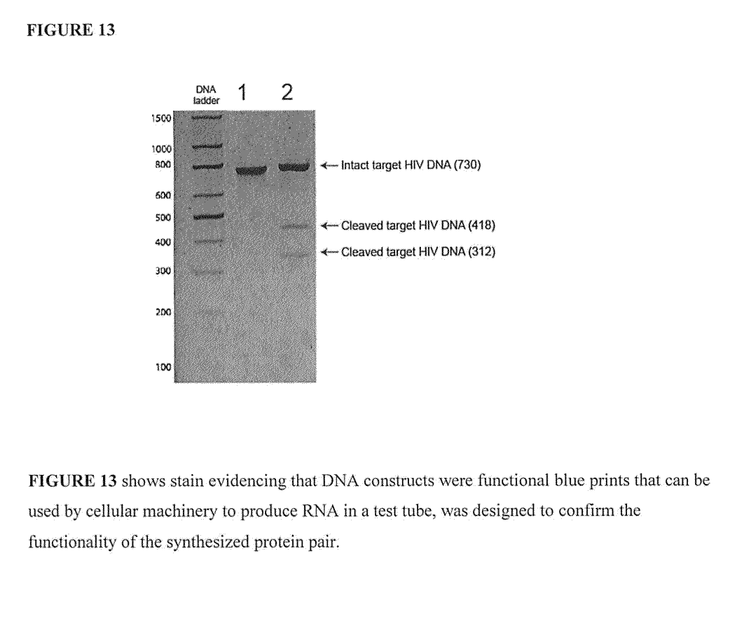

The next example, which was performed in a test tube, was designed to confirm the functionality of the synthesized protein pair (i.e., ability of the proteins to bind and cleave the HIV-1 DNA target sequence). The results are shown in FIG. 13.

That example determined functionality of the 5′Tal-FokI and 3′Tal-FokI proteins (i.e., ability to bind and cleave target HIV-1 DNA). The 5′Tal-FokI and 3′Tal-FokI proteins were synthesized using the test tube transcription/translation reactions. These reactions were supplemented with target HIV-1 DNA and added to cleavage assay buffer to promote cleavage of the target HIV-1 DNA. To determine whether the 3′Tal-FokI and 5′Tal-FokI paired proteins were able to cleave the target HIV-1 DNA, the input target DNA was purified (isolated) from the cleavage reaction using a DNA purification kit (5′PRIME kit). Following purification, the target HIV-1 DNA was loaded into a DNA-agarose gel to visualize the DNA based on size. If the target HIV-1 DNA was intact (i.e. not cleaved by the Tal-FokI proteins), it would appear as one band on the DNA-agarose gel at position 730. If all of the target HIV-1 DNA was cleaved by the paired Tal-FokI proteins, two bands would appear on the DNA-agarose gel at positions 418 and 312. If only a portion of the target HIV-1 DNA was cleaved by the paired Tal-FokI proteins, three bands would appear on the gel: Band 1 corresponding to the intact band at position 730 and Bands 2 and 3 corresponding to the cleaved product at positions 418 and 312. The DNA ladder lane in the DNA agarose gel below contains a DNA ladder to be used to visualize DNA band size. Lane 1 contains target HIV-1 DNA purified from a cleavage reaction that did not contain the paired Tal-FokI proteins. Lane 2 contains target HIV-1 DNA purified from a cleavage reaction that contained the paired Tal-FokI proteins. As illustrated, the presence of the paired Tal-FokI proteins resulted in three bands: the first at position 730 corresponding to the intact target HIV-1 DNA, and the second (418) and third (312) corresponding to the cleaved target HIV-1 DNA.

This experiment confirmed that the Tal-FokI proteins synthesized in the test tube reactions were able to cleave the target HIV-1 DNA in a predicted manner (i.e., DNA agarose band pattern).

The next experiment performed with the control Tal-FokI pair involved placing the 5′Tal-FokI and 3′Tal-FokI DNA constructs into mammalian cells that contained two integrated copies of HIV-1 proviral target DNA. The goal of this “in vivo” experiment was to determine if the basic Tal-FokI proteins could cleave the HIV-1 proviral target DNA without the need to “wake” the cell up (i.e. make the cells leave the latent state and start actively producing viral components).

### Example 1

This example was performed to determine if the basic Tal-FokI protein pair (i.e., lacking the cell penetrating peptide (Tat)) could bind and cleave integrated target HIV proviral DNA in a cell (in vivo). It has been shown that basic Tal-FokI protein pairs can have difficulty inducing mutagenicity of cellular DNA by binding/cleaving due to the presence of methyl groups (methylation) on the cellular DNA target (Chen et al 2013, NAR). Because integrated HIV-1 proviral DNA in latent cell lines such as U1/HIV-1 is methylated (Ishida et al 2006, Retrovirology), we would predict that the basic Tal-FokI protein pair would be unable to introduce mutagenicity at a significant level. However, we would predict that a Tat-Tal-FokI protein pair would be able to introduce mutagenicity because the presence of the Tat protein has been shown to affect the methylation state of HIV-1 proviral DNA in U1/HIV-1 cells (Emiliani et al 1998, J Virology). To that end, the 5′Tal-FokI and 3′Tal-FokI DNA constructs were placed (transfected) into U1/HIV-1 cells. U1/HIV-1 cells are promonocyte cells that contain two copies of HIV-1 proviral DNA. Once the Tal-FokI DNA constructs are in the mammalian cells, RNA synthesis and protein production of the Tal-FokI protein pair are under the control of cellular machinery. If the Tal-FokI protein pair is able to bind and cleave the integrated target HIV-1 DNA, the cellular machinery will attempt to “fix” the cleavage break in the target HIV-1 proviral DNA but in a way that is easily detectable using DNA sequencing (i.e. it makes mistakes such as insertions or deletions of DNA sequence). To that end, we placed both 5′Tal-FokI and 3′Tal-FokI DNA constructs into U1/HIV-1 cells and then allowed 48 hours for protein expression. At the end of 48 hours, the U1/HIV-1 cells were collected, broken open (lysed) and the genomic DNA therein extracted. This genomic DNA was isolated (purified) using a commercial genomic DNA purification kit (Invitrogen). Once the genomic DNA was purified, polymerase chain reactions (pcrs) were performed to amplify (make many copies) the targeted region of the HIV-1 proviral DNA. The copies of the targeted region were then individually inserted (ligated) into a vector and transformed into bacteria. Once in the bacteria, many copies of this DNA were made and then extracted using a DNA isolation kit (Qiagen). These DNAs were then sent for DNA sequencing (Beckman Coulter) so that any indication of cleavage by the Tal-FokI proteins (insertions or deletions of DNA in the target site) could be detected. As seen on the next page, in the DNA sequence alignment the 5′Tal-FokI DNA binding site is highlighted in yellow while the 3′Tal-FokI DNA binding site is highlighted in green. The target cleavage area is bolded in black. The asterisk found below the HIV1NY5 indicates that all of the DNA sequences (3A1-3A10) are identical (have the same nucleotide) at that position with regard to the reference U1/HIV-1 DNA sequence (HIVINY5). The only exception of a single DNA base change (A to G) is in sample 3A6, the red “G” found outside of the target region. This is not indicative of successful cleavage by the Tal-FokI proteins, followed by DNA repair by the cellular machinery. This result supports our hypothesis that the control Tal-FokI protein pair would not be able to bind/cleave the target HIV-1 DNA region at a detectable level.

| | | |
|---|---|---|
| CS730-3A4_pGEX5 | 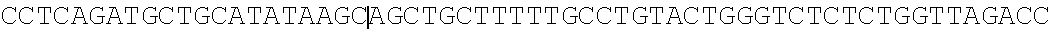 | 434 |
| CS730-3A8_pGEX5 | CCTCAGATGCTGCATATAAGCAGCTGCTTTTTGCCTGTACTGGGTCTCTCTGGTTAGACC | 430 |
| CS730-3A2_pGEX5 | CCTCAGATGCTGCATATAAGCAGCTGCTTTTTGCCTGTACTGGGTCTCTCTGGTTAGACC | 427 |
| CS730-3A10_pGEX5 | CCTCAGATGCTGCATATAAGCAGCTGCTTTTTGCCTGTACTGGGTCTCTCTGGTTAGACC | 423 |
| CS730-3A3_pGEX5 | CCTCAGATGCTGCATATAAGCAGCTGCTTTTTGCCTGTACTGGGTCTCTCTGGTTAGACC | 422 |
| CS730-3A6_pGEX5 | CCTCAGATGCTGCATATAGGCAGCTGCTTTTTGCCTGTACTGGGTCTCTCTGGTTAGACC | 416 |
| CS730-3A5_pGEX5 | CCTCAGATGCTGCATATAAGCAGCTGCTTTTTGCCTGTACTGGGTCTCTCTGGTTAGACC | 415 |
| CS730-3A7_pGEX5 | CCTCAGATGCTGCATATAAGCAGCTGCTTTTTGCCTGTACTGGGTCTCTCTGGTTAGACC | 415 |
| CS730-3A1_pGEX5 | CCTCAGATGCTGCATATAAGCAGCTGCTTTTTGCCTGTACTGGGTCTCTCTGGTTAGACC | 412 |
| CS730-3A9_pGEX5 | CCTCAGATGCTGCATATAAGCAGCTGCTTTTTGCCTGTACTGGGTCTCTCTGGTTAGACC | 408 |
| HIV1NY5 | CCTCAGATGCTGCATATAAGCAGCTGCTTTTTGCCTGTACTGGGTCTCTCTGGTTAGACC | 472 |
| | ****************** ***************************************** | |
| CS730-3A4_pGEX5 | 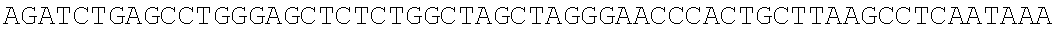 | 494 |
| CS730-3A8_pGEX5 | AGATCTGAGCCTGGGAGCTCTCTGGCTAGCTAGGGAACCCACTGCTTAAGCCTCAATAAA | 490 |
| CS730-3A2_pGEX5 | AGATCTGAGCCTGGGAGCTCTCTGGCTAGCTAGGGAACCCACTGCTTAAGCCTCAATAAA | 487 |
| CS730-3A10_pGEX5 | AGATCTGAGCCTGGGAGCTCTCTGGCTAGCTAGGGAACCCACTGCTTAAGCCTCAATAAA | 483 |
| CS730-3A3_pGEX5 | AGATCTGAGCCTGGGAGCTCTCTGGCTAGCTAGGGAACCCACTGCTTAAGCCTCAATAAA | 482 |
| CS730-3A6_pGEX5 | AGATCTGAGCCTGGGAGCTCTCTGGCTAGCTAGGGAACCCACTGCTTAAGCCTCAATAAA | 476 |
| CS730-3A5_pGEX5 | AGATCTGAGCCTGGGAGCTCTCTGGCTAGCTAGGGAACCCACTGCTTAAGCCTCAATAAA | 475 |
| CS730-2A7_pGEX5 | AGATCTGAGCCTGGGAGCTCTCTGGCTAGCTAGGGAACCCACTGCTTAAGCCTCAATAAA | 475 |
| CS730-3A1_pGEX5 | AGATCTGAGCCTGGGAGCTCTCTGGCTAGCTAGGGAACCCACTGCTTAAGCCTCAATAAA | 472 |
| CS730-3A9_pGEX5 | AGATCTGAGCCTGGGAGCTCTCTGGCTAGCTAGGGAACCCACTGCTTAAGCCTCAATAAA | 468 |
| HIV1NY5 | AGATCTGAGCCTGGGAGCTCTCTGGCTAGCTAGGGAACCCACTGCTTAAGCCTCAATAAA | 532 |
| | ************************************************************ | |

There are other applications not related to disease. There is the potential to use this technology to remove diseased alleles from gametes, change a persons blood type to “O” that of a universal donor. This has implications in organ transplantation and rejection. Essential genes in pests such as insects (e.g. Africanized killer bees and mosquitos) and rodents could be targeted. Likewise, key gene for reproduction could be targeted to create infertile animals or as a means of birth control. This could be exploited even further by creating recombinant organisms having inserted tags that flank essential genes. Thus, one could use the technology introduce a recombinant bacteria designed to clean up an oil spill, and then selectively kill of the organism when the job is complete.

### Example 2

An effort was made here to identify a strong HIV proviral DNA target for binding and cleavage by a protein pair. DNA sequence alignments were performed on 226 DNA sequences of the 5′ Long Terminal Repeat (LTR) region of HIV-1 type B sequences. The 5′ LTR was selected because of its high level of nucleotide conservation among HIV-1 viruses. The identified binding and cleavage region selected based on conservation is depicted below. The bold font denotes binding regions while the underlined font denotes cleavage regions and the lower case lettering identifies the specific targets.

5′ Tale Target /endonuclease target/
TCTCTGGTTAGACCATCT/GAGCCTGGgagctcTCTGGC/TAACTAGGG
AACCCACTGCTTA
endonuclease target 3′ Tale Target
3′ AGAGACCAATCTGGTCTAGA/CTCGGACCctcgagAGACCG/ATTG
ATCCCTTGGGTGACGAAT

These regions were selected based on high levels of conservation as illustrated in the tables below. The horizontal (x-axis) nucleotide sequence represents the HIV-1 sequence (master sequence) that the other 225 HIV-1 sequences were aligned to using the sequence alignment program. The % vertical nucleotides (y-axis) represent the four-nucleotide possibilities that could be found in a DNA sequence. In addition, percentages of DNA sequences that match the master sequence nucleotide at that position are shown.

5′ TALE Binding Target

| | T | C | T | C | T | G | G | T | T | A | G | A | C | C | A | G | A | T | C | T |
|---|---|---|---|---|---|---|---|---|---|---|---|---|---|---|---|---|---|---|---|---|
| % A | 0 | 0 | 0 | 0 | 0 | 11.5 | 0 | 0.4 | 0 | 98.2 | 0 | 99.6 | 0 | 0 | 100 | 0 | 100 | 0 | 0 | 6.6 |
| % T | 100 | 0 | 100 | 0 | 100 | 21.2 | 0 | 99.6 | 95.6 | 0 | 0 | 0 | 0.9 | 0 | 0 | 0 | 0 | 100 | 1.8 | 92.5 |
| % G | 0 | 0 | 0 | 0 | 0 | 67.3 | 100 | 0 | 0 | 1.8 | 100 | 0 | 0 | 0 | 0 | 100 | 0 | 0 | 0 | 0 |
| % C | 0 | 100 | 0 | 100 | 0 | 0 | 0 | 0 | 4.4 | 0 | 0 | 0.4 | 99.1 | 100 | 0 | 0 | 0 | 0 | 96.2 | 0.9 |
| % Consv. | 100 | 100 | 100 | 100 | 100 | 67.3 | 100 | 99.6 | 99.6 | 98 | 100 | 99.6 | 99.6 | 100 | 100 | 100 | 100 | 100 | 96.2 | 92.5 |

3′ TALE Binding Target

| | A | T | T | G | A | T | C | C | C | T | T | G |
|---|---|---|---|---|---|---|---|---|---|---|---|---|
| % A | 99.6 | 0 | 0 | 0.4 | 100 | 0 | 0 | 0 | 0 | 0 | 0 | 0 |
| % T | 0 | 92 | 69.3 | 0 | 0 | 99.1 | 0 | 0.4 | 0.9 | 99.1 | 100 | 0 |
| % G | 0.4 | 0 | 0 | 99.6 | 0 | 0 | 0 | 0 | 0 | 0 | 0 | 100 |
| % C | 0 | [symbol] | 30.7 | 0 | 0 | 0.9 | 100 | 99.6 | 99.1 | 0.9 | 0 | 0 |
| % | 99.6 | 92 | 69.3 | 99.6 | 100 | 99.1 | 100 | 99.6 | 99.1 | 99.1 | 100 | 100 |
| Consv. | | | | | | | | | | | | |

| | G | G | T | G | A | C | G | A | A | T |
|---|---|---|---|---|---|---|---|---|---|---|
| % A | 0 | 0 | 0 | 0 | 100 | 0 | 0 | 100 | 100 | 0 |
| % T | 0 | 0 | 100 | 0 | 0 | 0 | 0 | 0 | 0 | 100 |
| % G | 100 | 100 | 0 | 100 | 0 | 0 | 100 | 0 | 0 | 0 |
| % C | 0 | 0 | 0 | 0 | 0 | 100 | 0 | 0 | 0 | 0 |
| % | 100 | 100 | 100 | 100 | 100 | 100 | 100 | 100 | 100 | 100 |
| Consv. | | | | | | | | | | |

[symbol]

indicates data missing or illegible when filed

As illustrated above, the proposed binding regions are for the most part highly conserved. To test the ability of the proposed protein pair (cell penetrating peptide-DNA binding domain-nuclease) to bind and cleave the proposed HIV target DNA, one must first build DNA constructs. We generated at least some of the constructs using a Gibson assembly of synthetic Gblocks which were purchased from commercial sources. It is also possible to use the protocols and DNAS provided by the Joung lab Real Assembly™ kit to make the constructs. These protocols are incorporated herein by reference, even though they are publicly available information known to those skilled in the art. These DNA constructs will be used by cellular machinery as a blue print for making RNAs. The newly synthesized RNAs will then be used as a blue print for making the actual proteins. To build the DNA constructs, we must glue the DNA insert sequence coding for the DNA binding domain protein components into a “vehicle” that the cellular machinery can use in the synthesis process. This vehicle is a DNA vector as exemplified in FIG. 1.

The 5′ Tale DNA construct will produce proteins that target “TCTCTGGTrAGACCAGATCT” for binding while the 3′ Tale DNA construct will produce proteins that target “TAAGCAGTGGGTTCCCTAGTTA” for binding. The pairs of constructs containing the FokI catalytic core will produce proteins that target within the “GAGCCTGGGAGCTCTCTGGC” of the underlined or bold region for cutting.

To make the desired proteins that contain the DNA binding portion fused to the DNA cutting portion, the DNA construct must be transcribed into RNA. That RNA is then translated into protein as shown below.

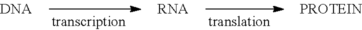

RNA polymerase, a type of enzyme, is a component of the necessary cellular machinery that uses DNA as a blue print (template) in RNA synthesis (transcription). Once the RNA has been synthesized from the DNA template, the RNA can be used as a template by the ribosome (another type of enzyme) in the process of protein synthesis (translation).

Researchers are able to transcribe their DNA constructs into RNAs, which can then be translated into proteins, all within a single test tube (batch) reaction. The test tube reactions contain materials necessary for transcription, including the DNA template to be transcribed, RNA polymerase, nucleotides, salts, and ribonuclease inhibitors in addition to materials necessary for translation including amino acids, tRNA, ribosomes, and initiation/elongation/termination factors (all found in the rabbit reticulocyte lysate added to the tube).

To visualize that the targeted proteins had been made, samples of the test tube reactions were run on a 4-12% Bis-Tris protein gel at 125 volts for 1-1.5 hours to separate proteins according to size. The protein gel was then “transferred” using an electrical current for two hours at 400 milliamps onto a polyvinylidene difluoride (PVDF) membrane. This membrane then contained all of the proteins from the protein gel. The proteins are transferred onto a membrane to allow confirmation of protein identity using antibodies against the desired protein. To confirm the identities of our desired proteins, the membrane must be first “blocked” with a 5% milk solution (1 gram of milk powder plus 20 mL 1×TTBS) for 1 hour at room temperature on a shaker. Blocking the membrane with the milk solution prevents the antibody from binding directly to the membrane; instead the antibody must recognize and bind the desired protein. The membrane is then “washed” on a shaker with 1×TTBS for 15 minutes. This wash is repeated two times. To visualize the targeted proteins, a “protein sandwich” would be constructed, consisting of the target protein, a primary antibody, and secondary antibody. The secondary antibody will catalyze a reaction (oxidation) of a substrate to produce light. This light will be detected by a CCD camera, producing an “image” i.e., band of the target protein, as shown in FIG. 3.

To do this, the membrane is incubated with the specific primary antibody that recognizes and binds to our proteins, based on the presence of a FLAG tag contained within our proteins (i.e., the presence of the following amino acid sequence in the protein: DYKDDDDK). The membrane is sealed in a plastic bag with 1 mL of 1×TTBS and 3.3 μL of rabbit anti-Flag antibody and incubated overnight at 4° C. on a shaking platform. The next morning the membrane is washed with 1×TTBS on a shaker for 15 minutes and the wash is repeated two times. The membrane is then treated with a secondary antibody. The secondary antibody recognizes and binds to the primary antibody; in this case a goat anti-rabbit horseradish peroxidase antibody was applied. The membrane was incubated in a container at room temperature with 1 μL goat anti-rabbit horseradish peroxidase in 20 mL 1×TTBS for 1 hour. The membrane was then washed with 1×TTBS for 15 minutes, with the wash being repeated twice. In order to visualize the protein “sandwich” consisting of the desired protein bound to the primary antibody, which is bound to the secondary antibody, a solution containing luminol and hydrogen peroxide is applied. The horseradish peroxidase portion of the secondary antibody will catalyze the oxidation of luminol by peroxide. The product produced from this reaction emits light at 425 nm, and can be visually captured using a CCD camera. If our proteins are present, they will appear under the camera filter as bands. As seen below our proteins appear in sample lanes 2 (5′Tal-FokI), 3 (3′Tal-FokI), and 4 (5′Tal-FokI and 3′Tal-FokI) as concentrated dark bands. Because no DNA constructs were added to sample 1, none our desired protein should have been made, therefore there should not be any concentrated signal in lane 1 (which is the case), as shown in FIG. 5.

This experiment confirmed that our DNA constructs were functional blue prints that can be used by cellular machinery to produce RNA. That RNA was a functional template that could then be used by cellular machinery to synthesize the desired proteins (5′Tal-FokI and 3′Tal-FokI).

The next experiment performed in a test tube was designed to confirm the functionality of the synthesized protein pair (i.e. ability of the proteins to bind and cleave the HIV-1 DNA target sequence).

### Example 2

The next example determined functionality of the 5′Tal-FokI and 3′Tal-FokI proteins (i.e., ability to bind and cleave target HIV-1 DNA). The 5′Tal-FokI and 3′Tal-FokI proteins were synthesized in a test tube. The synthesis reactions contained 250 ng of each of the 5′TalFokI and 3′TalFokI DNA templates, 0.5 μL methionine (1 mM), and 20 μL of rabbit reticulocyte lysate. The rabbit reticulocyte lysate contained RNA polymerases, nucleotides, salts, ribonuclease inhibitors, amino acids, tRNA, ribosomes, and initiation/elongation/termination factors. In addition, these reactions were supplemented with 500 ng of target HIV-1 DNA. These transcription/translation reactions were incubated at 30° C. for 2 hours. At the end of the incubation period, approximately 23 μL of the 25 μL transcription/translation reaction was added to a tube containing 100 μL of cleavage assay buffer (20 mM Tris-HCl, 5 mM magnesium chloride, 50 mM potassium chloride, 5% glycerol and 0.5 mg/mL bovine serum albumin). This tube was then incubated at 30° C. for 4 hours to promote cleavage of the target HIV-1 DNA by the 5′&3′Tal-FokI protein pairs. At the end of the cleavage reaction, 0.5 μL of RNase was added to the reaction and the reaction was incubated at 30° C. for 15 minutes. This step was performed to degrade the RNA present in the reaction to make visualization of the DNA on an agarose gel easier.

To determine whether the 3′Tal-FokI and 5′Tal-FokI paired proteins were able to cleave the target HIV-1 DNA, the input target DNA was purified (isolated) from the cleavage reaction. To purify the target DNA, 625 μL of a high salt buffer (guanidinium chloride, propan-2-ol) was mixed with the cleavage reaction. This solution was then applied to a silica-gel membrane column. The high salt conditions allowed for the DNA to bind to this membrane. Once the DNA was bound, the column was washed twice with a buffer containing ethanol. After removing residual ethanol from the column by centrifugation of the column, the DNA was eluted off of the column using an elution buffer containing 10 mM Tris-HCl, pH 8.5. The eluted DNA volume of 50 μL is larger than desired for agarose gel electrophoresis analysis; therefore the DNA had been combined with glycogen, 3M sodium acetate, and 95% ethanol to concentrate the DNA. This solution was then precipitated at −20° C. for 2 hours. Following this incubation period, the samples were centrifuged to pellet the DNA. The DNA pellet was washed with 75% ethanol solution to remove excess salt, air dried to remove excess ethanol, and then resuspended in a 10 μL volume of water. Following precipitation, the 10 μL of target HIV-1 DNA was combined with 2 μL of 6× DNA loading buffer (25 mg xylene, 25 mg bromophenol blue, 6.7 mL autoclaved water, 3.3 mL glycerol) and then loaded into a well of a 2% DNA-agarose gel (1.2 g agarose, 60 mL 1×TAE buffer (40 mM Tris acetate, 1 mM EDTA) to visualize the DNA based on size. An electric current was applied to the submerged gel in the gel apparatus (125 volts for 1.5 hrs). Because DNA has an overall negative charge, it will migrate away from the negative anode towards the positively charged anode. The gel provides a honeycomb network for the DNA to migrate through, with smaller pieces of DNA moving faster than larger pieces, allowing for separation of DNA based on size. The DNA was visualized using ethidium bromide, a fluorescent dye that intercalates with DNA. This dye glows pink under a UV light. A CCD camera with a UV light was used to capture an image of the gel.

With regard to the target DNA, if the target HIV-1 DNA was intact (i.e., not cleaved by the paired Tal-FokI proteins), it would appear as one band on the DNA agarose gel at position 730 (with reference to the DNA ladder). If all of the target HIV-1 DNA was cleaved by the paired Tal-FokI proteins, two bands would appear on the DNA agarose gel at positions 418 and 312. If only a portion of the target HIV-1 DNA was cleaved by the paired Tal-FokI proteins, three bands would appear on the gel: Band 1 corresponding to the intact band at position 730 and Bands 2 and 3 corresponding to the cleaved product at positions 418 and 312. The DNA ladder lane in the DNA agarose gel below contains DNAs of different sizes to be used to visualize DNA band size. Lane 1 contains target HIV-1 DNA purified from a cleavage reaction that did not contain the paired Tal-FokI proteins. Lane 2 contains target HIV-1 DNA purified from a cleavage reaction that contained the paired Tal-FokI proteins. As illustrated in FIG. 4, the presence of the paired Tal-FokI proteins resulted in three bands: the first at position 730 corresponding to the intact target HIV-1 DNA, and the second (418) and third (312) corresponding to the cleaved target HIV-1 DNA, as shown in FIG. 4.

This Example 2 confirmed that the Tal-FokI proteins synthesized in the test tube reactions were able to cleave the target HIV-1 DNA in a predicted manner (i.e., DNA agarose band pattern).

### Example 3

The next example was performed with the control Tal-FokI pair and involved placing the 5′Tal-FokI and 3′Tal-FokI DNA constructs into mammalian cells that contained two integrated copies of HIV-1 proviral target DNA. The goal of this “in vivo” example was to determine if the basic Tal-FokI proteins could cleave the HIV-1 proviral target DNA without the need to “wake” the cell up (i.e., make the cells leave the latent state and start actively producing viral components). This example was performed to determine if the basic Tal-FokI protein pair (i.e., lacking the cell penetrating peptide (Tat)) could bind and cleave integrated target HIV proviral DNA in a cell (in vivo). It has been shown that basic Tal-FokI protein pairs can have difficulty inducing mutagenicity of cellular DNA by binding/cleaving due to the presence of methyl groups (methylation) on the cellular DNA target (Chen et al 2013, NAR). Because integrated HIV-1 proviral DNA in latent cell lines such as U1/HIV-1 is methylated (Ishida et al 2006, Retrovirology), we would predict that the basic Tal-FokI protein pair would be unable to introduce mutagenicity at a significant level. However, we would predict that a Tat-Tal-FokI protein pair would be able to introduce mutagenicity because the presence of the Tat protein has been shown to affect the methylation state of HIV-1 proviral DNA in U1/HIV-1 cells (Emiliani et al 1998, J Virology). To that end, the 5′Tal-FokI and 3′Tal-FokI DNA constructs were placed (transfected) into U1/HIV-1 cells. U1/HIV-1 cells are promonocyte cells that contain two copies of HIV-1 proviral DNA. To transfect the 5′Tal-FokI and 3′TalFokI DNA constructs into U1/HIV-1 cells, approximately 250 ng of each DNA construct is added to 100 μL of serum-free media, followed by the addition of 1.5 μL of a lipid-polymer based mixture. The negatively charged DNA will interact with the positively charged lipids to form a complex that has an overall positive charge. When this complex is applied to cells, the complex is able to interact with the negatively charged cell membrane. This interaction allows for the eventual delivery of the DNA into the cell, where the cell machinery can transcribe the DNA into RNA and translate that RNA into protein.

The Tal-FokI proteins contain a nuclear localization signal that directs the proteins to the nucleus, where the target HIV DNA is found. If the Tal-FokI protein pair is able to bind and cleave the integrated target HIV-1 DNA, the cellular machinery will inherently attempt to “fix” the cleavage break in the target HIV-1 proviral DNA, but in a way that is easily detectable using DNA sequencing (i.e. it makes mistakes such as insertions or deletions of DNA sequence). To that end, we placed both 5′Tal-FokI and 3′Tal-FokI DNA constructs into U1/HIV-1 cells and then allowed 48 hours for protein expression. At the end of 48 hours, the U1/HIV-1 cells were collected by centrifugation at 1000 rpm for 3 minutes.

To begin harvesting the genomic DNA, the cells were first resuspended in 200 μL of 1×PBS (137 mM sodium chloride, 2.7 mM potassium chloride, 10 mM sodium phosphate dibasic, 1.8 mM potassium phosphate monobasic). To denature proteins and degrade RNA, 20 μL of Proteinase K (20 mg/mL) and 20 μL of RNase A (20 mg/mL) were added, followed by a brief vortexing (2 seconds) of the sample and incubation at 25° C. for 2 minutes. Upon completion of the incubation, 200 μL of lysis/binding buffer was added followed by a 10 minute incubation at 55° C. This step degraded proteins and broke open the cells. Following the 10 minute incubation, 200 μL of 95% ethanol was added to the sample, followed by vortexing for 5 seconds. At this point the sample contains the genomic DNA, denatured proteins, degraded RNA, chaotropic salts (guanidine hydrochloride), and ethanol. This mixture was applied to a silica membrane column to allow the DNA to bind to the membrane. Once the DNA was bound, the membrane was washed with buffers containing Tris-HCl and ethanol to remove impurities. Following washing the column, the DNA was eluted from the column with 50 μL elution buffer (10 mM Tris-HCl, pH 9.0, 0.1 mM EDTA). Once the genomic DNA was purified, polymerase chain reactions (pcrs) were performed to amplify (make many copies) the targeted region of the HIV-1 proviral DNA. The per reactions contained the following:

13 μL genomic DNA,

1 μL U3BamHI75For primer (10 μM),

1 μL GagSalI804Rev primer (10 μM),

15 μL pcr mix (Taq DNA polymerase,

KCl, MgCl, dNTPs, and (NH₄)₂SO₄).

The per reactions were run in a thermocycler with the following program:

1. 95° C. for 15 minutes (activate enzyme) (e.g., between 70-105° C. for at least 30 minutes at lower temperature to 10 minutes at elevated temperatures)
2. 94° C. for 45 seconds (denature DNA to make it accessible to primers) (e.g., between 70-100° C. for at least 60 seconds at lower temperature to about 30 seconds at elevated temperatures)
3. 60° C. for 45 seconds (anneal primers to DNA template) (e.g., between 45-80° C. for at least 60 minutes at lower temperature to about 40 seconds at elevated temperatures)
4. 72° C. for 1 minute (allow time for the DNA polymerase to extend the synthesized DNA product to its full size of 730 nucleotides) (e.g., between 55-85° C. for at least 2 minutes at lower temperature to about 50 seconds at elevated temperatures)
5. Go to 2, repeat over 10 times (e.g., over 20 times, over 30 times, typically we use 35 times (to amplify product)

### 6. Hold at 4° C. (e.g., 1-10° C.)

These per reactions were then run on a 2% low melting agarose DNA gel at 150 volts for 1.5 hours. The low melting agarose was used to allow for gel purification of the DNA.

To gel purify the desired DNA bands (730 nt size), a hand held UV light was used to visualize the DNA so that the bands could be excised from the gel using a clean razor blade. The bands were weighed and then 3 volumes of buffer containing chaotrophic salts and ethanol was added to the bands. The bands were dissolved in this solution by incubating the tube at 50° C. for 10 minutes. The tubes were cooled to room temperature for 5 minutes. A silica membrane column was pretreated with buffer to prepare it for binding DNA. After pretreatment, the sample was added to column to bind the DNA. The column was then washed twice with a buffer containing ethanol and a low amount of chaotrophic salt. These washes remove impurities from the column. The column was then air dried to 5 minutes to remove residual ethanol. To elute, 50 μL of elution buffer (10 mM Tris-HCl, pH 8.5) was added to the column. To be able to make thousands of copies of this pool of DNA to sequence, these DNA “inserts” need to be digested with restriction enzymes to create “sticky ends.” These sticky ends will allow the insert to be ligated into a DNA plasmid vector with corresponding sticky ends. To that end, the eluted DNA is restriction digested with BamHI and SalI (<5% of digest volume) in a 10× restriction digest buffer (100 mM sodium chloride, 50 mM Tris-HCl, 10 mM magnesium chloride, 1 mM dithiothreitol pH 7.9 at 25° C.) with 10× bovine serum albumin for 1 hour at 37° C. At the end of the incubation time, the digested sample was phenol/chloroform extracted twice to remove the enzymes and then precipitated to concentrate the DNA. The DNA was resuspended in 10 μL H20. Now the copies of the targeted region were can be individually inserted (ligated) into the prepared vector and transformed into bacteria.

The ligation reaction was performed at room temperature for 30 minutes. It consisted of 3 μL insert DNA, 1 μL prepared vector, 1 μL water, 5 μL 2× ligase buffer, and 1 μL ligase.

Once ligation is complete, the vector containing the insert (i.e., the plasmid) is “transformed” or taken up by commercially available specialized E. coli that have been chemically engineered to take up “foreign” DNA. The ligation reaction (10 μL) was added to 90 μL of chemically “competent” E. coli cells and incubated on ice for 30 minutes to allow the plasmid to stick to the bacterial membrane. This mix was then heat shocked at 42° C. for 30 seconds to allow the plasmid to enter the bacteria. The mix was then incubated on ice for 10 minutes followed by a 1 hour shaking incubation with 250 μL of luria broth. Following the one hour incubation, 250 μL of the mix was spread onto an ampicillin plate and the plate was incubated at 37° C. for 18 hours. This allowed for selection of bacteria that only contain the plasmid because the plasmid contains a gene that allows the bacteria to be resistant to the antibiotic ampicillin.

Once in the bacteria, many copies of the desired DNA was made. The bacteria was inoculated into a 2 mL culture of luria broth with ampicillin (100 μg/mL) and then allowed to grow for 18 hours at 37° C. in a shaker. The cells were then centrifuged at 13,200 rpm for 3 minutes to pellet the bacteria. The DNA was then purified from the bacteria.

To begin purification, the bacterial cell pellet was resuspended in 250 μL of resuspension buffer (50 mM Tris-Cl, pH 8.0, 10 mM EDTA, 100 μg/mL RNase A). Resuspension was followed by addition of 250 μL of lysis buffer (200 mM NaOH, 1% SDS). Lysis was followed by addition of 350 μL of neutralization buffer (3.0M potassium acetate, pH 5.5). At this point the cellular RNA has been degraded and the cellular proteins have been denatured. The sample was centrifuged to pellet the majority of cellular debris. The supernatant from this centrifugation was applied to a silica membrane column to bind the DNA. The column was washed with buffers containing low levels of chaotrophic salts and ethanol to remove contaminants. The DNA was eluted from the column with 50 μl elution buffer (10 mM Tris-HCl, pH 8.5).

The DNA samples were then sent for DNA sequencing with a sequencing primer designed to bind>100 nt upstream of the target site so that any indication of cleavage by the Tal-FokI proteins (insertions or deletions of DNA in the target site) could be detected. The DNA sequence files obtained were then aligned using a sequence alignment tool. The DNA sample sequences were compared to the template sequence of HIVNY5 (M38431). As seen below, in the DNA sequence alignment, the 5′Tal-FokI DNA binding site is TCTCTGGTTAGACC in line 434 while the 3′Tal-FokI DNA binding site is highlighted TAGCTAGGGAACCCACTGCTTA in line 494, the first occurrence of AGATCT in line 494. The target cleavage area is bolded in black. The asterisk found below the HIV1NY5 indicates that all of the DNA sequences (3A1-3A10) are identical (have the same nucleotide) at that position with regard to the reference sequence (HIV1NY5). The only exception of a single DNA base change (A to G) is in sample 3A6, the fourth “G” found outside of the target region in line 416. This is not indicative of successful cleavage by the Tal-FokI proteins, followed by DNA repair by the cellular machinery. This result supports our hypothesis that the control Tal-FokI protein pair would not be able to bind/cleave the target HIV-1 DNA region at a detectable level.

| | | |
|---|---|---|
| CS730-3A4_pGEX5 | 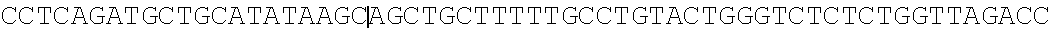 | 434 |
| CS730-3A8_pGEX5 | CCTCAGATGCTGCATATAAGCAGCTGCTTTTTGCCTGTACTGGGTCTCTCTGGTTAGACC | 430 |
| CS730-3A2_pGEX5 | CCTCAGATGCTGCATATAAGCAGCTGCTTTTTGCCTGTACTGGGTCTCTCTGGTTAGACC | 427 |
| CS730-3A10_pGEX5 | CCTCAGATGCTGCATATAAGCAGCTGCTTTTTGCCTGTACTGGGTCTCTCTGGTTAGACC | 423 |
| CS730-3A3_pGEX5 | CCTCAGATGCTGCATATAAGCAGCTGCTTTTTGCCTGTACTGGGTCTCTCTGGTTAGACC | 422 |
| CS730-3A6_pGEX5 | CCTCAGATGCTGCATATAGGCAGCTGCTTTTTGCCTGTACTGGGTCTCTCTGGTTAGACC | 416 |
| CS730-3A5_pGEX5 | CCTCAGATGCTGCATATAAGCAGCTGCTTTTTGCCTGTACTGGGTCTCTCTGGTTAGACC | 415 |
| CS730-3A7_pGEX5 | CCTCAGATGCTGCATATAAGCAGCTGCTTTTTGCCTGTACTGGGTCTCTCTGGTTAGACC | 415 |
| CS730-3A1_pGEX5 | CCTCAGATGCTGCATATAAGCAGCTGCTTTTTGCCTGTACTGGGTCTCTCTGGTTAGACC | 412 |
| CS730-3A9_pGEX5 | CCTCAGATGCTGCATATAAGCAGCTGCTTTTTGCCTGTACTGGGTCTCTCTGGTTAGACC | 408 |
| HIV1NY5 | CCTCAGATGCTGCATATAAGCAGCTGCTTTTTGCCTGTACTGGGTCTCTCTGGTTAGACC | 472 |
| | ****************** ***************************************** | |
| CS730-3A4_pGEX5 | 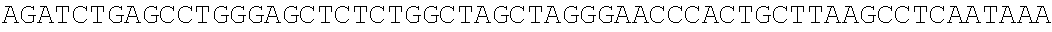 | 494 |
| CS730-3A8_pGEX5 | AGATCTGAGCCTGGGAGCTCTCTGGCTAGCTAGGGAACCCACTGCTTAAGCCTCAATAAA | 490 |
| CS730-3A2_pGEX5 | AGATCTGAGCCTGGGAGCTCTCTGGCTAGCTAGGGAACCCACTGCTTAAGCCTCAATAAA | 487 |
| CS730-3A10_pGEX5 | AGATCTGAGCCTGGGAGCTCTCTGGCTAGCTAGGGAACCCACTGCTTAAGCCTCAATAAA | 483 |
| CS730-3A3_pGEX5 | AGATCTGAGCCTGGGAGCTCTCTGGCTAGCTAGGGAACCCACTGCTTAAGCCTCAATAAA | 482 |
| CS730-3A6_pGEX5 | AGATCTGAGCCTGGGAGCTCTCTGGCTAGCTAGGGAACCCACTGCTTAAGCCTCAATAAA | 476 |
| CS730-3A5_pGEX5 | AGATCTGAGCCTGGGAGCTCTCTGGCTAGCTAGGGAACCCACTGCTTAAGCCTCAATAAA | 475 |
| CS730-3A7_pGEX5 | AGATCTGAGCCTGGGAGCTCTCTGGCTAGCTAGGGAACCCACTGCTTAAGCCTCAATAAA | 475 |
| CS730-3A1_pGEX5 | AGATCTGAGCCTGGGAGCTCTCTGGCTAGCTAGGGAACCCACTGCTTAAGCCTCAATAAA | 472 |
| CS730-3A9_pGEX5 | AGATCTGAGCCTGGGAGCTCTCTGGCTAGCTAGGGAACCCACTGCTTAAGCCTCAATAAA | 468 |
| HIV1NY5 | AGATCTGAGCCTGGGAGCTCTCTGGCTAGCTAGGGAACCCACTGCTTAAGCCTCAATAAA | 532 |
| | ************************************************************ | |

The following References provide background information for the technology and the examples are incorporated herein by reference. Chen S., Oikonomou G., Chiu C N, Niles B J, Liu J, Antoshechkin I, Prober D A. 2013. A large-scale in vivo analysis reveals that TALENs are significantly more mutagenic than ZFNs generated using context-dependent assembly. Nucleic Acids Research 1; 41(4): 2769-78. Ishida T, Hamano A, Koiwa T, Watanabe T. 2006. 5′ long terminal repeat (LTR)-selective methylation of latently infected HIV-1 provirus that is demethylated by reactivation signals. Retrovirology 12; 3:69. Emiliani S, Fischle W, Ott M, Van Lint C, Amella C A, Verdin E. 1998. Mutations in the tat gene are responsible for human immunodeficiency virus type 1 postintegration latency in the U1 cell line. Journal of Virology February; 72(2): 1666-70.

It is to be noted that as required in the presentation of the example, exact numbers, temperatures, concentrations and materials were specifically described to allow for authentication of the performed work. The specificity and exactness of these descriptions are not, however, intended to be absolute limitations on the practice of the present technologies, but are specific examples used to evidence the truly generic nature of the present technology. In some instances, additional ranges and estimates were provided. The absence of these voluntarily provided ranges is not an indication of a required specificity or exactness in the values provided. One skilled in the art appreciates that variations may be readily used in examples and practices based upon the generic teachings enabled in the present specification and descriptions.

It is to be further noted that as the genome surgery as described herein may be performed on a cell, and not necessarily on a cell within a patient as therapy, the generic concept of the present technology does not necessitate a medical treatment performed on a patient.

The present technology also includes a chemical tool for genome surgery comprising P2E2 constructs of, in order, a cell penetration component, a DNA binding component and a restriction endonuclease. Among the combinations of the restriction enzyme (endonuclease) and the target DNA sequences that can be cut are shown in the Table showing the sequence cuts (in alphabetical order) and corresponding enzyme names.

The chemical tool may include a restriction endonuclease is selected for targeting DNA in a HIV genome sequence embedded in a human genome and is linked to a restriction endonuclease effective for cutting sequences within the HIV genome sequence embedded in a human that repeats itself in parallel or antiparallel order such that the chemical tool is capable of cutting the HIV genome sequence embedded in the human genome a two distinct locations and thereby cut out a portion of the HIV genome sequence rather than make only a single cut in the HIV genome sequence.

The chemical tool may be constructed wherein the targeted DNA binding site in the HIV sequence is selected from the group consisting of TCTCTGGTTAGACC, TAGCTAGGGAACCCACTGCTTA or a smaller sequence of at least 6 nucleic acids within TCTCTGGTTAGACC or TAGCTAGGGAACCCACTGCTA.

The chemical tool may be specific to the restriction endonuclease being capable of cutting the HIV genome sequence within a sequence of GAGCCTGGAGCTCTCTGGC.

The present technology also includes a chemical tool for genome surgery comprising P2E2 constructs of, in any order, a cell penetration component, a DNA binding component and a restriction endonuclease. Among the combinations of the restriction enzyme (endonuclease) and the target DNA sequences that can be cut are shown in the Table showing the sequence cuts (in alphabetical order) and corresponding enzyme names.

The chemical tool may include a restriction endonuclease is selected for targeting DNA in a HIV genome sequence embedded in a human genome and is linked to a restriction endonuclease effective for cutting sequences within the HIV genome sequence embedded in a human that repeats itself in parallel or antiparallel order such that the chemical tool is capable of cutting the HIV genome sequence embedded in the human genome a two distinct locations and thereby cut out a portion of the HIV genome sequence rather than make only a single cut in the HIV genome sequence.

The chemical tool may be constructed wherein the targeted DNA binding site in the HIV sequence is selected from the group consisting of TCTCTGGTTAGACC, TAGCTAGGGAACCCACTGCTTA or a smaller sequence of at least 6 nucleic acids within TCTCTGGTTAGACC or TAGCTAGGGAACCCACTGCTA.

The chemical tool may be specific to the restriction endonuclease being capable of cutting the HIV genome sequence within a sequence of GAGCCTGGAGCTCTCTGGC.

The chemical tool may have an order of the components in the tool are selected from the group consisting of a) a cell penetration component, a DNA binding component and a restriction endonuclease and b) a cell penetration component, a restriction endonuclease, and a DNA binding component. The chemical tool may have a target sequence within the genome of Sac 1 or Fok1, for example.

The chemical tool may have an order of the components in the tool are selected from the group consisting of a) a cell penetration component, a DNA binding component and a restriction endonuclease and b) a cell penetration component, a restriction endonuclease, and a DNA binding component. The chemical tool may have a target sequence within the genome of Sac 1 or Fok1, for example.

Once the P2E2 proteins mediate cleavage of the HIV DNA, there are two methods of inactivation: (1) the P2E2 proteins cleave the HIV genome in two distinct sites (double strand cleavage at each site) and then the two ends of the genome are ligated to each other by cellular mechanisms such as non-homologouse end joining (NHEJ). (2) the P2E2 proteins cleave the HIV genome at one or more sites and cellular repair mechanisms such as NHEJ relegate the cleaved site. However, during this process mistakes are made where short segments up to 40 nucleotides are either inserted or deleted. This inactivates the virus.

U1 cells harbor a latent copy of HIV1 in their genome and can be grown in cell culture. These cells can be treated with Tumor Necrosis Factor alpha (TNFα) to wake up the latent virus. To test if P2E2 constructs can mutate the latent genomic copy of HIV1 in U cells, cultures were treated with 1 ng/ml TNFα for 1 day, transfected with 1 μg of each P2E2 construct, and then harvested after 2 days. Genomic DNA was recovered from cells using the PureLink™ genomic DNA minikit (InVitrogen). A 730 base pair region encompassing the 5′ LTR of the HIV genome containing the site targeted by the P2E2 constructs was amplified by PCR and purified. The purified DNA was digested with the Sac 1 endonuclease to determine if this site had been destroyed in the HIV genome. FIG. 14 (upper panel) shows nearly complete cleavage of the HIV genomic DNA fragment in cells not treated with P2E2 constructs (control); however, nearly half of the HIV genomic DNA fragment was not cleaved in the PCR product prepared from U1 cells treated with the P2E2 constructs. In a separate experiment with hEK-293 cells, Western blot analysis of cells transfected with P2E2 constructs shows that the proteins are expressed in cells (lower panel). Importantly, this result indicates that the P2E2 constructs can cleave HIV genomic DNA in cells containing a latent genomic copy of HIV1. This experiment serves as a proof-of-principle of an approach to cure or reduce the load of HIV viral latency and is most like applicable to other latent viruses.

1. A method for performing genome surgery comprising:
a) providing one or more recombinant P2E2 constructs comprising a cell penetration component, a DNA binding component and a endonuclease;
b) penetrating a cell with the recombinant P2E2 protein construct;
c) forming a protein product in the cell by the processes of transcription and translation or by direct introduction of the P2E2 protein construct to the cell;
d) attaching the protein product of the P2E2 construct to one or more targeted genomic sequences within the cell; and
e) the endonuclease of the P2E2 construct cutting both strands of the genome at target locations.

2. The method of claim 1 wherein the cell is penetrated by the recombinant P2E2 constructs comprising a purified P2E2 protein through a process selected from the group consisting of i) introduction to cells with a viral vector encoding the P2E2 construct, ii) transfection of cells with the P2E2 construct using a transfection strategies and iii) application of a recombinant protein purified from E. coli, yeast, insect, or mammalian cells transfected, transformed, or infected with a vector encoding the P2E2 construct.

3. The method of claim 1 wherein the cell is penetrated by one or more P2E2 proteins through a cell penetration process in which the recombinant protein is delivered by direct application or is bound to a carrier molecule and delivered.

4. The method of claim 1 wherein cutting of both strands is at site(s) within the genome that are within genome segments that include targeted regions that contain some base pair mismatches.

5. A method for performing genome surgery comprising:
a) providing a P2E2 protein comprising, a cell penetration component, a DNA binding component and a endonuclease;
b) penetrating a cell with the recombinant P2E2 constructs or proteins;
c) attaching individual P2E2 recombinant protein to respective target sites on two strands of the genome within the cell, the attaching of the two individual recombinant proteins positioning the endonuclease of each recombinant protein over a pair of sequences opposed to each other across a gap between the two strands of the genome; and
d) the endonucleases of each P2E2 recombinant protein cutting both strands of the genome at each of their respective target sites.

6. The method of claim 5 wherein the endonuclease of the P2E2 recombinant protein cuts both strands of the genome at identical respective target sites.

7. The method of claim 1 wherein penetrating of the cell is performed by a method selected from the group consisting of a) introduction to cells with any viral vector encoding the P2E2 recombinant protein, b) transfection of cells with the P2E2 recombinant proteins using a transfection strategy, c) microinjection of a P2Ew encoding plasmid, mRNA, protein, or protein conjugate, and d) direct application of a recombinant protein encoded by the P2E2 constructs that has been purified from E. coli, Yeast, Insect cells, or other protein expression systems.

8. The method of claim 3 wherein penetrating of the cell is performed by a method selected from the group consisting of a) introduction to cells with a viral vector encoding the P2E2 recombinant protein, b) transfection of cells with the P2E2 recombinant proteins using a transfection strategy and c) application of a recombinant protein encoded by the P2E2 constructs that have been purified from E. coli, yeast, insect cells or other protein expression systems.

9. The method of claim 6 wherein penetrating of the cell is performed by a method selected from the group consisting of a) introduction to cells with a viral vector encoding the P2E2 recombinant protein, b) transfection of cells with the P2E2 recombinant proteins using a transfection strategy or biolistic particle gun and c) application of a recombinant protein encoded by the P2E2 recombinant protein that has been purified from E. coli, yeast, insect cells or other protein expression systems.

10. A method for performing genome surgery on an integrated viral genome comprising:
a) identifying an integrated viral genome within a host genome;
b) identifying a target region of nucleic acid sequences within the integrated viral genome;
c) providing a P2E2 recombinant protein comprising a cell penetration component, a DNA binding component and a endonuclease;
d) penetrating a cell with the recombinant P2E2 recombinant protein;
e) attaching the P2E2 recombinant protein to a genome consisting of a viral integrated genome within a host genome within the cell; and
f) the endonuclease of the P2E2 recombinant protein overlaying a section of the integrated viral genome; and
g) cutting a strand of the integrated viral genome within the cell.

11. The method of claim 10 wherein the endonuclease of the P2E2 recombinant protein cuts both strands of the genome at identical respective target regions.

12. The method of claim 11 wherein ends of each cut strand of the integrated viral genome reattach within the cell with attendant genetic rearrangement forming an altered nucleic acid sequence as compared to the nucleic acid sequence of the integrated viral genome before cutting of the strand.

13. (canceled)

14. The method of claim 12 wherein the integrated viral genome has two ends through which the integrated viral genome is covalently inserted within the host genome, and a pair of P2E2 recombinant proteins attach at each of the two ends so that the endonuclease of each of the recombinant proteins overlay a section of the integrated viral genome, and two strands between each of the two ends of the integrated viral genome are cut. forming a segment of the previously integrated viral genome that is excised from the host genome.

15. (canceled)

16. The method of claim 5 wherein two distinct and different pairs of P2E2 recombinant proteins are simultaneously or consecutively used in steps a), b) and c) and in step d), a total of 4 DNA strand cuts are made, with two cuts each by each pair of P2E2 constructs.

17. The method of claim 5 wherein the genome segment comprises an HIV genome segment.

18. The method of claim 17 wherein only single type of P2E2 recombinant protein is used to make four cuts on identical genome sequences in the HIV genome segment.

19. The method of claim 17 wherein only at least two pairs of P2E2 recombinant proteins are used to make four cuts on two different sites on the HIV genome segment.

20. The method of claim 1 wherein the order of the components in the construct are selected from the group consisting of a) a cell penetration component, a DNA binding component and a restriction endonuclease and b) a cell penetration component, a restriction endonuclease, and a DNA binding component.

21. A chemical tool for genome surgery comprising P2E2 constructs of a cell penetration component, a DNA binding component and a restriction endonuclease.

22-34. (canceled)
